(19) United States
(12) Patent Application Publication
(10) Pub. No.: US 2014/0314667 A1
(43) Pub. Date: Oct. 23, 2014

(54) METHODS OF TREATING EPIDERMAL GROWTH FACTOR DELETION MUTANT VIII RELATED DISORDERS

(71) Applicant: AMGEN INC., Thousand Oaks, CA (US)

(72) Inventors: John Stephen Hill, Solvang, CA (US); Kevin J. Hamblett, Seattle, WA (US)

(73) Assignee: AMGEN INC., Thousand Oaks, CA (US)

(21) Appl. No.: 14/358,641

(22) PCT Filed: Nov. 16, 2012
(86) PCT No.: PCT/US2012/065707

§ 371 Date: May 15, 2014

Related U.S. Application Data
(60) Provisional application No. 61/727,029, filed on Nov. 15, 2012.
(60) Provisional application No. 61/560,731, filed on Nov. 16, 2011.

(51) Int. Cl. A61K 47/48; A61K 45/06

(52) U.S. Cl. CPC A61K 47/48561; A61K 45/06
USPC 424/1.49; 424/178.1; 424/183.1; 424/181.1

(57) ABSTRACT

The present invention relates to methods of treating treating epidermal growth factor deletion mutant vIII (EGFRvIII) related disorders, such as glioblastoma or anaplastic astrocyte tumors, using antigen binding proteins, including antibodies against EGFRvIII conjugated to a drug. Diagnostic and therapeutic formulations of such antibodies and drug conjugates thereof are also provided.

This application claims the benefit of U.S. Provisional Application No. 61/727,029 filed Nov. 15, 2012, and U.S. Provisional Application No. 61/560,731 filed Nov. 16, 2011 which are incorporated by reference herein.

### REFERENCE TO THE SEQUENCE LISTING

The present application is being filed along with a Sequence Listing in electronic format. The Sequence Listing is provided as a file entitled A-1680-US-PSP_SEQ.txt created Nov. 16, 2011 which is 89 KB in size. The information in the electronic format of the Sequence Listing is incorporated herein by reference in its entirety.

### FIELD OF THE INVENTION

The present invention relates to methods of treating treating epidermal growth factor deletion mutant vIII (EGFRvIII) related disorders, such as glioblastoma or anaplastic astrocyte tumors, using antigen binding proteins, including antibodies against EGFRvIII conjugated to a drug. Diagnostic and therapeutic formulations of such antibodies and drug conjugates thereof are also provided.

### BACKGROUND OF THE INVENTION

Tumor specific molecules to aid in better diagnosis and treatment of human and animal cancer have been sought since the last century. Hard evidence of tumor-specific substances, based on molecular structural data, has been difficult to provide in most types of human cancer except those based on virally-induced cancer and involving molecular structures specified by the virus genome. There have been extremely few examples of tumor-specific molecules based on novel molecular structures. In the case of malignant human gliomas and other tumors potentially associated with amplification or changes in the epidermal growth factor receptor molecule, such as carcinoma of the breast and other human carcinomas, there have been no unequivocal demonstrations of structurally altered molecules with unique sequences.

The epidermal growth factor receptor (EGFR) is the 170 kilodalton membrane glycoprotein product of the proto-oncogene c-erb B. The sequence of the EGFR gene is known (Ullrich et al. (1984). Human Epidermal Growth Factor Receptor cDNA Sequence and Aberrant Expression of the Amplified Gene in A431 Epidermoid Carcinoma Cells. Nature 309:418-425). The EGFR gene is the cellular homolog of the erb B oncogene originally identified in avian erythroblastosis viruses (Downward et al. (1984). Close Similarity of Epidermal Growth Factor Receptor and v-erb B Oncogene Protein Sequence. Nature 307:521-527, Ullrich, et al. (1984)). Activation of this oncogene by gene amplification has been observed in a variety of human tumors (Haley et al. (1987A). The Epidermal Growth Factor Receptor Gene in: Oncogenes, Genes, and Growth Factors Edited by: Guroff, G. 12th Edition. Chapter 2. pp. 40-76. Wiley, N.Y.), and in particular, those of glial origin (Libermann et al. (1985). Amplification, Enhanced Expression and Possible Rearrangement of EGF Receptor Gene in Primary Human Brain Tumours of Glial Origin. Nature 313:144-147; Wong et al. (1987). Increased Expression of the Epidermal Growth Factor Receptor Gene in Malignant Gliomas is Invariably Associated with Gene Amplification. Proc. Natl. Acad. Sci. USA 84:6899-6903; Yamazaki et al. (1988). Amplification of the Structurally and Functionally Altered Epidermal Growth Factor Receptor Gene (c-erbB) in Human Brain Tumors. Molecular and Cellular Biology 8:1816-1820; Malden et al., (1988). Selective Amplification of the Cytoplasmic Domain of the Epidermal Growth Factor Receptor Gene in Glioblastoma Multiforme. Cancer Research 4:2711-2714).

EGF-r has been demonstrated to be overexpressed on many types of human solid tumors. Mendelsohn Cancer Cells 7:359 (1989), Mendelsohn Cancer Biology 1:339-344 (1990), Modjtahedi and Dean Int'l J. Oncology 4:277-296 (1994). For example, EGFR overexpression has been observed in certain lung, breast, colon, gastric, brain, bladder, head and neck, ovarian, kidney and prostate carcinomas. Modjtahedi and Dean Int'l J. Oncology 4:277-296 (1994). Both epidermal growth factor (EGF) and transforming growth factor-alpha (TGF-alpha.) have been demonstrated to bind to EGF-r and to lead to cellular proliferation and tumor growth.

One major difference between v-erb B oncogenes and the normal EGFR gene is that the viral oncogenes are amino-truncated versions of the normal receptor; they lack most of the extracytoplasmic domain but retain the transmembrane and tyrosine kinase domains (Fung et al., (1984) Activation of the Cellular Oncogene c-erb B by LTR Insertion: Molecular Basis for Induction of Erythroblastosis by Avian Leukosis Virus. Cell 33:357-368; Yamamoto et al., (1983). A New Avain Erythroblastosis Virus, AEV-H Carries erbB Gene Responsible for the Induction of Both Erythroblastosis and Sarcoma. Cell 34:225-232, Nilsen et al., (1985). c-erbB Activation in ALV-Induced Erythroblastosis: Novel RNA Processing and Promoter Insertion Results in Expression of an Amino-Truncated EGF Receptor. Cell 41:719-726; Gammett et al., (1986). Differences in Sequences Encoding the Carboxy-Terminal Domain of the Epidermal Growth Factor Receptor Correlate with Differences in the Disease Potential of Viral erbB Genes. Proc. Natl. Acad. Sci. USA 83:6053-6057). This results in a protein that is unable to bind epidermal growth factor (EGF) but can still phosphorylate other substrates (Gilmore et al., (1985). Protein Phosphorlytion at Tyrosine is Induced by the v-erb B Gene Product in Vivo and In Vitro. Cell 40:609-618; Kris et al., (1985). Antibodies Against a Synthetic Peptide as a Probe for the Kinase Activity of the Avian EGF Receptor and v-erB Protein. Cell 40:619-625), and has led to speculation that the v-erb B proteins are oncogenic because the kinase domain is unregulated and constitutively active (Downward et al., 1984).

A variety of genetic alterations can occur in viral erb B oncogenes, e.g. amino acid substitutions and deletions in the carboxy terminus of the gene. Available evidence, however, argues that the amino truncation is critical to carcinogenesis. Amino truncations are a feature of all v-erb B oncogenes, including those that arise by promoter insertion or retroviral transduction (Nilsen et al., (1985). c-erbB Activation in ALV-Induced Erythroblastosis: Novel RNA Processing and Promoter Insertion Results in Expression of an Amino-Truncated EGF Receptor. Cell 41:719-726; Gammett et al., (1986). Differences in Sequences Encoding the Carboxy-Terminal Domain of the Epidermal Growth Factor Receptor Correlate with Differences in the Disease Potential of Viral erbB Genes. Proc. Natl. Acad. Sci. USA 83:6053-6057).

In contrast, carboxy-terminal deletions appear to be associated only with tumors that arise through retroviral transduction and seem to determine host range and tumor type specificity (Gammett et al., 1986; Raines et al., (1985). c-erbB Activation in Avian Leukosis Virus-Induced Erythroblastosis: Clustered Integration Sites and the Arrangement of Provirus in the c-erbB Alleles. Proc. Natl. Acad. Sci. USA 82:2287-2291). Transfection experiments with amino-truncated avian c-erb B genes or chimeric viral oncogene-human EGF receptors demonstrates that this deletion is sufficient alone to create a transforming protein (Pellet′ et al., (1988). Proviral-Activated c-erbB is Leukemogenic but not Sarcomagenic: Characterization of a Replication—Competent Retrovirus Containing the Activated c-erbB. Journal of Virology 62: 1840-1844; Wells et al., (1988). Genetic Determinants of Neoplastic Transformation by the Retroviral Oncogene v-erbB. Proc. Natl. Acad. Sci. USA 85:7597-7601).

Amplification of the EGFR gene occurs in approximately 40% of malignant human gliomas (Libermann et al., (1985) Amplification, Enhanced Expression and Possible Rearrangement of EGF Receptor Gene in Primary Human Brain Tumours of Glial Origin. Nature 313:144-147; Wong et al., (1987). Increased Expression of the Epidermal Growth Factor Receptor Gene in Malignant Gliomas is Invariably Associated with Gene Amplification. Proc. Natl. Acad. Sci. USA 84:6899-6903), Rearrangement of the receptor gene is evident in many of the tumors with gene amplification. The structural alterations seem to preferentially affect the amino terminal half of the gene (Yamazaki et al., (1985). Amplification, Enhanced Expression and Possible Rearrangement of EGF Receptor Gene in Primary Human Brain Tumours of Glial Origin. Nature 313:144-147; Malden et al., (1988). Selective Amplification of the Cytoplasmic Domain of the Epidermal Growth Factor Receptor Gene in Glioblastoma Multiforme. Cancer Research 4:2711-2714), but the nature of the rearrangements had not at that time been precisely characterized in any tumor.

Size variant EGFR genes and amplification have been reported in several human cancers. (Humphrey et al., (1988). Amplification and Expression of the Epidermal Growth Factor Receptor Gene in Human Glioma Xenografts. Cancer Research 48:2231-2238; Bigner et al., (1988) J. Neuropathol. Exp. Neurol., 47:191-205; Wong et al., (1987). Increased Expression of the Epidermal Growth Factor Receptor Gene in Malignant Gliomas is Invariably Associated with Gene Amplification. Proc. Natl. Acad. Sci. USA 84:6899-6903; and Humphrey et al. Amplification and expression of the epidermal growth factor receptor gene in human glioma xenografts. Cancer Res. 48(8):2231-8 (1988)) There had been no determination, however, of the molecular basis for the altered EGFR molecules in cells.

In 1989, work of Drs. Bigner and Vogelstein elucidated the sequence of a EGF receptor mutant that has become known as the type III mutant (also referred to as delta-EGFr or EGFRvIII). This work is described in U.S. Pat. Nos. 6,455,498, 6,127,126, 5,981,725, 5,814,317, 5,710,010, 5,401,828, and 5,212,290, the disclosures of which are hereby incorporated by reference.

EGFR variants are caused by gene rearrangement accompanied by EGFR gene amplification. There are eight major variants of EGFR that are known: (i) EGFRvI lacks a majority of the extracellular domain of EGFR, (ii) EGFRvII consists of an 83 aa in-frame deletion in the extracellular domain of EGFR, (iii) EGFRvIII consists of a 267 aa in-frame deletion in the extracellular domain of EGFR, (iv) EGFRvIV contains deletions in the cytoplasmic domain of EGFR, (v) EGFRvV contains deletions in cytoplasmic domain of EGFR, (vi) EGFR.TDM/2-7 contains a duplication of exons 2-7 in the extracellular domain of EGFR, (vii) EGFR.TDM/18-25 contains a duplication of exons 18-26 in the tyrosine kinase domain of EGFR, and (viii) EGFR.TDM/18-26 contains a duplication of exons 18-26 in the tyrosine kinase domain of EGFR (Kuan et al. EGF mutant receptor vIII as a molecular target in cancer therapy. Endocr Relat Cancer. 8(2):83-96 (2001)). In addition, there is a second, more rare, EGFRvIII mutant (EGFRvIII/Δ12-13) that possesses a second deletion that introduces a novel histidine residue at the junction of exons 11 and 14 (Kuan et al. EGF mutant receptor vIII as a molecular target in cancer therapy. Endocr Relat Cancer. 8(2):83-96 (2001)).

EGFRvIII is the most commonly occurring variant of the epidermal growth factor (EGF) receptor in human cancers (Kuan et al. EGF mutant receptor vIII as a molecular target in cancer therapy. Endocr Relat Cancer. 8(2):83-96 (2001)). During the process of gene amplification, a 267 amino acid deletion occurs in the extracellular domain creating a novel junction to which tumor specific monoclonal antibodies can be directed. This variant of the EGF receptor contributes to tumor progression through constitutive signaling in a ligand independent manner. EGFRvIII is not known to be expressed on any normal tissues (Wikstrand, C J. et al. Monoclonal antibodies against EGFRvIII are tumor specific and react with breast and lung carcinomas malignant gliomas. Cancer Research 55(14): 3140-3148 (1995); Olapade-Olaopa, E O. et al. Evidence for the differential expression of a variant EGF receptor protein in human prostate cancer. Br J Cancer. 82(1):186-94 (2000. The deletion of 267 amino acids with a Glycine substitution creates a unique junction that may be capable of antibody targeting. Further, in view of EGFRvIII's expression in certain tumors and its lack of expression in normal tissues, EGFRvIII may be an ideal target for drug targeting in tumor therapy. In particular, EGFRvIII would appear to be an ideal candidate for immunoconjugate therapy of tumors (e.g., an antibody conjugated to an antineoplastic agent or toxin). Another method of treatment of cancers which over-express EGFRvIII involved the use of a tumor-specific ribozyme targeted specifically to the variant receptor which did not cleave normal EGFR. The ribozyme was found to significantly inhibit breast cancer growth in athymic nude mice (Luo et al. Int. J. Cancer. 104(6):716-21 (2003)).

General antibodies for the entire EGFRvIII protein have been described. See International Patent Application No. WO 01/62931 and Kuan et al. EGF mutant receptor vIII as a molecular target in cancer therapy. Endocr Relat Cancer. 8(2):83-96 (2001), Kuan et al. EGFRvIII as a promising target for antibody-based brain tumor therapy. Brain Tumor Pathol. 17(2):71-78 (2000), Kuan et al. Increased binding affinity enhances targeting of glioma xenografts by EGFRvIII-specific scFv. International Journal of Cancer. 88(6):962-969 (2000), Landry et al. Antibody recognition of a conformational epitope in a peptide antigen: Fv-peptide complex of an antibody fragment specific for the mutant EGF receptor, EGFRvIII. Journal of Molecular Biology. 308(5):883-893 (2001), Reist et al. Astatine-211 labeling of internalizing anti-EGFRvIII monoclonal antibody using N-succinimidyl 5-[211At]astato-3-pyridinecarboxylate. Nuclear Medicine and Biology. 26(4):405-411 (1999), Reist et al. In vitro and in vivo behavior of radiolabeled chimeric anti-EGFRvIII monoclonal antibody: comparison with its murine parent. Nuclear Medicine and Biology. 24(7):639-647 (1997), Wikstrand et al. Generation of anti-idiotypic reagents in the EGFRvIII tumor-associated antigen system. Cancer Immunology, Immunotherapy. 50(12):639-652 (2002), Wikstrand et al. Monoclonal antibodies against EGFRvIII are tumor specific and react with breast and lung carcinomas malignant gliomas. Cancer Research. 55(14):3140-3148 (1995), Wikstrand et al. The class III variant of the epidermal growth factor receptor (EGFR_{V}III): characterization and utilization as an immunotherapeutic target. J. Neurovirol. 4(2):148-158 (1998), Wikstrand et al. The class III variant of the epidermal growth factor receptor (EGFRvIII): characterization and utilization as an immunotherapeutic target. J. Neurovirol. 4(2):148-158 (1998), Jungbluth et al. A monoclonal antibody recognizing human cancers with amplification/overexpression of the human epidermal growth factor receptor. Proc Natl Acad Sci USA. 100(2):639-44 (2003), Mamot et al. Epidermal Growth Factor Receptor (EGFR)-targeted Immunoliposomes Mediate Specific and Efficient Drug Delivery to EGFR- and EGFRvIII-overexpressing Tumor Cells. Cancer Research 63:3154-3161 (2003)). Each of these above-mentioned antibodies, however, possess or contain murine sequences in either the variable and/or constant regions. The presence of such murine derived proteins can lead to the rapid clearance of the antibodies or can lead to the generation of an immune response against the antibody in a patient. In addition, such antibodies are relatively low affinity, on the order of 2.2×10⁻⁸ through 1.5×10⁻⁹, even after affinity maturation. (Kuan et al. EGF mutant receptor vIII as a molecular target in cancer therapy. Endocr Relat Cancer. 8(2):83-96 (2001)).

In order to avoid the utilization of murine or rat derived antibodies, researchers have introduced human antibody function into rodents so that the rodents can produce fully human antibodies. See e.g., Mendez et al. Functional transplant of megabase human immunoglobulin loci recapitulates human antibody response in mice. Nat Genet. 15(2):146-56 (1997). This approach has been used in connection with the generation of successful antibodies directed against wild type EGFR. See e.g., Yang X et al. Development of ABX-EGF, a fully human anti-EGF receptor monoclonal antibody, for cancer therapy. Crit Rev Oncol Hemato 38(1):17-23 (2001); Yang X-D et al. Eradication of Established Tumors by a Fully Human Monoclonal Antibody to the Epidermal Growth Factor Receptor without Concomitant Chemotherapy. Cancer Research 59(6):1236-1243 (1999); and U.S. Pat. No. 6,235,883.

### SUMMARY OF THE INVENTION

In one embodiment, the invention comprises an isolated human monoclonal antibody, conjugated to a toxin, particularly DM1, that specifically binds to EGFRvIII and a peptide that comprises the sequence L E E K K G N Y V V T D H C (SEQ ID NO: 56) wherein the antibody is conjugated to a toxin, particularly DM1. In another embodiment, the invention comprises an isolated human monoclonal antibody conjugated to a toxin, particularly DM1, that specifically binds to an epitope contained within a sequence comprising L E E K K G N Y V V T D H C (SEQ ID NO: 56), wherein the residues required for binding, as determined by Alanine scanning in a SPOTs array, are selected from the group consisting of EEK, KKNYV, LEK, EKNY and EEKGN.

Further embodiments include an isolated human monoclonal antibody conjugated to a toxin, particularly DM1, that comprises a heavy chain variable region amino sequence that is encoded by a VH3-33 gene. The heavy chain variable region amino sequence can include an amino acid sequence that is encoded by a JH4b gene, or an amino acid sequence that is encoded by a D gene that is selected from the group consisting of D6-13 and D3-9.

Other embodiments include an isolated human monoclonal antibody conjugated to a toxin, particularly DM1, that comprises a light chain variable region amino sequence that is encoded by a A23(VK2) gene. The light chain variable region amino sequence can include an amino acid sequence that is encoded by a JK1 gene.

Other embodiments include an isolated antibody, or fragment thereof, that binds to EGFRvIII, is conjugated to a toxin, particularly DM1, and that comprises a heavy chain amino acid sequence selected from the group consisting of the heavy chain amino acid sequence of antibody 13.1.2, 131, 170, 150, 095, 250, 139, 211, 124, 318, 342 and 333 as identified in (SEQ ID NO: 138, 2, 4, 5, 7, 9, 10, 12, 13, 15, 16, and 17). The antibody can be a monoclonal antibody, a chimeric antibody, a humanized antibody or a human antibody.

The toxin can be associated with the antibody via a linker. The toxin can be associated with the antibody via a secondary antibody. Further embodiments include a hybridoma cell line producing the antibody, and a transformed cell comprising a gene encoding the antibody. The cell can be, for example, a Chinese hamster ovary cell.

Further embodiments include a method of inhibiting cell proliferation associated with the expression of EGFRvIII, comprising treating cells expressing EGFRvIII with an effective amount of the antibody or fragment. In one embodiment, the antibody, is conjugated to a toxin, particularly DM1, and comprises a heavy chain amino acid sequence selected from the group consisting of the heavy chain amino acid sequence of antibody 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17). The method can be performed in vivo, and performed on a mammal, such as a human, who suffers from a cancer involving epithelial cell proliferation, such as a lung, colon, gastric, renal, prostate, breast, head and neck or ovarian carcinoma or glioblastoma.

Further embodiments include a method of killing a targeted cell. This is achieved by contacting the targeted cell with an antibody associated with a toxin. The antibody binds to a peptide LEEKKGNY (SEQ ID NO: 133). In one embodiment, the antibody has a binding affinity greater than 1.3*10⁻⁹M to the peptide. In one embodiment the toxin is DM1. In one embodiment, the antibody toxin compound is 10 fold more toxic to targeted cells than to cells without the peptide. In one embodiment, the antibody comprises a heavy chain amino acid sequence selected from the group consisting of the heavy chain amino acid sequence of antibody 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17). In another embodiment, the antibody is associated with a toxin via a non-cleavable linker.

Further embodiments of the invention include an isolated antibody, conjugated to a toxin, particularly DM1, that binds to EGFRvIII and that comprises a heavy chain amino acid sequence comprising the following complementarity determining regions (CDRs):

(a) CDR1 consisting of a sequence selected from the group consisting of the amino acid sequences for the CDR1 region of antibodies 13.1.2, 131, 170, 150, 095, 250, 139, 211, 124, 318, 342 and 333 as identified in SEQ ID NO: 138, 2, 4, 5, 7, 9, 10, 12, 13, 15, 16, and 17;
(b) CDR2 consisting of a sequence selected from the group consisting of the amino acid sequences for the CDR2 region of antibodies 13.1.2, 131, 170, 150, 095, 250, 139, 211, 124, 318, 342 and 333 as identified in SEQ ID NO: 138, 2, 4, 5, 7, 9, 10, 12, 13, 15, 16, and 17; and
(c) CDR3 consisting of a sequence selected from the group consisting of the amino acid sequences for the CDR3 region of antibodies 13.1.2, 131, 170, 150, 095, 250, 139, 211, 124, 318, 342 and 333 as identified in SEQ ID NO: 138, 2, 4, 5, 7, 9, 10, 12, 13, 15, 16, and 17. In one embodiment, the conjugated antibody is a monoclonal antibody, a chimeric antibody, human, or a humanized antibody. In one embodiment, the conjugated antibody is associated with a pharmaceutically acceptable carrier, diluent, and/or therapeutic agent.

Also included is an isolated antibody, or fragment thereof, conjugated to a toxin, particularly DM1, that binds to EGFRvIII and that comprises a light chain amino acid sequence selected from the group consisting of the light chain amino acid sequence of antibody 13.1.2, 131, 170, 150, 123, 095, 139, 250, 211, 318, 342, and 333 as identified in SEQ ID NO: 140, 19, 20, 21, 29, 23, 25, 26, 28, 33, 31 and 32. The conjugated antibody can be a monoclonal antibody, a chimeric antibody, a humanized antibody, or a human antibody. It can be associated with a pharmaceutically acceptable carrier or diluents.

In one embodiment a hybridoma cell line or a transformed cell producing an antibody comprising a light chain amino acid sequence selected from the group consisting of the light chain amino acid sequence of antibody 13.1.2, 131, 170, 150, 123, 095, 139, 250, 211, 318, 342, and 333 as identified in SEQ ID NO: 140, 19, 20, 21, 29, 23, 25, 26, 28, 33, 31 and 32 is contemplated.

Yet another embodiment includes a method of inhibiting cell proliferation associated with the expression of EGFRvIII, comprising treating cells expressing EGFRvIII with an effective amount of the conjugate antibodies or fragments described above. The method can be performed in vivo and in a mammal, such as a human, who suffers from a cancer involving epithelial cell proliferation such as lung, colon, gastric, renal, prostate, breast, head and neck ovarian carcinoma or glioblastoma.

Yet another embodiment includes an isolated antibody conjugated to a toxin, particularly DM1, that binds to EGFRvIII and that comprises a light chain amino acid sequence comprising the following complementarity determining regions (CDRs):

(a) CDR1 consisting of a sequence selected from the group consisting of the amino acid sequences for the CDR1 region of antibodies 13.1.2, 131, 170, 150, 123, 095, 139, 250, 211, 318, 342, and 333 as identified in SEQ ID NO: 140, 19, 20, 21, 29, 23, 25, 26, 28, 33, 31 and 32;
(b) CDR2 consisting of a sequence selected from the group consisting of amino acid sequences for the CDR1 region of antibodies 13.1.2, 131, 170, 150, 123, 095, 139, 250, 211, 318, 342, and 333 as identified in SEQ ID NO: 140, 19, 20, 21, 29, 23, 25, 26, 28, 33, 31 and 32; and
(c) CDR3 consisting of a sequence selected from the group consisting of amino acid sequences for the CDR1 region of antibodies 13.1.2, 131, 170, 150, 123, 095, 139, 250, 211, 318, 342, and 333 as identified in SEQ ID NO: 140, 19, 20, 21, 29, 23, 25, 26, 28, 33, 31 and 32.

The conjugated antibody identified in the previous paragraph can further include a heavy chain amino acid sequence comprising the following complementarity determining regions (CDRs):

(a) CDR1 consisting of a sequence selected from the group consisting of the amino acid sequences for the CDR1 region of antibodies 13.1.2, 131, 170, 150, 095, 250, 139, 211, 124, 318, 342 and 333 as identified in SEQ ID NO: 138, 2, 4, 5, 7, 9, 10, 12, 13, 15, 16, and 17;
(b) CDR2 consisting of a sequence selected from the group consisting of the amino acid sequences for the CDR2 region of antibodies 13.1.2, 131, 170, 150, 095, 250, 139, 211, 124, 318, 342 and 333 as identified in SEQ ID NO: 138, 2, 4, 5, 7, 9, 10, 12, 13, 15, 16, and 17; and
(c) CDR3 consisting of a sequence selected from the group consisting of the amino acid sequences for the CDR3 region of antibodies 13.1.2, 131, 170, 150, 095, 250, 139, 211, 124, 318, 342 and 333 as identified in SEQ ID NO: 138, 2, 4, 5, 7, 9, 10, 12, 13, 15, 16, and 17.

Further embodiments include a method of inhibiting cell proliferation associated with the expression of EGFRvIII, comprising treating cells expressing EGFRvIII with an effective amount of the conjugated antibody or fragment described above. The method can be performed in vivo, on a mammal, such as a human, suffering from a cancer involving epithelial cell proliferation, such as lung carcinoma, breast carcinoma, head & neck cancer, ovarian, colon, gastric, renal or prostate carcinoma or glioblastoma.

Other embodiments include an isolated polynucleotide molecule comprising a nucleotide sequence encoding a heavy chain amino acid sequence, or a fragment thereof, selected from the group consisting of the heavy chain amino acid sequence of antibodies 13.1.2, 131, 170, 150, 095, 250, 139, 211, 124, 318, 342, and 333 as identified in SEQ ID NO: 138, 2, 4, 5, 7, 9, 10, 12, 13, 15, 16, and 17, or an isolated polynucleotide molecule comprising a nucleotide sequence encoding a light chain amino acid sequence, or a fragment thereof, selected from the group consisting of the light chain amino acid sequence of antibodies 13.1.2, 131, 170, 150, 123, 095, 139, 250, 211, 318, 342, and 333, as identified in SEQ ID NO: 140, 19, 20, 21, 29, 23, 25, 26, 28, 33, 31 and 32.

Further embodiments include an article of manufacture comprising a container, a composition contained therein, and a package insert or label indicating that the composition can be used to treat cancer characterized by the expression of EGFRvIII, wherein the composition comprises a conjugated antibody as described above. Such cancers include a lung carcinoma, breast carcinoma, head & neck cancer, prostate, colon, gastric renal or ovarian carcinoma or glioblastoma. Also included is an assay kit for the detection of EGFRvIII in mammalian tissues or cells in order to screen for lung, breast, colon, gastric, renal, head and neck, prostate or ovarian carcinomas or glioblastomas, the EGFRvIII being an antigen expressed by epithelial cancers, the kit comprising an antibody that binds the antigen protein and means for indicating the reaction of the antibody with the antigen, if present. The antibody can be a labeled monoclonal antibody, or the antibody can be an unlabeled first antibody and the means for indicating the reaction comprises a labeled second antibody that is anti-immunoglobulin. The antibody that binds the antigen can be labeled with a marker selected from the group consisting of a fluorochrome, an enzyme, a Radionuclide and a radiopaque material. The antibody that binds to the antigen can be detected by a second labeled antibody. The antibody that binds the antigen can also bind to over-expressed wtEGFR. The kit can be used clinically for patient selection.

A further embodiment includes an antibody conjugated to a toxin, particularly DM1, which specifically recognizes the epitope of EGFRvIII containing the novel Gly residue.

Another embodiment includes an antibody, or variant thereof, conjugated to a toxin, particularly DM1, which binds to the recognition sequence EEKKGNYVVT (SEQ ID NO: 57).

In another embodiment, a method of treating a mammal having a tumor, the method comprising administering an anti-EGFRvIII antibody-drug conjugate to the mammal in need thereof at a dose of at least 0.1, 0.5, 1.0, 1.5, 2.0, 2.5, 3.0, 3.5. 4.0, 4.5, 5.0, 5.5, 6.0, 6.5, 7.0, 7.5, 8.0 8.5, 9.0, or 9.5 mg/kg to no more than 11.0 mg/kg is provided. In some embodiments the method provides administering the anti-EGFRvIII antibody-drug conjugate to the mammal in need thereof at a dose of at least 0.5 to 1 mg/kg, 0.1 to 2 mg/kg, 2 to 3 mg/kg, 3 to 4 mg/kg, 4 to 5 mg/kg, 5 to 6 mg/kg, 6 to 7 mg/kg, 7 to 8 mg/kg, 8 to 9 mg/kg 9-10 mg/kg, 1.5 to 2.5 mg/kg, 2.5 to 3.5 mg/kg, 3.5 to 4.5 mg/kg, 4.5 to 5.5 mg/kg or 5.5 to 6.5 mg/kg.

In this method of the invention administering step of the invention comprises administering the anti-EGFRvIII antibody-drug conjugate to the mammal intravenously, bolus injection, intracerebrally or by sustained release.

In this method of the invention the anti-EGFRvIII antibody-drug conjugate is administered at least twice every week, at least once every week, at least once every two weeks, at least once every three weeks or at least once every four weeks.

In this method of the invention, the tumor in the mammal, particularly a human, expresses EGFRvIII. In particular embodiments of this aspect of the invention the tumor is a lung carcinoma, breast carcinoma, colon carcinoma, gastric carcinoma, renal carcinoma, head & neck carcinoma, prostate carcinoma, ovarian carcinoma, glioblastoma, an anaplastic astrocytoma, astrocytoma or a tumor comprising a glial component, particularly glioblastoma, anaplastic astrocytoma, astrocytoma or a tumor comprising a glial component, more particularly a glioblastoma or an anaplastic astrocytoma, more particularly recurrent glioblastoma or recurrent anaplastic astrocytoma.

In one embodiment of this method the tumor in the mammal is oligodenroglioma, oligoastrocytoma, gliosarcoma, mixed glioma, pilocytic astrocytoma, pleomorphic xanthoastrocytoma, subependymal giant cell astrocytoma, astroblastoma, spongioblastoma, gliomatosis cerebri, or neuronal-glial tumors including gangliglioma, and anaplastic ganglioglioma

In this method of the invention the mammal is alive more than 3, more than 4, more than 5 or more than 6 months after administration of a first dose of anti-EGFRvIII antibody-drug conjugate.

In this method of the invention, the tumor in the mammal does not progress, after 3, after 4, after 5 or after 6 months from administration of a first dose of anti-EGFRvIII antibody-drug conjugate.

In this method of the invention, the mammal comprises a level of circulating tumor cells that is reduced as compared to the level of circulating tumor cells in the mammal before first administration of the anti-EGFRvIII antibody-drug conjugate.

In this method of the invention, the mammal comprises a level of exosomes characteristic of a tumor that is reduced as compared to the level of exosomes characteristic of a tumor in the mammal before the first administration of the anti-EGFRvIII antibody-drug conjugate. In this method of the invention, the tumor size in the mammal is reduced after administration of anti-EGFRvIII antibody-drug conjugate as compared the tumor size prior to administration of the first dose of anti-EGFRvIII antibody-drug conjugate.

In this method of the invention, the tumor size in mammal is decreased at least 1%, at least 5%, at least 10%, at least 15%, at least 20%, at least 25%, at least 30%, at least 40%, at least 50%, at least 60%, at least 70%, at least 80%, at least 90% or 100% as compared to the tumor size in the mammal prior to first administration of the anti-EGFRvIII antibody-drug conjugate as assessed by the Macdonald or RANO Criteria.

In this method of the invention, the mammal exhibits a complete or partial response as assessed by the MacDonald Criteria In this method of the invention, the mammal exhibits progression free survival of 6 month from the first administration of the anti-EGFRvIII antibody-drug conjugate as assessed by the Macdonald Criteria or RANO criteria.

In this aspect of the invention, the above methods result in an increased apparent diffusion coefficient from diffusion-weighted MRI (DWI) in a mammal as compared to the apparent diffusion coefficient detectable in the mammal prior to the first administration of the anti-EGFRvIII antibody-drug conjugate.

In this method of the invention, antibody of the anti-EGFRvIII antibody-drug conjugate is antibody 131 or antibody 13.1.2, particularly antibody 131. In one embodiment of this method of the invention, antibody of the anti-EGFRvIII antibody drug conjugate comprises a heavy chain variable region comprising the amino acid sequence of SEQ ID NO 2 and a light chain variable region comprising an amino acid sequence of SEQ ID NO 19.

In some embodiments of this method an anti-EGFRvIII antibody-drug conjugate is Ab 131-DM1 depicted in FIGS. 8A, 8B and 8D and comprises the heavy chain variable domain depicted in FIG. 8B and the light chain variable domain depicted in FIG. 8D.

In some embodiments of this method the anti-EGFRvIII antibody-drug conjugate is Ab 131-DM1 depicted in FIGS. 8A, 8B and 8D and comprises the full length heavy chain depicted in FIG. 8B and the full length light chain depicted in FIG. 8D.

In this method of the invention drug to which the anti-EGFRvIII antibody is conjugated is a radioactive isotopes, a chemotherapeutic agent, a toxins or a fragments or variants thereof, particularly to least one to 10, at least one to 5, or at least 3-5 maytansinoid DM1.

In this method of the invention drug is conjugated to the anti-EGFRvIII antibody via a non-cleavable linker group, particularly a thioether linker group, more particularly a succinimidyl-4-(N-maleimidomethyl)cyclohexane-1-carboxylate (SMCC). In the conjugated from the non-cleavable linker is MCC.

In a particular embodiment of this method an anti-EGFRvIII antibody comprising heavy chain variable region comprising the amino acid sequence of SEQ ID NO 2 and a light chain variable region comprising an amino acid sequence of SEQ ID NO 19 and the anti-EGFRvIII antibody conjugated to 3-5 maytansinoids by a MCC linker is used to treat the mammal having the tumor as described above.

In this method of the invention, the administration step is carried out prior to, in combination with or after treating the mammal having the tumor by applying surgery, applying radiationtherapy, applying whole brain radiation therapy in the primary setting, applying focal radiation therapy in the recurrent setting, administering temozolomide in the primary and recurrent setting, administering an anti-angigenic compound such as bevacizumab, administering irinotecan, administering PCV, procabazine, lomustine [CCNU], vincristine, implanting a Gliadel wafer (polifeprosan impregnated with BCNU), administering a tyrosine kinase inhibitor, administering a radio-sensitizing agent, administering a vaccine based therapy, administering an antibody drug conjugate, administering a Bi-specific T-cell engager in the primary or recurrent settings or administering a targeted drug to the mammal,

In an alternative embodiment of this method of the invention, the mammal has not previously been treated with an anti-angiogenic compound including bevacizumab. In this alternative embodiment, mammals that have not been treated with an anti-angiogenic compound prior to treatment with the anti-EGFRvIII antibody-drug conjugate of the invention have a more positive response than those previously treated with such an anti-angiogenic compound. In some embodiments the therapeutic molecule administered to the mammal in combination with the anti-EGFRvIII drug conjugate of the invention such as Ab 131-DM1, is another an anti-EGFRvIII therapeutic molecule such as an anti-EGFRvIII vaccine such as Rindopepimut, an anti-EGFRvIII antibody, another anti-EGFRvIII antibody drug conjugate, or an anti-EGFRvIII Bi-specific T-cell engager.

In some embodiments of this method of the invention the therapeutic molecule administered to the mammal in combination with the anti-EGFRvIII drug conjugate of the invention such as Ab 131-DM1, is an anti-EGFR therapeutic molecule such panitumumab, cetuximab, other anti-EGFR antibody, anti-EGFR vaccine, anti-EGFR antibody drug conjugate or anti-EGFR Bi-specific T-cell engager.

In some embodiments of this method of the invention the therapeutic molecule administered to the mammal in combination with the anti-EGFRvIII drug conjugate of the invention such as Ab 131-DM1, is an anti-Interleukin-6 therapeutic molecule such as an anti-Interleukin-6 antibody such as siltuximab, anti-Interleukin-6 receptor antibody such as tocilizumab, an anti-Interleukin-6 or anti-Interleukin-6 receptor antibody drug conjugate, or an anti-Interleukin-6 or anti-Interleukin-6 receptor Bi-specific T-cell engager.

In some embodiments of this method of the invention the therapeutic molecule administered to the mammal in combination with the anti-EGFRvIII drug conjugate of the invention such as Ab 131-DM1, is an anti-Interleukin-8 therapeutic molecule such as an anti-Interleukin-8 antibody, an anti-Interleukin-8 receptor antibody such as, an anti-Interleukin-8 or anti-Interleukin-8 receptor antibody drug conjugate, or an anti-Interleukin-8 or anti-Interleukin-8 receptor Bi-specific T-cell engager.

In some embodiments of this method of the invention the anti-EGFRvIII antibody drug conjugate of the invention, such as Ab 131-DM1 is administered prior to, in combination with or after administration of or more anti-EGFRvIII, anti-EGFR, anti-Interleukin-6, anti-Interleukin 6 receptor, anti-Interleukin-8 or anti-interleukn-8 receptor therapeutic molecules are administered to the mammal.

In some embodiments of this method of the invention the mammal is a human.

### BRIEF DESCRIPTION OF THE DRAWINGS

FIG. 1 is an alignment between wild type EGFR and EGFRvIII showing the 267 amino acid deletion and G substitution.

FIG. 2 is a diagram of the design of the EGFRvIII PEP3 14-mer peptide. In FIG. 2A, the N-terminal sequence of EGFRvIII with amino acids LEEKK (SEQ ID NO: 58) (1-5) that are identical to the N-terminal sequence of EGFR, followed by the unique Glysine residue, followed by amino acids that are identical to residues 273 through 280 in EGFR. FIG. 2B represents the amino acids of EGFR that are deleted in EGFRvIII (6-272).

FIGS. 3A-L provide sequences of antibodies of the invention. For each antibody provided, a nucleotide and amino acid sequence is provided for both a heavy chain and a light chain variable region. Accordingly, four sequences are provided for every antibody listed.

FIG. 4 is a table comparing the 13.1.2 antibody heavy chain regions to a particular germ line heavy chain region. “-”s indicate that the amino acid residue of the hybridoma heavy chain region is the same as the germ line for that particular position. Deviation from the germline is indicated by the appropriate amino acid residue.

FIG. 5 is a table comparing the 13.1.2 antibody light chain regions to a particular germ line light chain region. “-”s indicate that the amino acid residue of the hybridoma light chain region is the same as the germ line for that particular position. Deviation from the germline is indicated by the appropriate amino acid residue.

FIG. 6 is a table comparing various hybridoma derived antibody heavy chain regions to a particular germ line heavy chain region. “-”s indicate that the amino acid residue of the hybridoma heavy chain region is the same as the germ line for that particular position. Deviation from the germline is indicated by the appropriate amino acid residue.

FIG. 7 is a table comparing various hybridoma derived antibody light chain regions to a particular germ line light chain region. “-”s indicate that the amino acid residue of the hybridoma light chain region is the same as the germ line for that particular position. Deviation from the germline is indicated by the appropriate amino acid residue.

FIG. 8A is a schematic that depicts the structure of Ab 131-DM1 conjugate. FIG. 8B provides the amino acid sequence of the antibody heavy chain of the Ab 131-DMI conjugate, the variable domain is shaded and runs from the first amino acid through amino acid 123, while FIG. 8C provides the nucleic acid sequence encoding the antibody heavy chain. FIG. 8d provides the amino acid sequence of the antibody light chain of the Ab 131-DMI conjugate, the variable domain is shaded and runs from the first amino acid through amino acid 113, while FIG. 8E provides the nucleic acid sequence encoding the antibody light chain.

FIG. 9 is graph depicting the binding specificity of Anti-EGFRvIII antibody 131 (black dashed line), Ab 131-DM1 conjugate (solid black line), anti-EGFR antibody (gray dot dashed).

FIG. 10 are two plots depicting U251vIII cell growth inhibition as a function of toxin (DM1) equivalents (left panel) or Ab 131-DM1 conjugate equivalents (right panel).

FIG. 11 is a graph depicting the number of phospho-histone H3(+) cells detected in D317 subcutaneous xenografts (vertical axis) as a function of treatment with vehicle, AB 131-DM1 conjugate at 5.3 mg/kg, AB 131-DM1 conjugate at 17.8 mg/kg, or control conjugate.

FIG. 12 is a graph depicting tumor volume (vertical axis) as a function of time in U251vIII xenografts that had been treated with a single dose of control conjugate or Ab 131-DM1 conjugate at 1.7 mg/kg, 5.6 mg/k8 or 17 mg/kg.

FIG. 13 is a graph depicting tumor volume (vertical axis) as a function of time in D317 subcutaneous xenografts that had been treated with vehicle, control conjugate, anti-EGFRvIII antibody 131 or Ab 131-DM1 conjugate.

FIG. 14 is a graph depicting tumor volume (vertical axis) in D317 subcutaneous xenografts that had been treated with vehicle, control conjugate or a single dose of Ab 131-DM1 conjugate at 7.3 mg/kg, 14.6 mg/kg or 22 mg/kg.

### DETAILED DESCRIPTION OF THE PREFERRED EMBODIMENTS

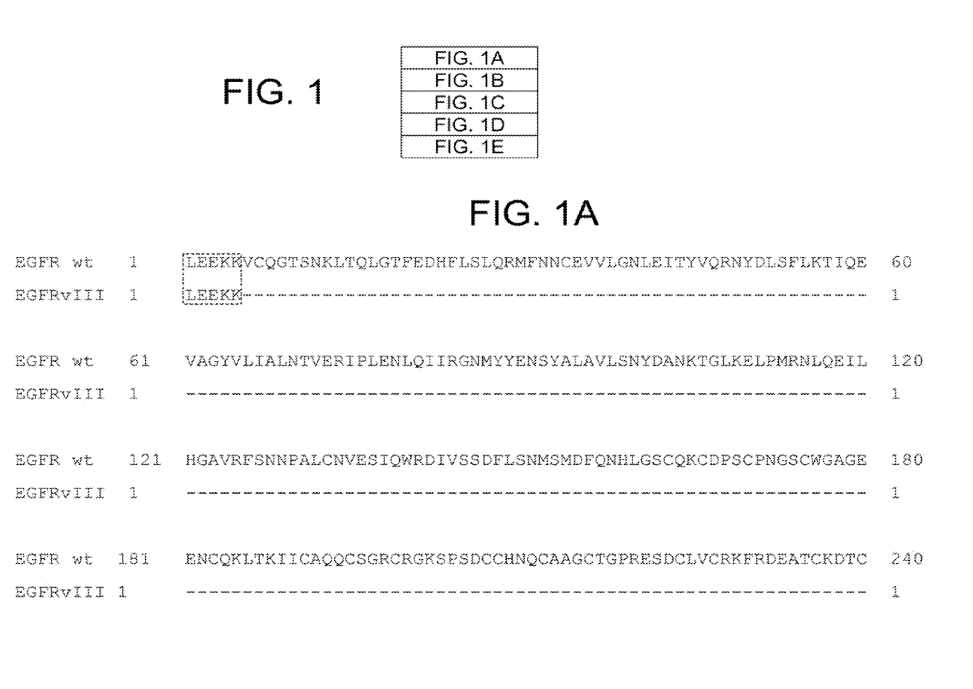

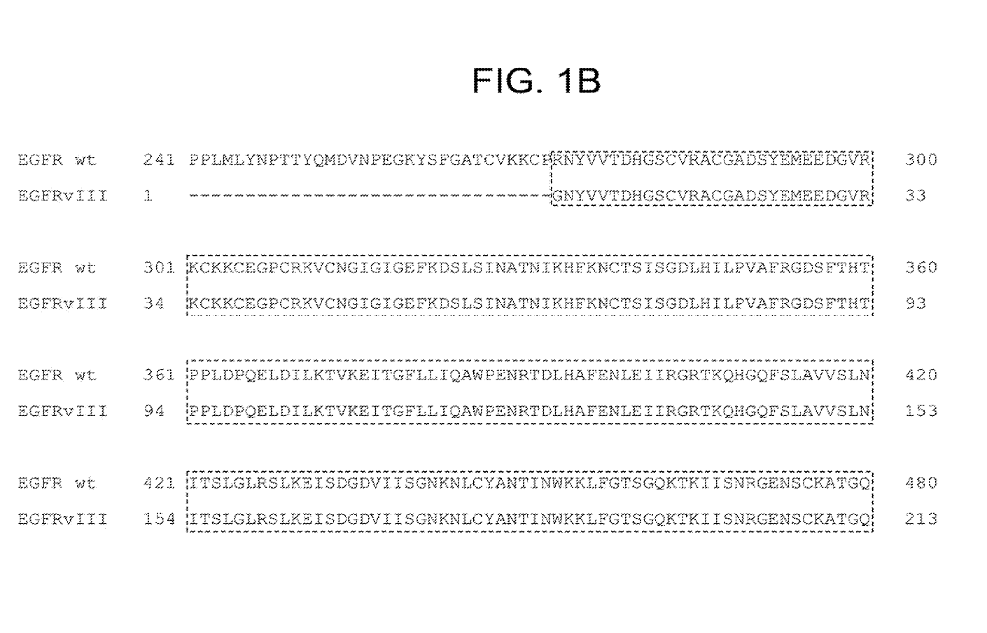

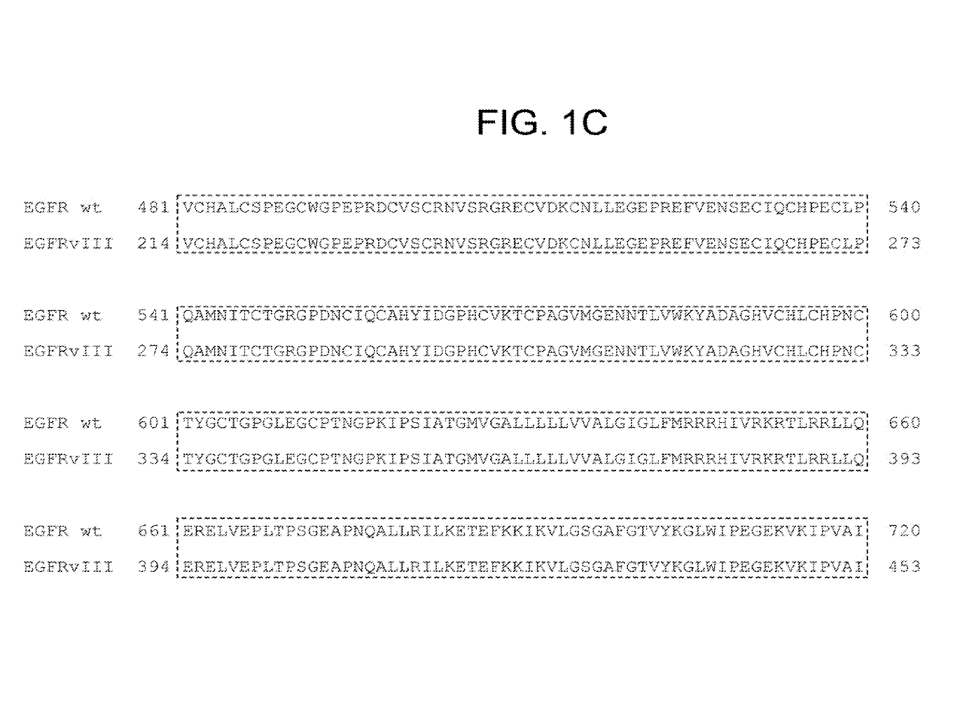

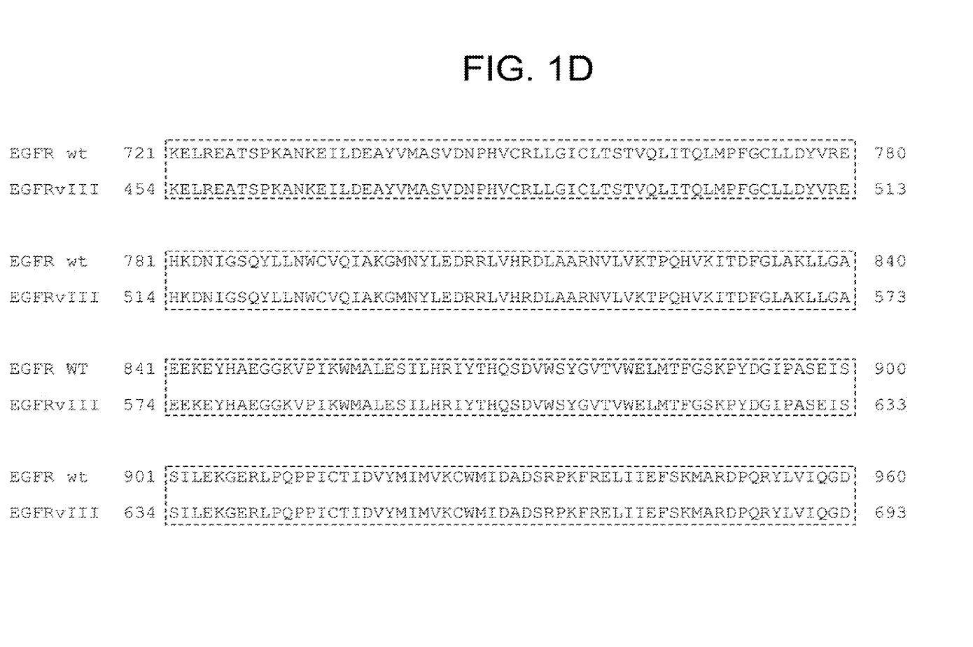

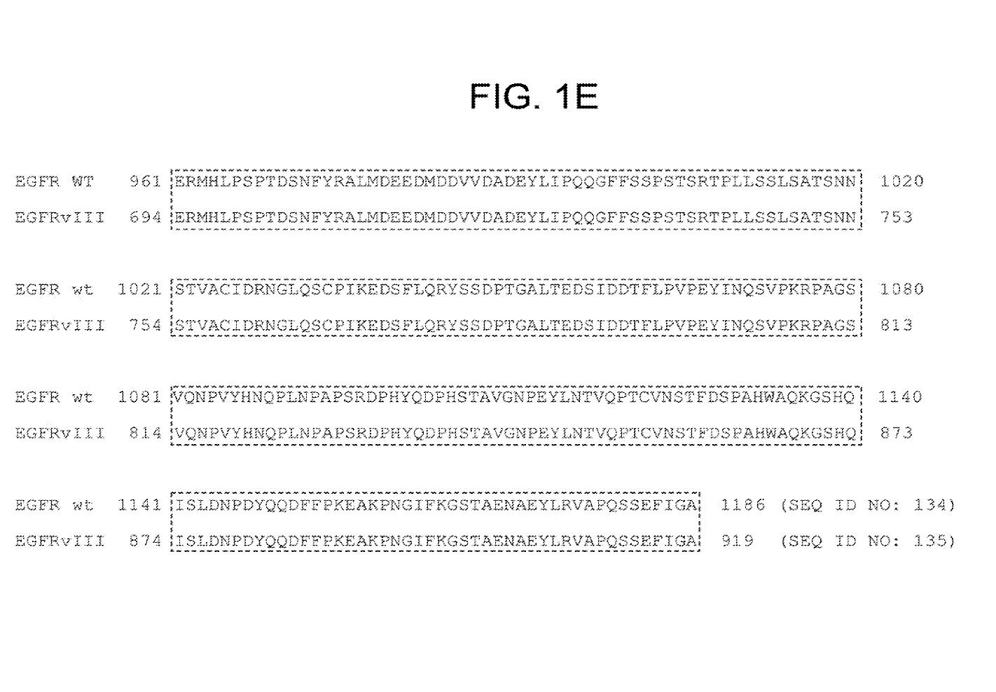

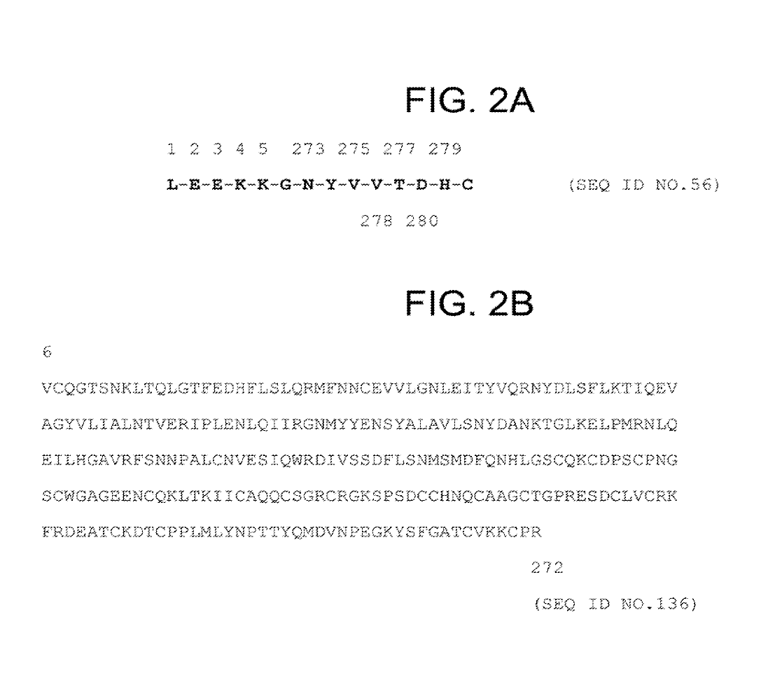

As discussed above, EGFRvIII is a deletion mutant of EGFR in which 267 amino acids in the extracellular domain of EGFr are deleted with a single amino acid substitution of Glycine at the junction. These features are shown in a sequence alignment between wild type EGFR and EGFRvIII in FIG. 1. In view of the amino acid substitution of Glycine at the junction of the deletion, it becomes theoretically possible to generate antibodies to the novel epitope present in EGFRvIII that is not present in wild type EGFR. Thus, a peptide for immunization and screening was designed, termed PEP3, as shown in FIG. 2 (Kuan et al. EGF mutant receptor vIII as a molecular target in cancer therapy. Endocr Relat Cancer. 8(2):83-96 (2001)). Such 14-mer peptide possesses the 5 n-terminal amino acids common to EGFRvIII and wild type EGFR, the unique Glycine junction site, and 8 amino acid residues contained in the conserved sequences between wild type EGFR (corresponding to residues 273-280) and EGFRvIII (corresponding to residues 7-14). In addition, glioblastoma cell and cells (B300.19 cells) transfected with the gene encoding EGFRvIII were also utilized for immunization and screening (sometimes referred to herein as B300 0.19/EGFRvIII transfectants).

In order to generate human antibodies against EGFRvIII, transgenic XenoMouse® mice were immunized with combinations of glioblastoma cells/EGFRvIII, B300.19/EGFRvIII cells, and peptides (PEP3) directed to the junction region in the novel extracellular domain represented in EGFRvIII as compared to wild type EGFR. B cells from immunized mice were isolated and either used to produce hybridomas followed by screening for binding to EGFRvIII or used directly in screening for binding to EGFRvIII using XenoMax™/SLAM™ technologies (Babcook et al. A novel strategy for generating monoclonal antibodies from single, isolated lymphocytes producing antibodies of defined specificities. Proc Natl Acad Sci USA. 93(15):7843-8 (1996), and U.S. Pat. No. 5,627,052). Antibodies identified that bound to EGFRvIII were screened in a series of assays to ascertain specific recognition of EGFRvIII. Through this process, panels of human monoclonal antibodies that bound to and were specific for EGFRvIII were generated, isolated, and characterized. Subsequent epitope mapping demonstrated unique but overlapping specificities. All antibodies were further evaluated in vitro for their ability to be internalized by cells for the purpose of delivering cytotoxic drugs to cells. Antibodies demonstrating efficient drug delivery were directly conjugated with a cytotoxic drug and examined for their ability to kill tumor cells expressing EGFRvIII in vitro and in vivo. These studies provide the basis for the next generation of antibody drug conjugates for treating cancer in patients whose tumor harbor specific genetic lesions.

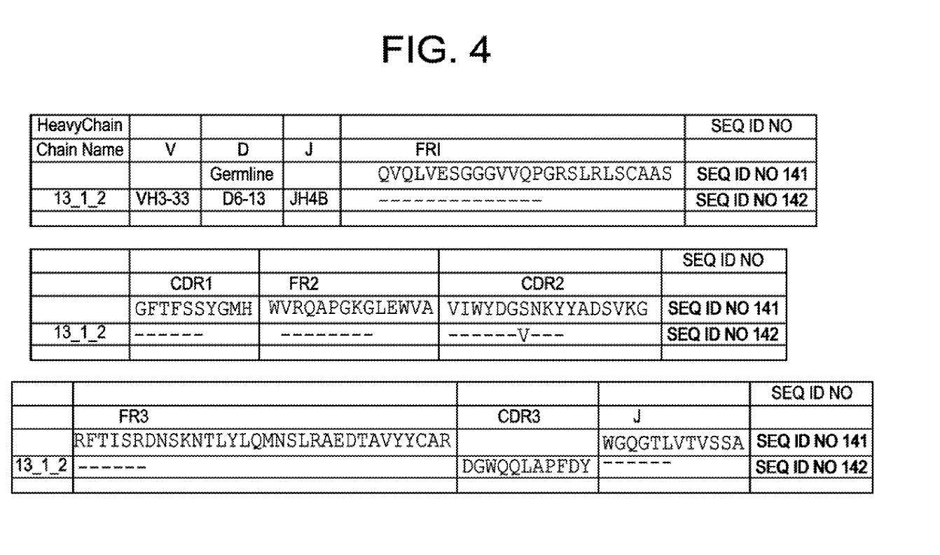

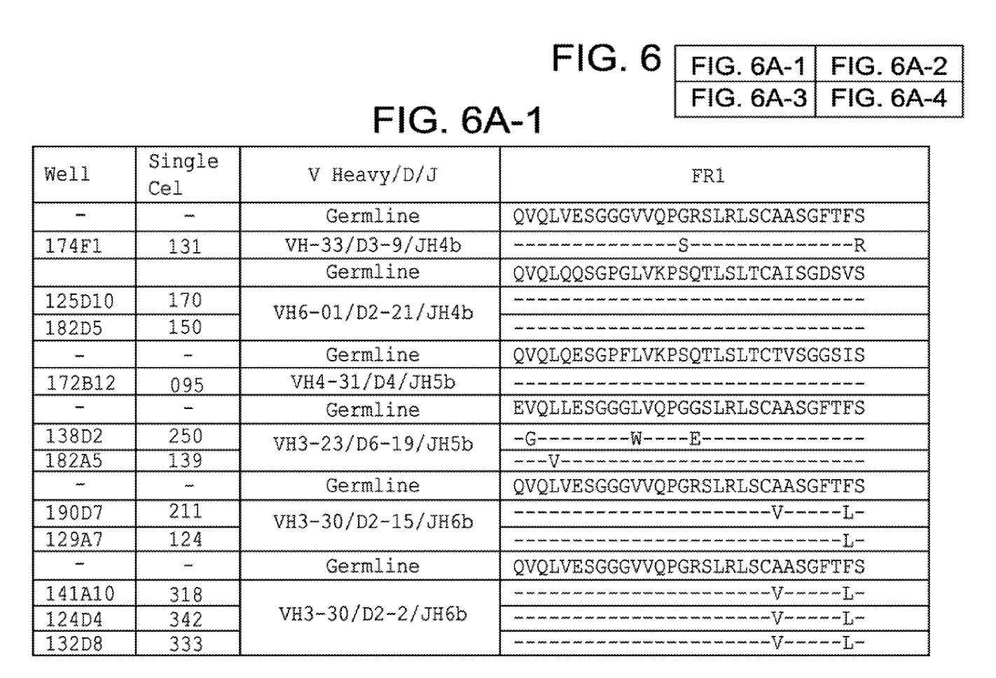

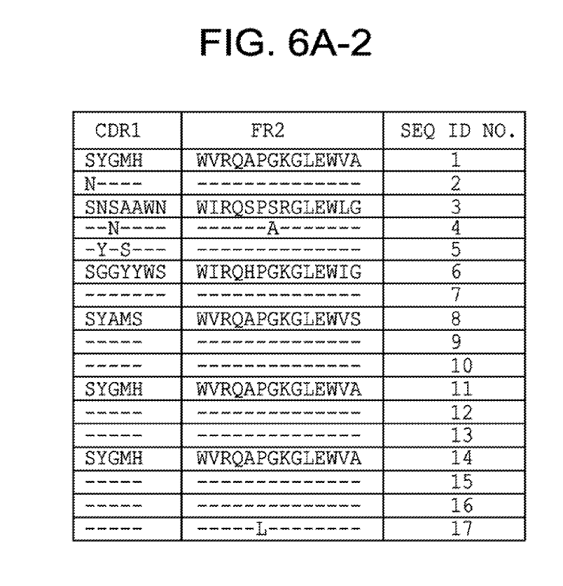

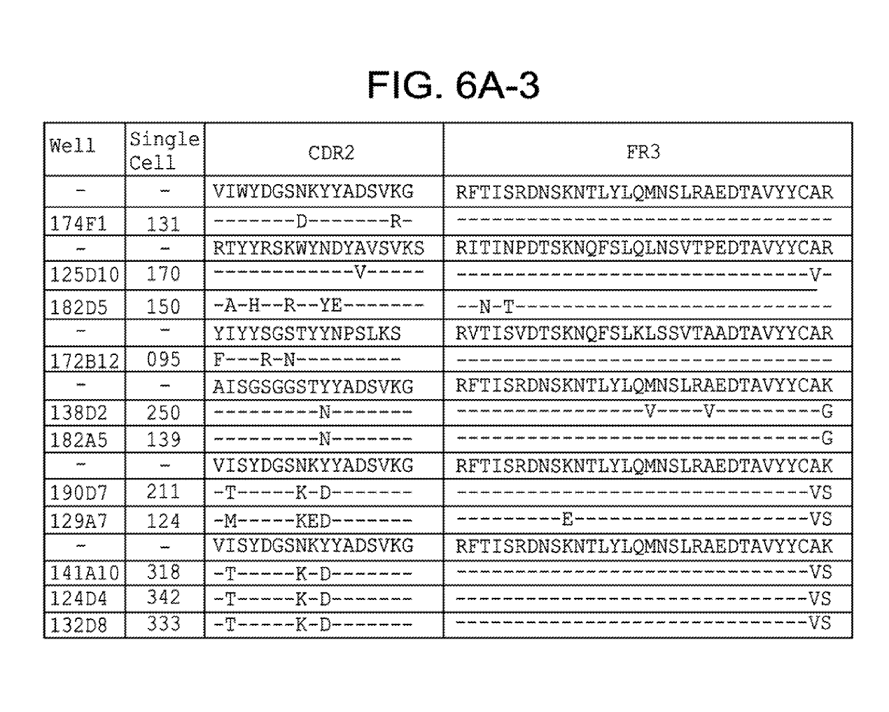

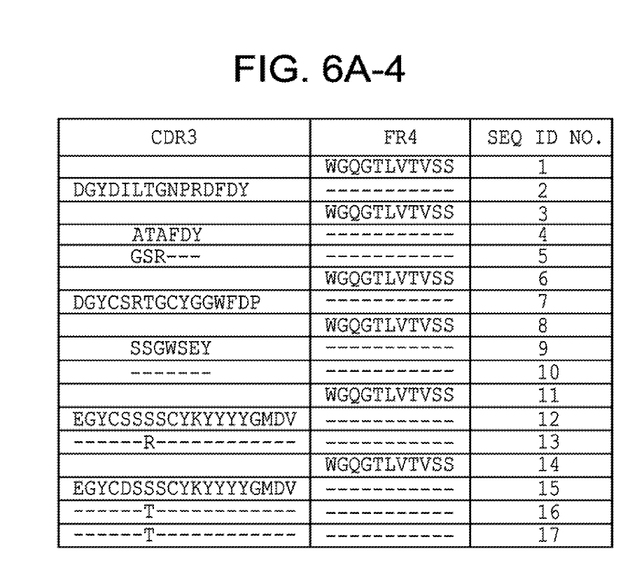

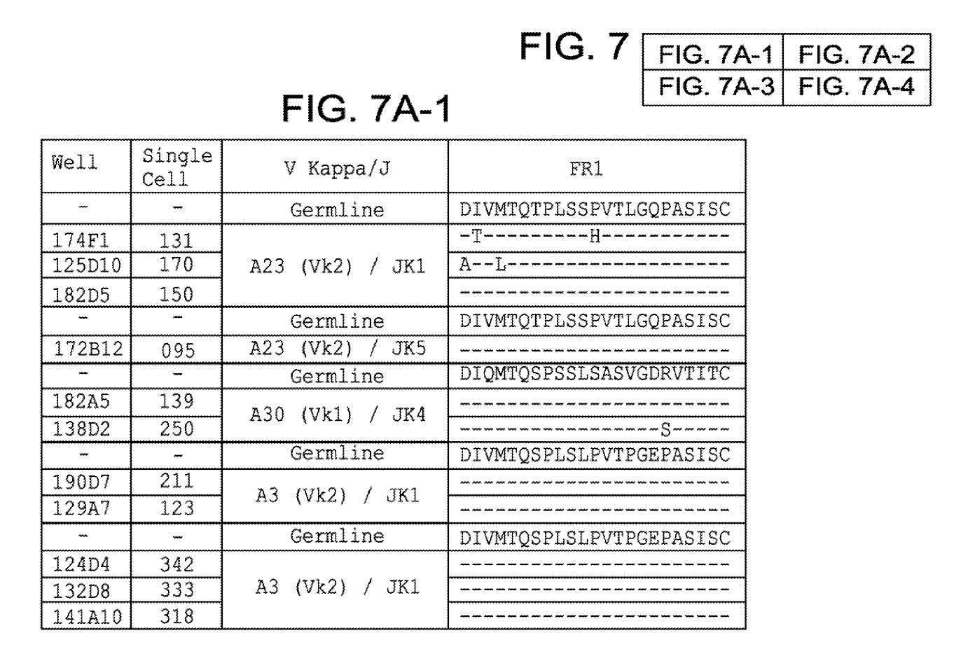

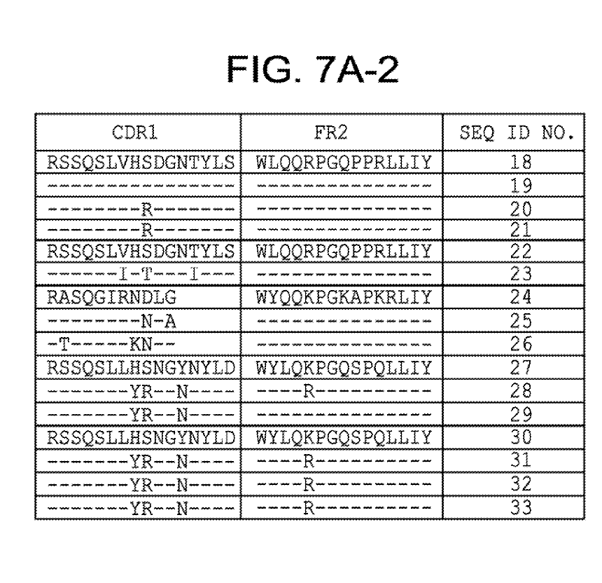

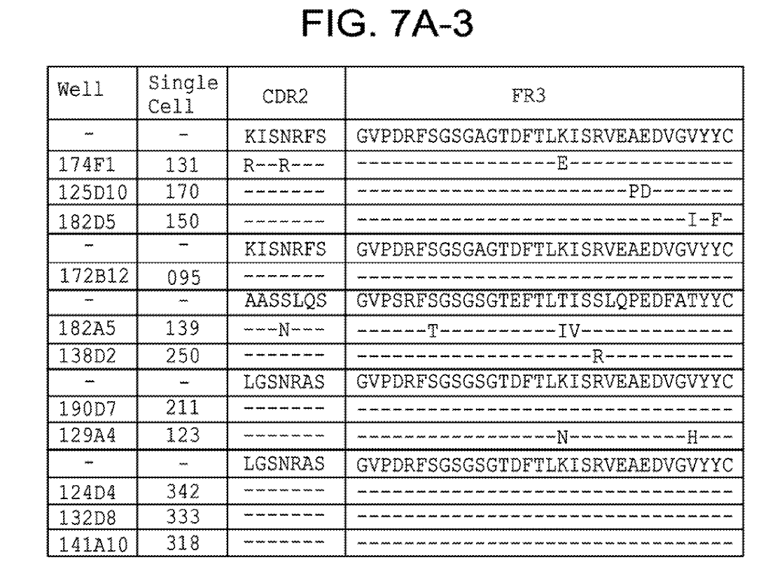

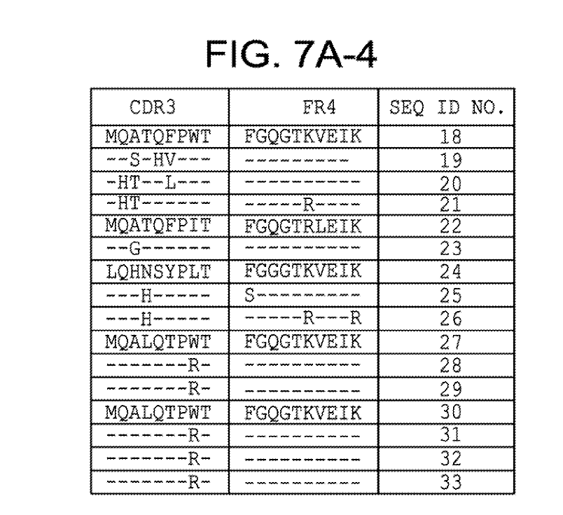

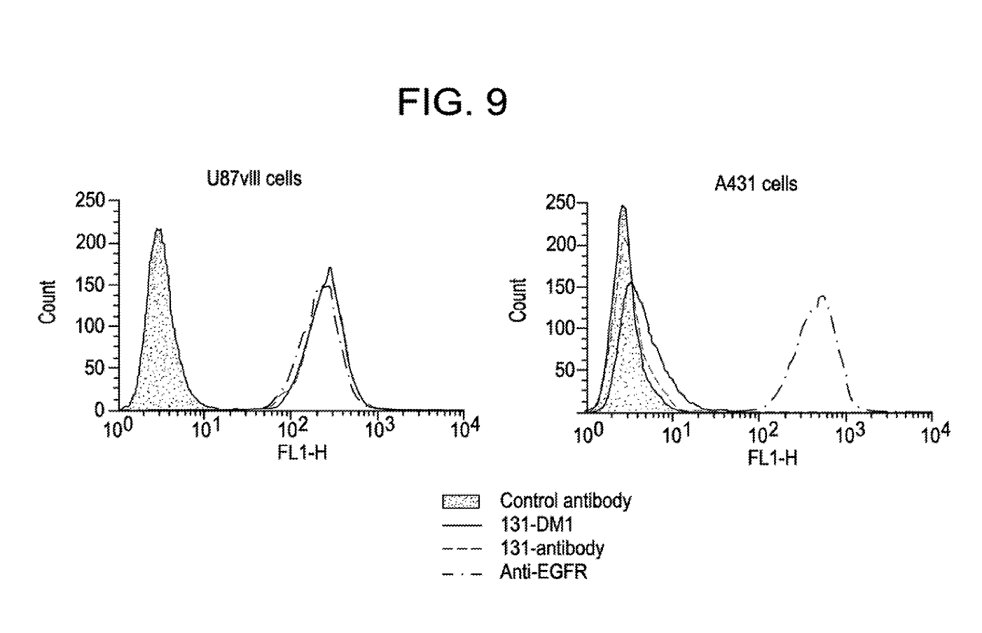

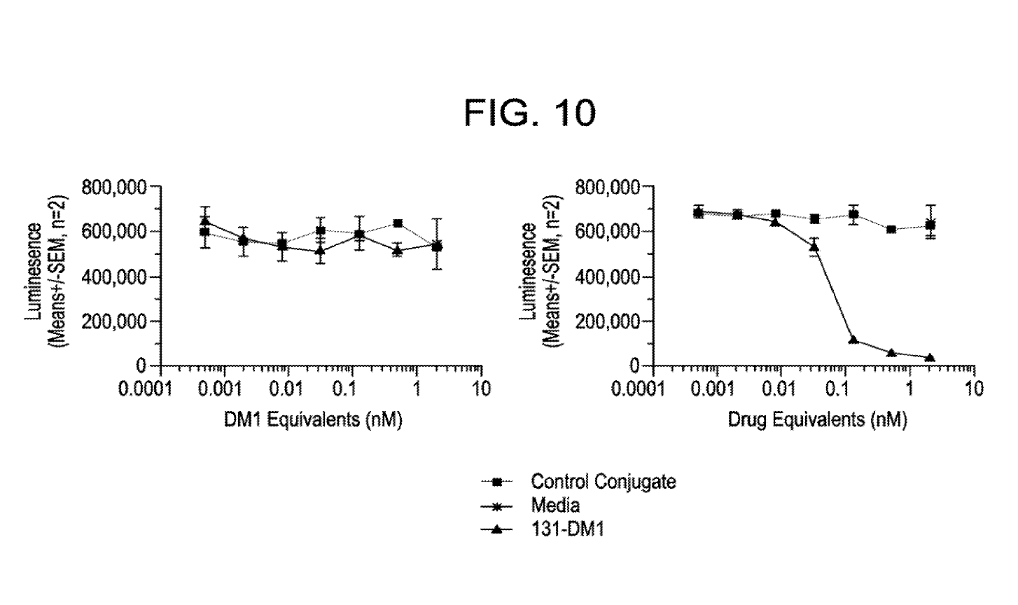

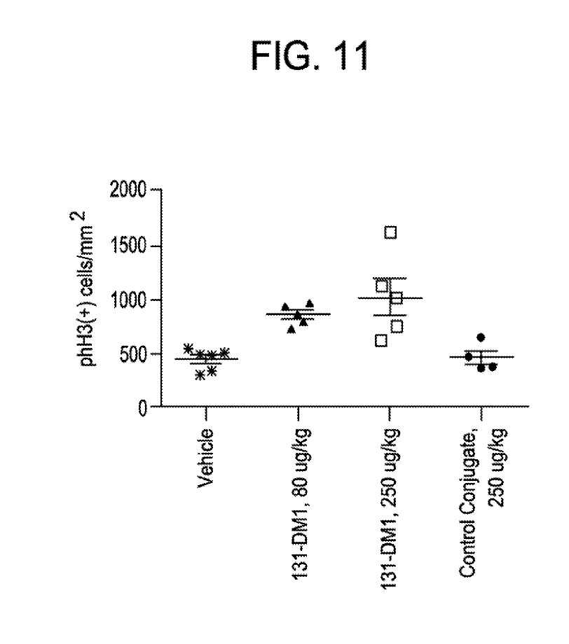

Through the processes described above, panels of fully human anti-EGFRvIII antibodies were generated. Using the hybridoma approach, several antibodies, including antibody 13.1, 13.2, 13.3, and 13.4 that were positive on ELISA for binding with the PEP3, were generated with limited cross-reactivity with wild type EGFR. Out of these, antibody 13.1 (and, particularly, its subclone 13.1.2) was selected for further research and development. Using the XenoMax approach a panel of antibodies, including antibody 131, 139, 250, and 095, were generated that were highly specific for binding with the pep3 oligonucleotide and had limited cross-reactivity with wild type EGFR. Of these, the 131 antibody has very interesting properties. The sequences for each of the antibodies are displayed in FIGS. 4-7 (SEQ ID NO: 1-33 and 141-144). A comparison of the sequences and binding abilities of the various antibodies was made and the results are displayed in FIGS. 4-10. As can be seen in FIGS. 9A-9L, and FIGS. 10A-10D antibodies 131, 139, and 13.1.2 all demonstrated superior selectivity for EGFRvIII expressing cells (H1477) as compared to ABX-EGF. Some of the results are shown in graph form in FIGS. 9M-9P, which demonstrates that at least two of the antibodies, 13.1.2 and 131 demonstrated superior specificity for EGFRvIII expressing cells compared to simply EGFRvIII cells. Additionally, several possible utilities for the antibodies of the current embodiment were examined; the results of which are shown in FIGS. 11-16. Finally, based on predicted structural models, variants of the antibodies were made in order to obtain antibodies with altered binding characteristics.

Further, antibodies of the invention are highly useful for the screening of other antibodies that bind to the same or similar epitopes. Antibodies of the invention can be utilized in cross competition studies for the elucidation of other antibodies that are expected to have the same or improved effects with respect to characteristics of the antigen-antibody complex that is formed.

Each of the 131 antibody and the 13.1.2 possessed very high affinities for EGFRvIII, were internalized well by cells, and appeared highly effective in cell killing when conjugated to toxins. Intriguingly, both of the antibodies, despite having been generated in different immunizations of XenoMouse mice, and utilizing different technologies, were derived from very similar germline genes. Based upon epitope mapping work, however, each of the antibodies appears to bind to slightly different epitopes on the EGFRvIII molecule and have slightly different residues on EGFRvIII that are essential for binding. These results indicate that the germline gene utilization is of importance to the generation of antibody therapeutics targeting EGFRvIII and that small changes can modify the binding and effects of the antibody in ways that allow for the further design of antibodies and other therapeutics based upon these structural findings.

Antibodies that bind to the same epitope as, or compete for binding with, the 13.1.2 and 131 antibodies are highly desirable. As discussed in more detail below, through Alanine scanning on SPOTs arrays important residues for binding of certain antibodies have been elucidated. Accordingly, antibodies that share critical binding residues are also highly desirable.

### DEFINITIONS

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y., 1989), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The term “Macdonald Criteria” shall mean the criteria set out in Macdonald D R, Cascino T L, Schold S C Jr, Cairncross J G. Response criteria for phase IIstudies of supratentorial malignant glioma. J Clin Oncol. 1990; 8:1277-1280.

The term “RANO Criteria” shall mean the criteria set out in Wen P Y, Macdonald D R, Reardon D A, and et al. Updated Response Assessment Criteria for High-Grade Gliomas: Response Assessment in Neuro-Oncology Working Group. J ClinOncol. 2010; 28: 1963-1972.

The term “complete response (CR)” shall mean the disappearance of all enhancing tumor on consecutive MRI imaging scans at least 4 weeks apart, off steroids and neurologically stable or improved.

The term “enhancing lesion” shall mean a lesion selected on the basis of size (lesions with the largest cross sectional area) and suitability for accurate repeated measurements.

The term “partial response (PR)” shall mean ≧50% reduction in size of enhancing tumor on consecutive MRI scans at least 4 weeks apart, steroids stable or reduced and neurologically stable or improved

The term “progression free survival (PFS)” is defined as the number of days from the date of first administration of Ab-131-DM1 to the date of radiological evidence of disease progression(date of MRI scan) or death, regardless of cause.

The term “positive response” shall mean reduction in tumor size, increased apparent diffusion coefficient, reduced circulating tumor cells, reduced circulating exomes associated with tumors as compared to these parameters in the mammal prior to the first administration of anti-EGFRvIII antibody conjugate. It also means progression free survival, complete response, and partial response as measured by the Macdonald or Rano Criteria. It also means increased survival.

The term BiTE or Bi-specific T-cell engager shall mean shall refer to fusion proteins comprising two single chain variable fragments (scFvs) of different antibodies in which one scFv binds to T cells vie the CD3 receptor and the other scFv binds to a molecue expressed on a tumor cell. Bi-specific T-cell inhibitors have been described in U.S. Pat. Nos. 7,352,641, 7,820,166, 8,076,450, 8,101,722, and 8,236,308.

The term “apparent diffusion coefficient” shall have the meaning set out in Chenevert, T. L. et al. Diffudion Magnet Resonance Imaging: an Early Surrogate Marker of Therapeutic Efficacy in Brain Tumors. Journal of the National Cancer Institute, Vol. 92, No. 24, Dec. 20, 2000.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g. free of murine proteins, (3) is expressed by a cell from a different species, or (4) does not occur in nature.

The term “polypeptide” is used herein as a generic term to refer to native protein, fragments, or analogs of a polypeptide sequence. Hence, native protein, fragments, and analogs are species of the polypeptide genus. Preferred polypeptides in accordance with the invention comprise the human heavy chain immunoglobulin molecules and the human kappa light chain immunoglobulin molecules, as well as antibody molecules formed by combinations comprising the heavy chain immunoglobulin molecules with light chain immunoglobulin molecules, such as the kappa light chain immunoglobulin molecules or lambda light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “variant” as used herein, is a polypeptide, polynucleotide, or molecule that differs from the recited polypeptide or polynucleotide, but only such that the activity of the protein is not detrimentally altered. There may be variants of epitopes. There may be variants of antibodies. In a preferred embodiment, the ability of a protein variant to bind to the epitope is not detrimentally altered. In one embodiment, the protein variant can bind with 10-500% of the ability of the wild type mAb. For example, the protein variant can bind with 10%, 50%, 110%, 500%, or greater than 500% of the ability of the wild type mAb. In one embodiment, the range of binding abilities between 10-500% is included. Binding ability may be reflected in many ways, including, but not limited to the kₐ, k_{d}, or K_{D} of the variant to an epitope. In one preferred embodiment, the epitope is one described in the present specification.

In one embodiment, variant antibodies can differ from the wild-type sequence by substitution, deletion or addition of five amino acids or fewer. Such variants may generally be identified by modifying one of the disclosed polypeptide sequences, and evaluating the binding properties of the modified polypeptide using, for example, the representative procedures described herein. In another embodiment, polypeptide variants preferably exhibit at least about 70%, more preferably at least about 90% and most preferably at least about 95% identity to the identified polypeptides. Preferably, the variant differs only in conservative substitutions and/or modifications. Variant proteins include those that are structurally similar and those that are functionally equivalent to the protein structures described in the present specification. In another embodiment, the protein is a variant if it is functionally equivalent to the proteins described in this specification, so long as the paratope of variant is similar to the paratopes described in the specification. In one embodiment, any substance with a shape that is similar to the paratope described in FIG. 17 is a variant. In one embodiment, any substance with a shape that is similar to the paratope described in FIG. 18 is a variant. In one embodiment, any substance that has a shape that is similar to the interaction surface described in FIGS. 19A and 19B is a variant.

In one embodiment, the antibody is a variant if the nucleic acid sequence can selectively hybridize to wild-type sequence under stringent conditions. In one embodiment, suitable moderately stringent conditions include prewashing in a solution of 5×SSC; 0.5% SDS, 1.0 mM EDTA (pH 8:0); hybridizing at 50° C.-65° C., 5×SSC, overnight or, in the event of cross-species homology, at 45° C. with 0.5×SSC; followed by washing twice at 65° C. for 20 minutes with each of 2×, 0.5× and 0.2×SSC containing 0.1% SDS. Such hybridizing DNA sequences are also within the scope of this invention, as are nucleotide sequences that, due to code degeneracy, encode an antibody polypeptide that is encoded by a hybridizing DNA sequence. The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence. Amino acids or nucleic acids with substantial identity to the wild-type protein or nucleic acid are examples of variants of the wild-type protein or nucleic acid.

As used herein, the twenty conventional amino acids and their abbreviations follow conventional usage. See Immunology-A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine. Polypeptides with substantial identity can be variants.

Variant proteins also include proteins with minor variations. As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated.

Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the antibodies described herein.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence. Analogs typically are at least 20 amino acids long, preferably at least 50 amino acids long or longer, and can often be as long as a full-length naturally-occurring polypeptide. Both fragments and analogs are forms of variants

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide. Peptide mimetics and peptidomimetics are both forms of variants.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “epitope” includes any protein determinant capable of specific binding to an immunoglobulin or T-cell receptor or otherwise interacting with a molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between the protein and the interacting molecule (such as an antibody) occur linearally along the primary amino acid sequence of the protein. In a conformational epitope, the points of interaction occur across amino acid residues on the protein that are separated from one another. An antibody is said to specifically bind an antigen when the dissociation constant is ≦1 μM, preferably ≦100 nM and more preferably ≦10 nM, and even more preferably ≦1 nM. Once a desired epitope on an antigen is determined, it is possible to generate antibodies to that epitope, e.g., using the techniques described in the present invention. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competively bind with one another, e.g., the antibodies compete for binding to the antigen. A high throughput process for “binning” antibodies based upon their cross-competition is described in International Patent Application No. WO 03/48731. As will be appreciated by one of skill in the art, practically anything to which an antibody can specifically bind could be an epitope. An epitope can comprises those residues to which the antibody binds. In one embodiment, the epitope is the EGFRvIII epitope. In one embodiment, the epitope comprises the sequence LEEKKGNYVVTD (SEQ ID NO: 59). In one embodiment, the epitope comprises the sequence EEKKGNYVVT (SEQ ID NO: 57). In one embodiment, the epitope comprises the sequence EKNY (SEQ ID NO: 60). In one embodiment, the epitope comprises the sequence EEKGN (SEQ ID NO: 61). One of skill in the art will appreciate that these need not be actually assembled in this order on a single peptide, rather, these are the residues that form the epitope which interacts with the paratope. As will be appreciated by one of skill in the art, the space that is occupied by a residue or side chain that creates the shape of a molecule helps to determine what an epitope is. Likewise, any functional groups associated with the epitope, van der Waals interactions, degree of mobility of side chains, etc. can all determine what an epitope actually is. Thus an epitope may also include energetic interactions.

The term “paratope” is meant to describe the general structure of a binding region that determines binding to an epitope. This structure influences whether or not and in what manner the binding region might bind to an epitope. Paratope can refer to an antigenic site of an antibody that is responsible for an antibody or fragment thereof, to bind to an antigenic determinant. Paratope also refers to the idiotope of the antibody, and the complementary determining region (CDR) region that binds to the epitope. In one embodiment, the paratope is the region of the antibody that is L1 10, L2 30, L3 50, H1 20, H2 40, and H3 60 in FIG. 17. In one embodiment, the paratope is the region of the antibody that comprises the CDR sequences in Example 16 for L1, L2, L3, H1, H2, and H3. In one embodiment, the paratope is the region of the antibody that is L1 110, L2 130, L3 150, H1 120, H2 140, and H3 160 in FIG. 18. In one embodiment, the paratope is the region of the antibody that comprises the CDR sequences in Example 18 for L1, L2, L3, H1, H2, and H3. In one embodiment, the paratope comprises the sequences listed in Example 18. In one embodiment, the paratope comprises the residues that interact with the epitope, as shown in FIG. 19A and FIG. 19B. The solid black structure is the peptide structure. In one embodiment, the paratope comprises residue Tyr172Arg of the 13.1.2 mAb. In one embodiment, the paratope of the 13.1.2 mAb comprises at least one residue selected from the group consisting of: Tyr 172Arg, Arg101Glu, Leu99Asn, Leu99His, Arg101Asp, Leu217Gln, Leu99Thr, Leu217Asn, Arg101Gln, and Asn35Gly. As will be appreciated by one of skill in the art, the paratope of any antibody, or variant thereof, can be determined in the manner set forth by the present application. Residues are considered “important” if they are predicted to contribute 10% of the binding energy. In one embodiment, residues are considered “important” if they are predicted to contribute 2% of the binding energy. In one embodiment, residues are considered “important” if they are predicted to contribute 50% of the binding energy. In one embodiment, residues are considered “important” if they are predicted to interact with the surface of the epitope, or the surface of the paratope. In one embodiment, residues are considered “important” if changing the residue results in a loss in binding.

The terms “specifically” or “preferentially” binds to, or similar phrases are not meant to denote that the antibody exclusively binds to that epitope. Rather, what is meant is that the antibody, or variant thereof, can bind to that epitope, to a higher degree than the antibody binds to at least one other substance to which the antibody is exposed to. In one embodiment, the specifically binding antibody will bind to the EGFRvIII protein with an affinity greater than (more tightly, or lower K_{D}) it will to the EGFR protein. For example, the specifically binding antibody will bind more tightly by at least a minimal increase to 1, 1-2, 2-5, 5-10, 10-20, 20-30, 30-50, 50-70, 70-90, 90-120, 120-150, 150-200, 200-300, 300-500, 500-1000 percent or more.

The shorthand of amino acid, number, amino acid, e.g., Leu217Gln, denotes a mutation at the numbered amino acid, from the first amino acid, to the second amino acid. Thus, Tyr172Arg would mean that while the wild type protein has a tyrosine at position 172, the mutant has an arginine at position 172.

The term “agent” is used herein to denote a chemical compound, a mixture of chemical compounds, a biological macromolecule, or an extract made from biological materials.

“Mammal” when used herein refers to any animal that is considered a mammal. Preferably, the mammal is human.

Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)₂ fragment in which the two arms of the antibody molecule remain linked and comprise two-antigen binding sites. The F(ab′)₂ fragment has the ability to crosslink antigen.

“Fv” when used herein refers to the minimum fragment of an antibody that retains both antigen-recognition and antigen-binding sites. These fragments can also be considered variants of the antibody.

“Fab” when used herein refers to a fragment of an antibody which comprises the constant domain of the light chain and the CH1 domain of the heavy chain.

The term “mAb” refers to monoclonal antibody.

The description of XenoMax method generated antibody sequences is coded as follows: “AB”-referring to antibody, “EGFRvIII”-referring to antibody's binding specificity, “X” referring to XenoMouse mouse derived, “G1”-referring to IgG1 isotype or “G2” referring to IgG2 isotype, the last three digits refer to the single cell number from which the antibody was derived, for example: AB-EGFRvIII-XG1-095 would be an antibody with binding specificity to EGFRvIII from XenoMouse mouse of a IgG1 isotype and cell number 95.

The term “SC” refers to single cell and a particular XenoMax method derived antibody may be referred to as SC followed by three digits, or just three digits, referring to the single cell number from which the antibody was derived herein.

The description of hybridoma derived antibody sequences is coded as follows: “AB”-referring to antibody, “EGFRvIII”-refers to the antibody's binding specificity, “X” refers to XenoMouse mouse derived, “G1”-refers to IgG1 isotype or “G2” refers to IgG2 isotype, “K” refers to kappa, “L’ refers to lambda. The last three digits referring to the clone from which the antibody was derived, for example: AB-EGFRvIII-XG1K-13.1.2

“Label” or “labeled” as used herein refers to the addition of a detectable moiety to a polypeptide, for example, a radiolabel, fluorescent label, enzymatic label chemiluminescent labeled or a biotinyl group. Radioisotopes or radionuclides may include ³H, ¹⁴C, ¹⁵N, ³⁵S, ⁹⁰Y, ⁹⁹Tc, ¹¹¹In, ¹²⁵I, ¹³¹I, fluorescent labels may include rhodamine, lanthanide phosphors or FITC and enzymatic labels may include horseradish peroxidase, β-galactosidase, luciferase, alkaline phosphatase.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), (incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, 99%, and 99.9%. Most preferably, the object species is purified to essential homogeneity (contaminant species cannot be detected in the composition by conventional detection methods) wherein the composition consists essentially of a single macromolecular species.

The term “patient” includes human and veterinary subjects.

The term “SLAM® Technology” refers to the “Selected Lymphocyte Antibody Method” (Babcook et al., Proc. Natl. Acad. Sci. USA, i93:7843-7848 (1996), and Schrader, U.S. Pat. No. 5,627,052, both of which are incorporated by reference in their entirety).

The term “XenoMax™” refers to the use of SLAM Technology with XenoMouse® mice (as described below).

### Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992). Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

In addition to the general structural aspects of antibodies, the more specific interaction between the paratope and the epitope may be examined through structural approaches. In one embodiment, the structure of the CDRs form a paratope, through which an antibody is able to bind to an epitope. The structure of such a paratope may be determined in a number of ways. Traditional structural examination approaches may be used, such as NMR or x-ray crystalography. These approaches may examine the structure of the paratope alone, or while it is bound to the epitope. Alternatively, molecular models may be generated in silico. A structure can be generated through homology modeling, aided with a commercial package, such as Insightll modeling package from Accelrys (San Diego, Calif.). Briefly, one can use the sequence of the antibody to be examined to search against a database of proteins of known structures, such as the Protein Data Bank. After one identifies homologous proteins with known structures, these homologous proteins are used as modeling templates. Each of the possible templates can be aligned, thus producing structure based sequence alignments among the templates. The sequence of the antibody with the unknown structure can then be aligned with these templates to generate a molecular model for the antibody with the unknown structure. As will be appreciated by one of skill in the art, there are many alternative methods for generating such structures in silico, any of which may be used. For instance, a process similar to the one described in Hardman et al., issued U.S. Pat. No. 5,958,708 employing QUANTA (Polygen Corp., Waltham, Mass.) and CHARM (Brooks, B. R., Bruccoleri, R. E., Olafson, B. D., States, D. J., Swaminathan, S. and Karplus, M., 1983, J. Comp. Chem, 4:187) may be used (hereby incorporated in its entirety by reference).

Not only is the shape of the paratope important in determining whether and how well a possible paratope will bind to an epitope, but the interaction itself, between the epitope and the paratope is a source of great information in the design of variant antibodies. As appreciated by one of skill in the art, there are a variety of ways in which this interaction can be studied. One way is to use the structural model generated, perhaps as described above, and then to use a program such as InsightII (Accelrys, San Diego, Calif.), which has a docking module, which, among other things, is capable of performing a Monte Carlo search on the conformational and orientational spaces between the paratope and its epitope. The result is that one is able to estimate where and how the epitope interacts with the paratope. In one embodiment, only a fragment, or variant, of the epitope is used to assist in determining the relevant interactions. In one embodiment, the entire epitope is used in the modeling of the interaction between the paratope and the epitope. As will be appreciated by one of skill in the art, these two different approaches have different advantages and disadvantages. For instance, using only a fragment of the epitope allows for a more detailed examination of the possible variations of each side chain, without taking huge amounts of time. On the other hand, by using only a fragment of the epitope, or simply the epitope instead of the entire protein, it is possible that the characteristics of the epitope fragment may not be the same as the characteristics for the whole epitope, thus possibly increasing the risk of being mislead during the computational modeling. In one embodiment, both approaches are used to a limited extent, in order to cross check the results. In a preferred embodiment, if a variant of an epitope is used, it will be optimized so that the variant of the epitope comprises the most important residues of the epitope. The identity of the most important residues can be determined in any number of ways, for instance as described in Examples 4 and 14 of the present specification.

Through the use of these generated structures, one is able to determine which residues are the most important in the interaction between the epitope and the paratope. Thus, in one embodiment, one is able to readily select which residues to change in order to alter the binding characteristics of the antibody. For instance, it may be apparent from the docking models that the side chains of certain residues in the paratope may sterically hinder the binding of the epitope, thus altering these residues to residues with smaller side chains may be beneficial. One can determine this in many ways. For example, one may simply look at the two models and estimate interactions based on functional groups and proximity. Alternatively, one may perform repeated pairings of epitope and paratope, as described above, in order to obtain more favorable energy interactions. One can also determine these interactions for a variety of variants of the antibody to determine alternative ways in which the antibody may bind to the epitope. One can also combine the various models to determine how one should alter the structure of the antibodies in order to obtain an antibody with the particular characteristics that are desired.

The models determined above can be tested through various techniques. For example, the interaction energy can determined with the programs discussed above in order to determine which of the variants to further examine. Also, coulumbic and van der Waals interactions are used to determine the interaction energies of the epitope and the variant paratopes. Also site directed mutagenesis is used to see if predicted changes in antibody structure actually result in the desired changes in binding characteristics. Alternatively, changes may be made to the epitope to verify that the models are correct or to determine general binding themes that may be occurring between the paratope and the epitope.

The above methods for modeling structures can be used to determine what changes in protein structure will result in particular desired characteristics of an antibody. These methods can be used to determine what changes in protein structure will not result in the desired characteristics.

As will be appreciated by one of skill in the art, while these models will provide the guidance necessary to make the antibodies and variants thereof of the present embodiments, it may still be desirable to perform routine testing of the in silico models, perhaps through in vitro studies. In addition, as will be apparent to one of skill in the art, any modification may also have additional side effects on the activity of the antibody. For instance, while any alteration predicted to result in greater binding, may induce greater binding, it may also cause other structural changes which might reduce or alter the activity of the antibody. The determination of whether or not this is the case is routine in the art and can be achieved in many ways. For Example, the activity can be tested through an ELISA test, as in Example 21. Alternatively, the samples can be tested through the use of a surface plasmon resonance device.

### Antibodies Binding, and Variant Antibodies for Superior Binding

In one embodiment, the models described above are used to increase the binding ability of the antibody to its epitope. The antibody can bind to the epitope more readily, and thus have a higher association constant (kₐ). Alternatively, the antibody may dissociate from the epitope slower, and thus have a lower dissociation constant (k_{d}), or the K_{D} of the epitope-paratope interaction can be smaller in value, thus making the extent of the binding between the epitope and paratope higher.

In some embodiments, the variant antibodies are designed to bind with the opposite characteristics. That is, the antibodies do not bind as tightly or perhaps as quickly.

In other embodiments, the variant antibodies are not different in their K_{D} from the wild-type antibodies, but the variant antibodies are more specific for a particular epitope. This may mean that the paratopes of the designed antibodies have a lower risk of binding to other epitopes. The antibodies can have other characteristics that have been altered. For example, a variant may be more immune to nonspecific antibody binding or may stay solvated in solution even when the antibody is present in high concentrations. Such a variant may be present in the discussed antibodies. For instance, while the higher concentrations of some variant antibodies examined below resulted in slower binding components in Biacore experiments, for instance 13.1.2 mAb, certain variants did not exhibit this slower component, even at the same concentrations, L217N-2.1, for example.

The variants predicted by the models determined above can be created and then tested to determine if they actually bind with the desired characteristics. Mutants with a greater total interaction energy with the epitope can be selected for further testing. The interaction energy can be determined in a number of ways, one of which is described above.

These variants can be tested in a number of ways. Exemplary options include and are not limited to KinExA (e.g., Lackie, Issued U.S. Pat. No. 5,372,783, Dec. 13, 1994, herein incorporated in its entirety by reference)(Sapidyne Instruments Inc., ID, Boise), surface plasmon resonance (SPR)(e.g., BIACORE™ Biacore, Inc., Pistcataway, N.J.), stopped-flow fluorescence, resonant mirror, and fluorescence polarization. Many of these options are able to not only record the data, but also provide ready means for fitting the data to various theoretical curves and thus determine the kₐ, k_{d}, and K_{D}, as well as other properties. It is important to note that the fitting of these curves to the resulting data is not without the possibility for some variation. Because of this, the relevant association, dissociation, and equilibrium constants can be looked at, not only through these curve fitting mechanisms, but also in direct comparison with each other, and in light of the knowledge of one of skill in the art.

### Human Antibodies and Humanization of Antibodies

Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the utilization of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies are expected to minimize the immunogenic and allergic responses intrinsic to mouse or mouse-derivatized mAbs and thus to increase the efficacy and safety of the administered antibodies. The use of fully human antibodies can be expected to provide a substantial advantage in the treatment of chronic and recurring human diseases, such as inflammation, autoimmunity, and cancer, which require repeated antibody administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with our generation of the first XenoMouse mouse strains, as published in 1994. (See Green et al. Nature Genetics 7:13-21 (1994)) The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. Id. The human Ig containing YACs proved to be compatible with the mouse system for both rearrangement and expression of antibodies and were capable of substituting for the inactivated mouse Ig genes. This was demonstrated by their ability to induce B-cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620, filed Dec. 3, 1996, the disclosures of which are hereby incorporated by reference.

The production of the XenoMouse mice is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, 08/430, 938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and 08/759,620, filed Dec. 3, 1996 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998). See also European Patent No., EP 0 463 151 B 1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000, WO 03/47336. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023.010 to Krimpenfort and Berns, U.S. Pat. Nos. 5,612,205, 5,721,367, and U.S. Pat. No. 5,789,215 to Berns et al., and U.S. Pat. No. 5,643,763 to Choi and Dunn, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996, 5,698,767, and 5,958,765. Human anti-mouse antibody (HAMA) responses have led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against EGFRvIII in order to vitiate concerns and/or effects of HAMA or HACA response.

### Conjugated Antibody Therapeutics

As discussed herein, the function of the EGFRvIII antibody appears important to at least a portion of its mode of operation. By function, it is meant, by way of example, the activity of the EGFRvIII antibody in operation with EGFRvIII. Accordingly, in certain respects, it may be desirable in connection with the generation of antibodies as therapeutic candidates against EGFRvIII that the antibodies be capable of fixing complement and recruiting cytotoxic lymphocytes thus participating in CDC and ADCC. There are a number of isotypes of antibodies that are capable of the same, including, without limitation, the following: murine IgM, murine IgG2a, murine IgG2b, murine IgG3, human IgM, human IgG1, human IgG3, and human IgA. Also, it may be desirable in connection with the generation of antibodies as therapeutic candidates against EGFRvIII that the antibodies be capable of activating antibody-dependent cellular cytotoxicity (ADCC), through engagement of Fc receptors on effectors cells such as natural killer (NK) cells. There are a number of isotypes of antibodies that are capable of ADCC, including, without limitation, the following: murine IgG2a, murine IgG2b, murine IgG3, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see e.g., U.S. Pat. No. 4,816,397) and cell-cell fusion techniques (see e.g., U.S. Pat. Nos. 5,916,771 and 6,207,418), among others.

In the cell-cell fusion technique, a myeloma or other cell line is prepared that possesses a heavy chain with any desired isotype and another myeloma or other cell line is prepared that possesses the light chain. Such cells can, thereafter, be fused and a cell line expressing an intact antibody can be isolated.

By way of example, certain anti-EGFRvIII antibodies discussed herein are human anti-EGFRvIII IgG1 antibodies. If such antibody possessed desired binding to the EGFRvIII molecule, it could be readily isotype switched to generate a human IgM, human IgG3, or human IgGA while still possessing the same variable region (which defines the antibody's specificity and some of its affinity). Such molecules, including IgG1, would then be capable of fixing complement and participating in CDC, and, if comprising and IgG1 or IgG3 constant region, such molecules would also be capable of participating in antibody-dependent cellular cytotoxicity (ADCC) through recruiting cytotoxic lymphocytes.

Accordingly, as antibody candidates are generated that meet desired “structural” attributes as discussed above, they can generally be provided with at least certain of the desired “functional” attributes through isotype switching.

### Design and Generation of Other Therapeutics

Based on the activity of the antibodies that are produced and characterized herein with respect to EGFRvIII, the design of other therapeutic modalities beyond antibody moieties is facilitated. Such modalities include, without limitation, advanced antibody therapeutics, such as bispecific antibodies, immunotoxins, and radiolabeled therapeutics, generation of peptide therapeutics, gene therapies, particularly intrabodies, antisense therapeutics, and small molecules.

In connection with the generation of advanced antibody therapeutics, where complement fixation and recruitment of cytoxic lymphocytes is a desirable attribute, it is possible to enhance cell killing through the use of bispecifics, immunotoxins, or radiolabels, for example.

For example, in connection with bispecific antibodies, bispecific antibodies can be generated that comprise (i) two antibodies one with a specificity to EGFRvIII and another to a second molecule that are conjugated together, (ii) a single antibody that has one chain specific to EGFRvIII and a second chain specific to a second molecule, or (iii) a single chain antibody that has specificity to EGFRvIII and the other molecule. Such bispecific antibodies can be generated using techniques that are well known for example, in connection with (i) and (ii) see e.g., Fanger et al. Immunol Methods 4:72-81 (1994) and Wright and Harris, supra. and in connection with (iii) see e.g., Traunecker et al. Int. J. Cancer (Suppl.) 7:51-52 (1992). In each case, the second specificity can be made to the Fc chain activation receptors, including, without limitation, CD16 or CD64 (see e.g., Deo et al. 18:127 (1997)) CD3 (Micromet's BiTE technology) or CD89 (see e.g., Valerius et al. Blood 90:4485-4492 (1997)). Bispecific antibodies prepared in accordance with the foregoing would be likely to kill cells expressing EGFRvIII, and particularly those cells in which the EGFRvIII antibodies of the invention are effective.

In connection with immunotoxins, antibodies can be modified to act as immunotoxins utilizing techniques that are well known in the art. See e.g., Vitetta Immunol Today 14:252 (1993). See also U.S. Pat. No. 5,194,594. In connection with the preparation of radiolabeled antibodies, such modified antibodies can also be readily prepared utilizing techniques that are well known in the art. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), 5,648,471, and 5,697,902. Each of immunotoxins and radiolabeled molecules would be likely to kill cells expressing EGFRvIII, and particularly those cells in which the antibodies described herein are effective.

The antibodies can be designed to bind more quickly, or to dissociate more slowly from the epitope, and thus the antibodies themselves can be designed therapeutics. The altered characteristics of the antibodies can be used, for example, in the administration of a therapeutic to a patient.

### Therapeutic Immunoconjugates

As will be appreciated, antibodies conjugated to drugs, toxins, or other molecules (herein referred to interchangeably as immunoconjugates, immunotoxins or antibody drug conjugates (“ADC”)) are highly useful in the targeted killing of cells that express a molecule that can be specifically bound by a specific binding molecule, such as an antibody. T As discussed above, EGFRvIII is not known to be expressed on any normal tissues. Further, EGFRvIII shows significant expression in numerous human tumors. Accordingly, EGFRvIII is a highly attractive molecule for targeting with an immunotoxin.

Many reports have appeared on the attempted specific targeting of tumor cells with monoclonal antibody-drug conjugates (Sela et al. in Immunoconjugates 189-216 (C. Vogel, ed. 1987); Ghose et al, in Targeted Drugs 1-22 (E. Goldberg, ed. 1983); Diener et al, in Antibody Mediated Delivery Systems 1-23 (J. Rodwell, ed. 1988); Pietersz et al, in Antibody Mediated Delivery Systems 25-53 (J. Rodwell, ed. 1988); Bumol et al, in Antibody Mediated Delivery Systems 55-79 (J. Rodwell, ed. 1988). Cytotoxic drugs such as methotrexate, daunorubicin, doxorubicin, vincristine, vinblastine, melphalan, mitomycin C, and chlorambucil have been conjugated to a variety of murine monoclonal antibodies. In some cases, the drug molecules were linked to the antibody molecules through an intermediary carrier molecule such as serum albumin (Garnett et al. Cancer Res. 46:2407-2412 (1986); Ohkawa et al. Cancer Immumol. Immunother. 23:81-86 (1986); Endo et al. Cancer Res. 47:1076-1080 (1980)), dextran (Hurwitz et al. Appl. Biochem. 2:25-35 (1980); Manabi et al. Biochem. Pharmacol. 34:289-291 (1985); Dillman et al. Cancer Res. 46:4886-4891 (1986); Shoval et al. Proc. Natl. Acad. Sci. 85: 8276-8280 (1988)), or polyglutamic acid (Tsukada et al. J. Natl. Canc. Inst. 73:721-729 (1984); Kato et al. J. Med. Chem. 27:1602-1607 (1984); Tsukada et al. Br. J. Cancer 52:111-116 (1985)).

A wide array of linker technologies has been employed for the preparation of such immunoconjugates and both cleavable and non-cleavable linkers have been investigated. In most cases, the full cytotoxic potential of the drugs could only be observed, however, if the drug molecules could be released from the conjugates in unmodified form at the target site.

One of the cleavable linkers that has been employed for the preparation of antibody-drug conjugates is an acid-labile linker based on cis-aconitic acid that takes advantage of the acidic environment of different intracellular compartments such as the endosomes encountered during receptor mediated endocytosis and the lysosomes. Shen and Ryser introduced this method for the preparation of conjugates of daunorubicin with macromolecular carriers (Biochem. Biophys. Res. Commun. 102:1048-1054 (1981)). Yang and Reisfeld used the same technique to conjugate daunorubicin to an anti-melanoma antibody (J. Natl. Canc. Inst. 80:1154-1159 (1988)). Recently, Dillman et al. also used an acid-labile linker in a similar fashion to prepare conjugates of daunorubicin with an anti-T cell antibody (Cancer Res. 48:6097-6102 (1988)).

An alternative approach, explored by Trouet et al. involved linking daunorubicin to an antibody via a peptide spacer arm (Proc. Natl. Acad. Sci. 79:626-629 (1982)). This was done under the premise that free drug could be released from such a conjugate by the action of lysosomal peptidases.

In vitro cytotoxicity tests, however, have revealed that antibody-drug conjugates rarely achieved the same cytotoxic potency as the free unconjugated drugs. This suggested that mechanisms by which drug molecules are released from the antibodies are very inefficient. In the area of immunotoxins, conjugates formed via disulfide bridges between monoclonal antibodies and catalytically active protein toxins were shown to be more cytotoxic than conjugates containing other linkers. See, Lambert et al. J. Biol. Chem. 260:12035-12041 (1985); Lambert et al. in Immunotoxins 175-209 (A. Frankel, ed. 1988); Ghetie et al. Cancer Res. 48:2610-2617 (1988). This was attributed to the high intracellular concentration of glutathione contributing to the efficient cleavage of the disulfide bond between an antibody molecule and a toxin. Despite this, there are only a few reported examples of the use of disulfide bridges for the preparation of conjugates between drugs and macromolecules. Shen et al. described the conversion of methotrexate into a mercaptoethylamide derivative followed by conjugation with poly-D-lysine via a disulfide bond (J. Biol. Chem. 260:10905-10908 (1985)). In addition, a report described the preparation of a conjugate of the trisulfide-containing toxic drug calicheamycin with an antibody (Menendez et al. Fourth International Conference on Monoclonal Antibody Immunoconjugates for Cancer, San Diego, Abstract 81 (1989)). Another report described the preparation of a conjugate of the trisulfide-containing toxic drug calicheamycin with an antibody (Hinman et al, 53 Cancer Res. 3336-3342 (1993)).

One reason for the lack of disulfide linked antibody-drug conjugates is the unavailability of cytotoxic drugs that bear a sulfur atom containing moiety that can be readily used to link the drug to an antibody via a disulfide bridge. Furthermore, chemical modification of existing drugs is difficult without diminishing their cytotoxic potential.

Another major drawback with existing antibody-drug conjugates is their inability to deliver a sufficient concentration of drug to the target site because of the limited number of targeted antigens and the relatively moderate cytotoxicity of cancerostatic drugs like methotrexate, daunorubicin and vincristine. In order to achieve significant cytotoxicity, linkage of a large number of drug molecules either directly to the antibody or through a polymeric carrier molecule becomes necessary. However such heavily modified antibodies often display impaired binding to the target antigen and fast in vivo clearance from the blood stream.

Maytansinoids are highly cytotoxic drugs. Maytansine was first isolated by Kupchan et al. from the east African shrub Maytenus serrata and shown to be 100 to 1000 fold more cytotoxic than conventional cancer chemotherapeutic agents like methotrexate, daunorubicin, and vincristine (U.S. Pat. No. 3,896,111). Subsequently, it was discovered that some microbes also produce maytansinoids, such as maytansinol and C-3 esters of maytansinol (U.S. Pat. No. 4,151,042). Synthetic C-3 esters of maytansinol and analogues of maytansinol have also been reported (Kupchan et al. J. Med. Chem. 21:31-37 (1978); Higashide et al. Nature 270:721-722 (1977); Kawai et al. Chem. Pharm. Bull. 32:3441-3451 (1984)). Examples of analogues of maytansinol from which C-3 esters have been prepared include maytansinol with modifications on the aromatic ring (e.g. dechloro) or at the C-9, C-14 (e.g. hydroxylated methyl group), C-15, C-18, C-20 and C-4,5.

The naturally occurring and synthetic C-3 esters can be classified into two groups:

(a) C-3 esters with simple carboxylic acids (U.S. Pat. Nos. 4,248,870; 4,265,814; 4,308,268; 4,308,269; 4,309,428; 4,317,821; 4,322,348; and 4,331,598), and

(b) C-3 esters with derivatives of N-methyl-L-alanine (U.S. Pat. Nos. 4,137,230; 4,260,608; 5,208,020; and Chem. Pharm. Bull. 12:3441 (1984)).

Esters of group (b) were found to be much more cytotoxic than esters of group (a).

Maytansine is a mitotic inhibitor. Treatment of L1210 cells in vivo with maytansine has been reported to result in 67% of the cells accumulating in mitosis. Untreated control cells were reported to demonstrate a mitotic index ranging from between 3.2 to 5.8% (Sieber et al. 43 Comparative Leukemia Research 1975, Bibl. Haemat. 495-500 (1976)). Experiments with sea urchin eggs and clam eggs have suggested that maytansine inhibits mitosis by interfering with the formation of microtubules through the inhibition of the polymerization of the microtubule protein, tubulin (Remillard et al. Science 189:1002-1005 (1975)).

In vitro, P388, L1210, and LY5178 murine leukemic cell suspensions have been found to be inhibited by maytansine at doses of 10⁻³ to 10⁻¹ .mu.g/.mu.l with the P388 line being the most sensitive. Maytansine has also been shown to be an active inhibitor of in vitro growth of human nasopharyngeal carcinoma cells, and the human acute lymphoblastic leukemia line CEM was reported inhibited by concentrations as low as 10⁻⁷ mg/ml (Wolpert-DeFillippes et al. Biochem. Pharmacol. 24:1735-1738 (1975)).

In vivo, maytansine has also been shown to be active. Tumor growth in the P388 lymphocytic leukemia system was shown to be inhibited over a 50- to 100-fold dosage range which suggested a high therapeutic index; also significant inhibitory activity could be demonstrated with the L1210 mouse leukemia system, the human Lewis lung carcinoma system and the human B-16 melanocarcinoma system (Kupchan, Ped. Proc. 33:2288-2295 (1974)).

Current methods of conjugation of maytansinoids with cell binding agents (such as antibodies) involve two reaction steps. A cell binding agent, for example an antibody, is first modified with a cross-linking reagent such as N-succinimidyl pyridyldithiopropionate (SPDP) to introduce dithiopyridyl groups into the antibody (Carlsson et al. Biochem. J. 173:723-737 (1978); U.S. Pat. No. 5,208,020). In a second step, a reactive maytansinoid having a thiol group, such as DM1 (formally termed N^{2′}-deacetyl-N^{2′}-(3-mercapto-1-oxopropyl)-maytansine, as the starting reagent, is added to the modified antibody, resulting in the displacement of the thiopyridyl groups in the modified antibodies, and the production of disulfide-linked cytotoxic maytansinoid/antibody conjugates (U.S. Pat. No. 5,208,020). Another methods using a succinimidyl-4-(N-maleimidomethyl)cyclohexane-1-carboxylate (SMCC) linker is described in the U.S. Ser. No. 11/927,217 patent application (Publication No. US 2008/0114153). A one-step process for conjugation of maytansinoids is described in U.S. Pat. No. 6,441,163. Maytansinoid-based immunotoxin technology is available from Immunogen Corporation (Cambridge, Mass.).

Another important toxin technology is based upon auristatin toxins. Auristatins are derived from Dolastatin 10 that was obtained from the Indian Ocean sea hare Dolabella, as a potent cell growth inhibitory substance. See U.S. Pat. Nos. 4,816,444 and 4,978,744. With respect to other Dolastatins, see also U.S. Pat. No. 4,414,205 (Dolastatin-1, 2, and 3), U.S. Pat. No. 5,076,973 (Dolastatin-3), U.S. Pat. No. 4,486,414 (Dolastatin-A and B), U.S. Pat. No. 4,986,988 (Dolastatin-13), U.S. Pat. No. 5,138,036 (Dolastatin-14), and U.S. Pat. No. 4,879,278 (dolastatin-15). Isolated and synthesized by Dr. Pettit and colleagues at the University of Arizona, a variety of auristatine derivatives have been tested and shown to be highly toxic to cells. See Pettit et al. Antineoplastic agents 337. Synthesis of dolastatin 10 structural modifications. Anticancer Drug Des. 10(7):529-44 (1995), Woyke et al. In vitro activities and postantifungal effects of the potent dolastatin 10 structural modification auristatin PHE. Antimicrobial Agents and Chemotherapy. 45:3580-3584 (2001), Pettit et al. Specific activities of dolastatin 10 and peptide derivatives against Cryptococcus neoformans. Antimicrobial Agents and Chemotherapy. 42:2961-2965 (1998), WoykeThree-dimensional visualization of microtubules during the Cryptococcus neoformans cell cycle and the effects of auristatin PHE on microtubule integrity and nuclear localization. Submitted, Antimicrobial Agents and Chemotherapy.

Recently, additional auristatin derivatives have been developed that appear quite effective when delivered as payloads on antibodies. For example monomethyl auristatin E (MMAE) has been shown as a potent toxin for tumor cells when conjugated to tumor specific antibodies. Doronina et al. Development of potent monoclonal antibody auristatin conjugates for cancer therapy. Nature Biotechnology. (2003) (available online), Francisco et al. cAC10-vcMMAE, an anti-CD30-monomethyl auristatin E conjugate with potent and selective antitumor activity. Blood. (2003) May 8 [Epub ahead of print]. Epub 2003 Apr. 24 (available online). In addition to the toxicity of the auristatin molecule, research has shown that peptide-linked conjugates are more stable, and, thus, more specific and less toxic to normal tissues than other linker technologies in buffers and plasma. Doronina et al. Development of potent monoclonal antibody auristatin conjugates for cancer therapy. Nature Biotechnology. (2003) (available online), Francisco et al. cAC10-vcMMAE, an anti-CD30-monomethyl auristatin E conjugate with potent and selective antitumor activity. Blood. (2003) May 8 [Epub ahead of print]. Epub 2003 Apr. 24 (available online). Such linkers are based on a branched peptide design and include, for example, mAb-valine-citrulline-MMAE and mAb-phenylalanine-lysine-MMAE conjugates. Doronina et al. Development of potent monoclonal antibody auristatin conjugates for cancer therapy. Nature Biotechnology. (2003) (available online), Francisco et al. cAC10-vcMMAE, an anti-CD30-monomethyl auristatin E conjugate with potent and selective antitumor activity. Blood. (2003) May 8 [Epub ahead of print]. Epub 2003 Apr. 24 (available online). Such designs and conjugation techniques are described, for example, by King et al. Monoclonal antibody conjugates of doxorubicin prepared with branched peptide linkers: inhibition of aggregation by methoxytriethyleneglycol chains. J Med Chem. 45(19):4336-43 (2002) and Dubowchik et al. Cathepsin B-sensitive dipeptide prodrugs. 2. Models of anticancer drugs paclitaxel (Taxol), mitomycin C and doxorubicin. Bioorg Med Chem Lett. 8(23):3347-52 (1998). Auristatin E-based immunotoxin technology based upon the foregoing is available from Seattle Genetics Corporation (Seattle, Wash.).

There are a large number of novel microtubule effecting compounds obtained from natural sources-extracts, and semisynthetic and synthetic analogs that appear to possess potential as toxins for the generation of immunoconjugates. (see the website at newmedinc “dot” com). Such molecules and examples of drug products utilizing them, include the following: Colchicine-site Binders (Curacin), Combretastatins (AVE806, Combretastatin A-4 prodrug (CA4P), Oxi-4503), Cryptophycins (LY355703), Discodermolide, Dolastatin and Analogs (Auristatin PHE, Dolastatin 10, ILX-651, Symplostatin 1, TZT-1027), Epothilones (BMS-247550, BMS-310705, EP0906, KOS-862, ZK-EPO), Eleutherobin, FR182877, Halichondrin B (E7389), Halimide (NPI-2352 and NPI-2358), Hemiasterlins (HTI-286), Laulimalide, Maytansinoids (“DM1”)(Bivatuzumab mertansine, Cantuzumab mertansine, huN901-DM1/BB-10901TAP, MLN591DM1, My9-6-DM1, Trastuzumab-DM1), PC-SPES, Peloruside A, Resveratrol, S-allylmercaptocysteine (SAMC), Spongistatins, Vitilevuamide, Molecular Motor-Kinesins (SB-715992), Designed Colchicine-Site Binders (A-289099, A-293620/A-318315, ABT-751/E7010, D-24851/D-64131, ZD6126), Other Novel Spindle Poisons (2-Methoxyestradiol (2-ME2), Bezimidazole Carbamates (ANG 600 series, Mebendazole), CP248/CP461, HMN-214, R440, SDX-103, T67/T607). Further, additional marine derived toxins are reviewed in Mayer, A. M. S. Marine Pharmacology in 1998: Antitumor and Cytotoxic Compounds. The Pharmacologist. 41(4):159-164 (1999).

### Therapeutic Administration and Formulations

A prolonged duration of action will allow for less frequent and more convenient dosing schedules by alternate parenteral routes such as intravenous, subcutaneous or intramuscular injection.

When used for in vivo administration, conjugated antibody formulations described herein, particularly Ab 131-DM1 conjugate, should be sterile. This is readily accomplished, for example, by filtration through sterile filtration membranes, prior to or following lyophilization and reconstitution. Conjugated antibodies ordinarily will be stored in lyophilized form or in solution. Therapeutic antibody compositions generally are placed into a container having a sterile access port, for example, an intravenous solution bag or vial having an adapter that allows retrieval of the formulation, such as a stopper pierceable by a hypodermic injection needle.

The route of antibody administration is in accord with known methods, e.g., injection or infusion by intravenous, intraperitoneal, intracerebral, intramuscular, intraocular, intraarterial, intrathecal, inhalation or intralesional routes, or by sustained release systems as noted below. Antibodies are preferably administered continuously by infusion or by bolus injection. In one embodiment Ab 131-DM1 conjugate is administered intravenously. In another embodiment Ab 131-DM1 conjugate is administered by injection to the tumor. In another embodiment Ab 131-DM1 conjugate is administered continuously directly to the tumor via an reservoir, depot formulation or implanted device.

In some embodiments of the invention particular dosing regimens for conjugated antibodies, such as Ab 131-DM1 conjugate are provided. The dosage of the antibody formulation for a given patient will be determined by the attending physician taking into consideration various factors known to modify the action of drugs including severity and type of disease, body weight, sex, diet, time and route of administration, other medications and other relevant clinical factors.

Typically, the clinician will administer antibody until a dosage is reached that achieves the desired effect. The progress of this therapy is easily monitored by the conventional assays and assays described herein including radiological imaging such as MRI.

Conjugated antibodies, as described herein, such as Ab 131-DM1 conjugate, can be prepared in a mixture with a pharmaceutically acceptable carrier. Therapeutic compositions can be administered intravenously or through the nose or lung, preferably as a liquid or powder aerosol (lyophilized). Composition can also be administered parentally or subcutaneously as desired. When administered systemically, therapeutic compositions should be sterile, pyrogen-free and in a parenterally acceptable solution having due regard for pH, such as a pH of 4 to about 6, isotonicity, and stability. These conditions are known to those skilled in the art.

Briefly, dosage formulations of the compounds are prepared for storage or administration by mixing the conjugated antibody, such as the Ab 131-DM1 conjugate, having the desired degree of purity with physiologically acceptable carriers, excipients, or stabilizers. Such materials are non-toxic to the recipients at the dosages and concentrations employed, and include buffers such as borate, succinate bicarbonate, sodium phosphate (“NaOAC”), Tris-HCl, Tris buffer, citrates, phosphate buffer, phosphate-buffered saline (i.e., PBS buffer), acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, sucrose, mannose, or dextrins; chelating agents such as EDTA; sugar alcohols such as mannitol or sorbitol; counterions such as sodium and/or nonionic surfactants such as TWEEN including polysorbate 20 or 80, PLURONICS, triton, tromethamine, lecithin, cholesterol, tyloxapal or polyethyleneglycol. Such dosage formulations can comprise a concentration of the compound, such as Ab 131-DM1, ranging from 1 mg/ml to 160 mg/ml.

Sterile compositions for injection can be formulated according to conventional pharmaceutical practice as described in Remington's Pharmaceutical Sciences (18^{th} ed, Mack Publishing Company, Easton, Pa., 1990). For example, dissolution or suspension of the active compound in a vehicle such as water or naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

Suitable examples of sustained-release preparations include semipermeable matrices of solid hydrophobic polymers containing the conjugated antibody, which matrices are in the form of shaped articles, films or microcapsules. Examples of sustained-release matrices include polyesters, hydrogels (e.g., poly(2-hydroxyethyl-methacrylate) as described by Langer et al., J. Biomed Mater. Res., (1981) 15:167-277 and Langer, Chem. Tech., (1982) 12:98-105, or poly(vinylalcohol)), polylactides (U.S. Pat. No. 3,773,919, EP 58,481), copolymers of L-glutamic acid and gamma ethyl-L-glutamate (Sidman et al., Biopolymers, (1983) 22:547-556), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through disulfide interchange, stabilization may be achieved by modifying sulfhydryl residues, lyophilizing from acidic solutions, controlling moisture content, using appropriate additives, and developing specific polymer matrix compositions.

Sustained-released compositions also include preparations of crystals of the antibody suspended in suitable formulations capable of maintaining crystals in suspension. These preparations when injected subcutaneously or intraperitoneally can produce a sustain release effect. Other compositions also include liposomally entrapped antibodies. Liposomes containing such antibodies are prepared by methods known per se: U.S. Pat. No. DE 3,218,121; Epstein et al., Proc. Natl. Acad. Sci. USA, (1985) 82:3688-3692; Hwang et al., Proc. Natl. Acad. Sci. USA, (1980) 77:4030-4034; EP 52,322; EP 36,676; EP 88,046; EP 143,949; 142,641; Japanese patent application 83-118008; U.S. Pat. Nos. 4,485,045 and 4,544,545; and EP 102,324.

It will be appreciated that administration of therapeutic entities in accordance with the compositions and methods herein will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (18^{th} ed, Mack Publishing Company, Easton, Pa. (1990)), particularly Chapter 87 by Block, Lawrence, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol. Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci 0.89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The therapeutic entities of the invention, such as Ab 131-DM1 can also be used in combination therapy. Combination therapy encompasses administration of the therapeutic molecule of the invention, such as Ab 131-DM1, to the mammal, preferably a human, prior to, in combination with or after treating the mammal by applying surgery, applying radiationtherapy, applying whole brain radiation therapy in the primary setting, applying focal radiation therapy in the recurrent setting, administering temozolomide in the primary and recurrent setting, administering bevacizumab, administering irinotecan, administering PCV, procabazine, lomustine [CCNU], vincristine, implanting a Gliadel wafer (polifeprosan impregnated with BCNU), administering a tyrosine kinase inhibitor, administering a radio-sensitizing agent, administering a vaccine based therapy, administering an antibody drug conjugate or administering a BiTE in the primary or recurrent settings or administering a targeted drug to the mammal.

In some embodiments the therapeutic molecule administered to the mammal in combination with the anti-EGFRvIII drug conjugate of the invention such as Ab 131-DM1, is another an anti-EGFRvIII therapeutic molecule such as an anti-EGFRvIII vaccine such as Rindopepimut, an anti-EGFRvIII antibody, another anti-EGFRvIII antibody drug conjugate, or an anti-EGFRvIII Bi-specific T-cell engager.

In some embodiments the therapeutic molecule administered to the mammal in combination with the anti-EGFRvIII drug conjugate of the invention such as Ab 131-DM1, is an anti-EGFR therapeutic molecule such panitumumab, cetuximab, other anti-EGFR antibody, anti-EGFR vaccine, anti-EGFR antibody drug conjugate or anti-EGFR Bi-specific T-cell engager.

In some embodiments the therapeutic molecule administered to the mammal in combination with the anti-EGFRvIII drug conjugate of the invention such as Ab 131-DM1, is an anti-Interleukin-6 therapeutic molecule such as an anti-Interleukin-6 antibody such as siltuximab, anti-Interleukin-6 receptor antibody such as tocilizumab, an anti-Interleukin-6 or anti-Interleukin-6 receptor antibody drug conjugate, or an anti-Interleukin-6 or anti-Interleukin-6 receptor Bi-specific T-cell engager.

In some embodiments the therapeutic molecule administered to the mammal in combination with the anti-EGFRvIII drug conjugate of the invention such as Ab 131-DM1, is an anti-Interleukin-8 therapeutic molecule such as an anti-Interleukin-8 antibody, an anti-Interleukin-8 receptor antibody such as, an anti-Interleukin-8 or anti-Interleukin-8 receptor antibody drug conjugate, or an anti-Interleukin-8 or anti-Interleukin-8 receptor Bi-specific T-cell engager.

In some embodiments the anti-EGFRvIII antibody drug conjugate of the invention, such as Ab 131-DM1 is administered prior to, in combination with or after administration of or more anti-EGFRvIII, anti-EGFR, anti-Interleukin-6, anti-Interleukin 6 receptor, anti-Interleukin-8 or anti-interleukn-8 receptor therapeutic molecules are administered to the mammal.

Such combination therapy is useful in treating a mammal having a tumor expressing EGFRvIII, a lung carcinoma, breast carcinoma, colon carcinoma, gastric carcinoma, renal carcinoma, head & neck carcinoma, prostate carcinoma, ovarian carcinoma, glioblastoma, an anaplastic astrocytoma, astrocytoma or a tumor comprising a glial component, particularly glioblastoma, anaplastic astrocytoma, astrocytoma, recurrent glioblastoma, recurrent anaplastic astrocytoma, oligodenroglioma, oligoastrocytoma, gliosarcoma, mixed glioma, pilocytic astrocytoma, pleomorphic xanthoastrocytoma, sub ependymal giant cell astrocytoma, astroblastoma, spongioblastoma, gliomatosis cerebri, or neuronal-glial tumors including gangliglioma, or anaplastic ganglioglioma.

### Preparation of Conjugated Antibodies

Conjugated Antibodies, as described herein, were prepared through the utilization of the XenoMouse® technology, as described below and as described in U.S. Pat. No. 7,628,986 which herein incorporated by reference in its entirety. Such mice, then, are capable of producing human immunoglobulin molecules and antibodies and are deficient in the production of murine immunoglobulin molecules and antibodies. Technologies utilized for achieving the same are disclosed in the patents, applications, and references disclosed herein. In particular, however, a one embodiment of transgenic production of mice and antibodies there from is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics: 146-156 (1997), the disclosure of which is hereby incorporated by reference.

Through use of such technology, fully human monoclonal antibodies to a variety of antigens can be produced. In one embodiment, XenoMouse® lines of mice are immunized with an antigen of interest (e.g. EGFRvIII), lymphatic cells are recovered (such as B-cells) from the mice that expressed antibodies, and such cells are fused with a myeloid-type cell line to prepare immortal hybridoma cell lines, and such hybridoma cell lines are screened and selected to identify hybridoma cell lines that produce antibodies specific to the antigen of interest. Provided herein are methods for the production of multiple hybridoma cell lines that produce antibodies specific to EGFRvIII. Further, provided herein are characterization of the antibodies produced by such cell lines, including nucleotide and amino acid sequences of the heavy and light chains of such antibodies.

Alternatively, instead of being fused to myeloma cells to generate hybridomas, the antibody produced by recovered cells, isolated from immunized XenoMouse® lines of mice, are screened further for reactivity against the initial antigen, preferably EGFRvIII protein. Such screening includes ELISA with EGFRvIII protein, in vitro binding to NR6 M cells stably expressing full length EGFRvIII and internalization of EGFRvIII receptor by the antibodies in NR6 M cells. Single B cells secreting antibodies of interest are then isolated using a EGFRvIII-specific hemolytic plaque assay (Babcook et al., Proc. Natl. Acad. Sci. USA, i93:7843-7848 (1996)). Cells targeted for lysis are preferably sheep red blood cells (SRBCs) coated with the EGFRvIII antigen. In the presence of a B cell culture secreting the immunoglobulin of interest and complement, the formation of a plaque indicates specific EGFRvIII-mediated lysis of the target cells. The single antigen-specific plasma cell in the center of the plaque can be isolated and the genetic information that encodes the specificity of the antibody is isolated from the single plasma cell. Using reverse-transcriptase PCR, the DNA encoding the variable region of the antibody secreted can be cloned. Such cloned DNA can then be further inserted into a suitable expression vector, preferably a vector cassette such as a pcDNA, more preferably such a pcDNA vector containing the constant domains of immuoglobulin heavy and light chain. The generated vector can then be transfected into host cells, preferably CHO cells, and cultured in conventional nutrient media modified as appropriate for inducing promoters, selecting transformants, or amplifying the genes encoding the desired sequences. Herein, we describe the isolation of multiple single plasma cells that produce antibodies specific to EGFRvIII. Further, the genetic material that encodes the specificity of the anti-EGFRvIII antibody is isolated, introduced into a suitable expression vector that is then transfected into host cells.

B cells from XenoMouse mice may be also be used as a source of genetic material from which antibody display libraries may be generated. Such libraries may be made in bacteriophage, yeast or in vitro via ribosome display using ordinary skills in the art. Hyperimmunized XenoMouse mice may be a rich source from which high-affinity, antigen-reactive antibodies may be isolated. Accordingly, XenoMouse mice hyperimmunized against EGFRvIII may be used to generate antibody display libraries from which high-affinity antibodies against EGFRvIII may be isolated. Such libraries could be screened against the pep3 oligopeptide and the resultingly derived antibodies screening against cells expressing EGFRvIII to confirm specificity for the natively display antigen. Full IgG antibody may then be expressed using recombinant DNA technology. See e.g., WO 99/53049.

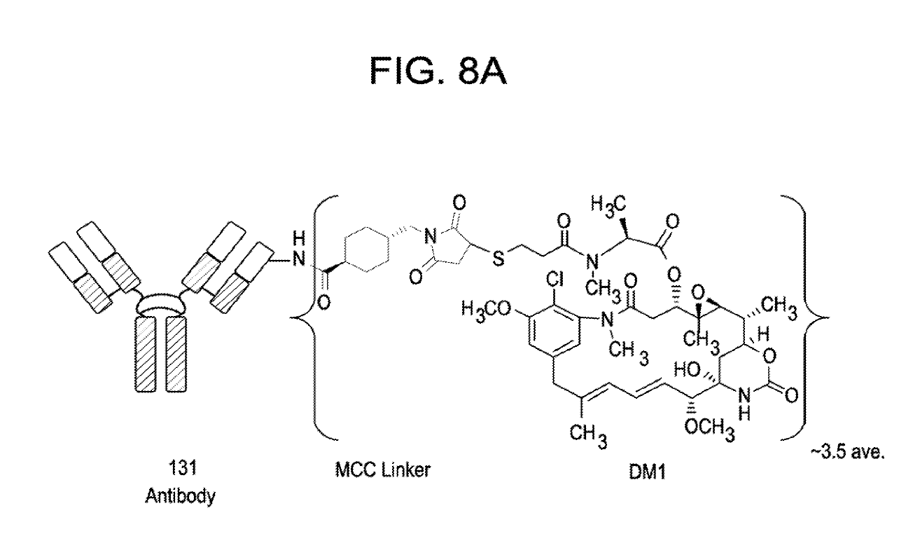

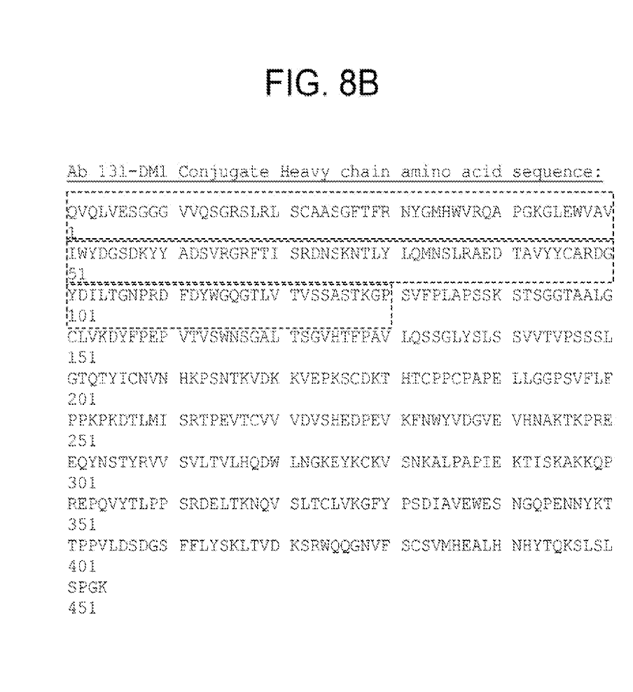

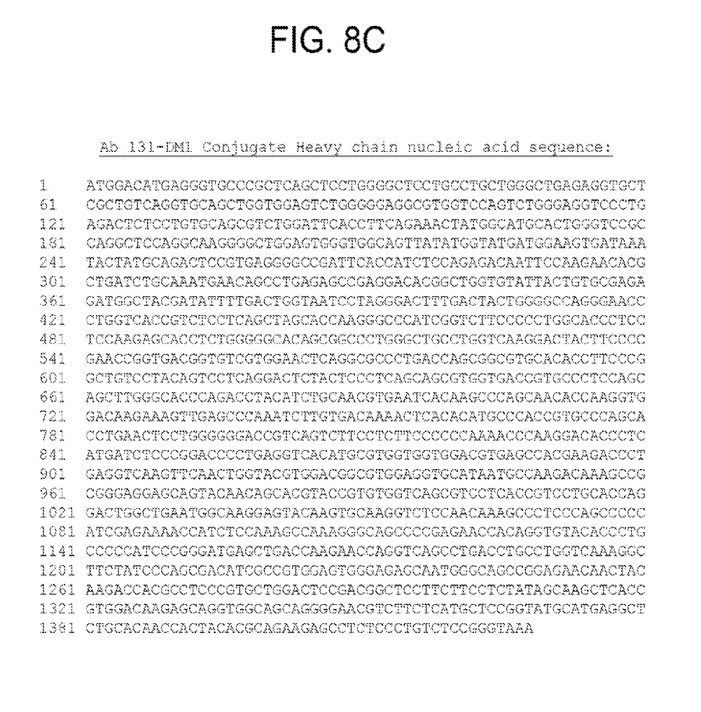

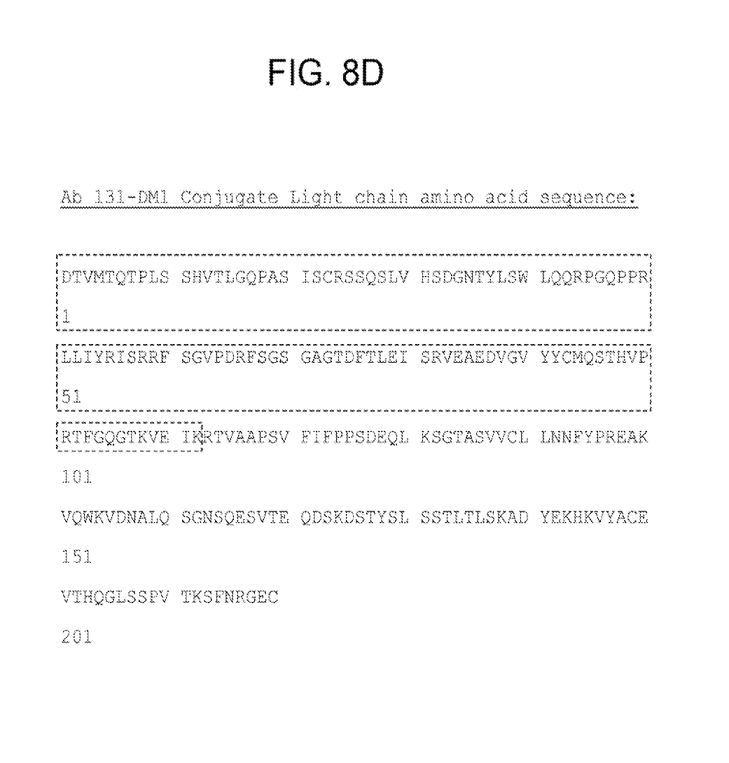

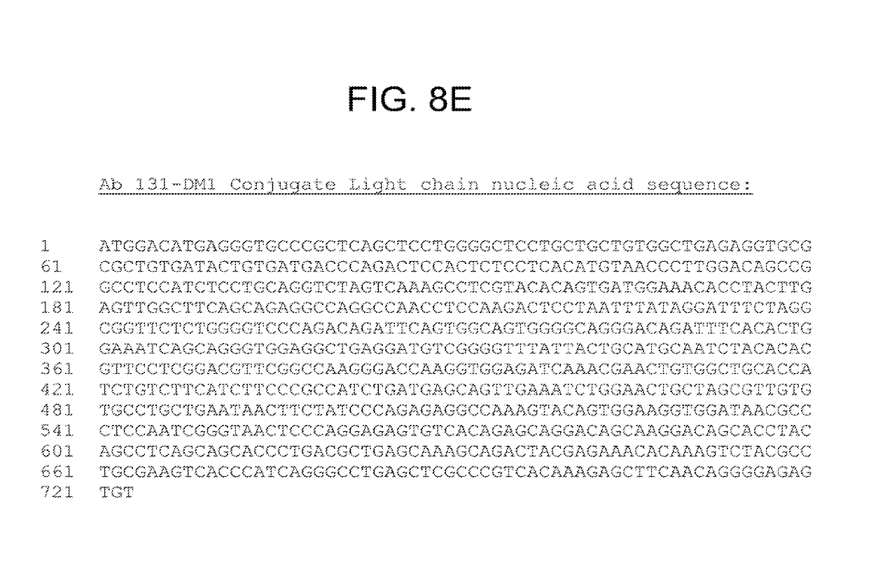

For example, the antibodies of the conjugated antibodies of the invention may be produced from transfected cells. Cells (293 cells for transient expression and CHO cells for stable expression) may be transfected with plasmids that encode the heavy and light chains of the Ab 131-DM1 conjugate depicted in FIG. 8 of this application. Conditioned media from the transfected cells may be recovered by removing cells and cell debris. Clarified conditioned media may be loaded onto a Protein A-Sepharose column. Optionally, the media can first be concentrated and then loaded onto a Protein A Sepharose column. Non-specific bindings may be removed by extensive PBS wash. Bound antibody proteins on the Protein A column may be recovered by standard acidic antibody elution from Protein A columns (50 mM Citrate, pH 3.0). Aggregated antibody proteins in the Protein A Sepharose pool may be removed by size exclusion chromatography or binding ion exchange chromatography on cation exchanger resin such as SP-Sepharose resin. Antibodies may be eluted with excess column volumes of buffer.

In general, antibodies produced by the above-mentioned cell lines possessed fully human IgG1 or IgG2 heavy chains with human kappa light chains. In one embodiment, the antibodies possessed high affinities, typically possessing Kd's of from about 10⁻⁹ through about 10⁻¹³ M, when measured by either solid phase and solution phase. In other embodiments the antibodies possessed lower affinities, from about 10⁻⁶ through about 10⁻⁸ M.

As appreciated by one of skill in the art, antibodies in accordance with the present embodiments can be expressed in cell lines other than hybridoma cell lines. Sequences encoding particular antibodies can be used for transformation of a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introduction of heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels and produce antibodies with constitutive EGFRvIII binding properties such as Ab 131-DM1 conjugate.

### EXAMPLES

The following examples, including the experiments conducted and the results achieved are provided for illustrative purposes only and are not to be construed as limiting upon the present invention.

The strategy for generating EGFRvIII-specific antibodies initially involved immunization of XenoMouse mice with combinations of antigens (peptide, various soluble proteins, antigen-expressing cells) followed by isolation of antibody producing cells, either as through fusions to produce hybridomas or isolation of B cell cells through the XenoMax™/SLAM™ technology. Antibody producing cells were subjected to a primary screen for specificity by ELISA and a secondary screen for cell surface binding by FMAT and/or FACS. Internalization assays were then conducted to identify antibodies that would be useful for drug delivery. Affinities of the antibodies were measured. Certain antibodies were selected for epitope mapping. In addition, certain antibodies were selected for in vitro and in vivo tests to analyze the efficacy of such antibodies for treatment of cancers.

### Example 1

### Antigen Preparation

### A. EGFRvIII PEP3-KLH Antigen Preparation

In connection with Example 2, the 14-mer human EGFRvIII PEP3 (L E E K K G N Y V V T DHC (SEQ ID NO: 56)) peptide was custom synthesized by R&D Systems. The PEP3 peptide was then conjugated to keyhole limpet hemocyanin (KLH), as follows: EGFRvIII PEP3 (200 mcg) (R&D) was mixed with 50 mcg of keyhole limpet hemocyanin (KLH; Pierce, Rockford, Ill.) to a final volume of 165 mcl using distilled water. 250 mcl of conjugation buffer (0.1M MES, 0.9M NaCl, pH 4.7) was added and EGFRvIII PEP3 and KLH were crosslinked by the addition of 25 mcl of 10 mg/ml stock solution of 1-ethyl-3-[3-dimethylaminopropyl]carbodiimide hydrochloride (EDC, Pierce, Rockford, Ill.). Conjugate was incubated for 2 hours at room temperature and the unreacted EDC was removed by centrifugation through a 1 kDa filter (Centrifugal filter; Millipore, Bedford, Mass.) using PBS pH 7.4.

In connection with Example 3, the 14-mer human EGFRvIII PEP3 (L E E K K G N Y V V T D H C (SEQ ID NO: 56)) peptide was custom synthesized. The PEP3 peptide was then conjugated to KLH, as follows: human EGFRvIII PEP3 (200 mcg) was mixed with 50 mcg of keyhole limpet hemocyanin (KLH; Pierce, Rockford, Ill.) to a final volume of 165 mcl using distilled water. 250 mcl of conjugation buffer (0.1M MES, 0.9M NaCl, pH 4.7) was added and EGFRvIII PEP3 and KLH were crosslinked by the addition of 25 mcl of 10 mg/ml stock solution of 1-ethyl-3-[3-dimethylaminopropyl]carbodiimide hydrochloride (EDC, Pierce, Rockford, Ill.). Conjugate was incubated for 2 hours at room temperature and the unreacted EDC was removed by centrifugation through a 1 kDa filter (Centrifugal filter; Millipore, Bedford, Mass.) using PBS pH 7.4.

### B. B300.19/EGFRvIII Transfectants

In order to prepare the B300.19/EGFRvIII transfectants, wild type EGFR was initially cloned from A431 cells and EGFR gene was modified to code for EGFRvIII to delete the codons encoding residues 6-273, with a codon encoding a Glycine residue created at the junction of the deletion. The deletion occurs within the codons surrounding the deletion GTT (Valine) and CGT (Arginine), such that the resulting codon after the deletion is GGT (Glycine). (Wikstrand et al. J Neurovirol. 4(2):148-58 (1998))

1. Cloning of Wild Type EGFR Construct:

PolyA+mRNA was extracted from A431 (ATCC) cells using Micro-fast RNA kit (Invitrogen, Burlington, ON). Total cDNA was synthesized from polyA+mRNA with random pdN6 primers and M-MuLV reverse transcriptase (NEB, New England Biolabs, Beverly, Mass.). A 2.3kb PCR product was amplified from A431 cDNA with the following primers:

sense

(SEQ ID NO: 62)

5′-GGATCTCGAGCCAGACCGGAACGACAGGCCACCTC-3′;
anti-sense

(SEQ ID NO: 63)

5′-CGGATCTCGAGCCGGAGCCCAGCACTTTGATCTT-3′

using Pfu DNA polymerase.

The PCR product was digested with XhoI, gel purified and ligated into plasmid pWBFNP (see International Patent Application No. WO 99/45031, the disclosure of which is hereby incorporated by reference) linearized with XhoI to yield plasmid Wt-EGFR/pWBFNP.

2. Generation of EGFRvIII Construct:

PCR products amplified from plasmid Wt-EGFR/pWBFNP template with primer pairs C13659/C29538 and C29539/C14288 (BioSource International), in which the C29538 and C29539 were phosphorylated with T4 Polynucleotide kinase (NEB, New England Biolabs, Beverly, Mass.):

C13659:

(SEQ ID NO: 64)

5′-CGGATGAATTCCCAGACCGGACGACAGGCCAC
CTC-3′(Sense);
C29538:

(SEQ ID NO: 65)

5′-CTTTCTTTTCCTCCAGAGCC-3′(Anti-Sense);
C29539:

(SEQ ID NO: 66)

5′-GTAATTATGTGGTGACAGATC-3′(Sense);
C14288:

(SEQ ID NO: 67)

5′-CGGATCTCGAGCTCAAGAGAGCTTGGTTGGGA
GCT-3′(Anti-Sense).

were ligated to introduce a deletion in the sequence encoding amino acids 6 through 273 of the EGFR extracellular domain and subcloned into expression vector pWBDHFR2 (see International Patent Application No. WO 99/45031, the disclosure of which is hereby incorporated by reference).

A 232 bp fragment representing the 5′end of the deletion was generated with primer pair C13659/C29538 from Wt-EGFR/pWBFNP template amplified with Pfu polymerase (NEB, New England Biolabs, Beverly, Mass.). The PCR product was digested with EcoR1 (NEB, New England Biolabs, Beverly, Mass.) and gel purified. A 1273 bp fragment representing the 3′end of the deletion was generated with primer pair C29539/C14288 from Wt-EGFR/pWBFNP and the template amplified with Pfu polymerase. The PCR product was digested with Xho1 (NEB, New England Biolabs, Beverly, Mass.) and gel purified. Fragments were ligated into EcoR1/Xho1 digested pWBDHFR2 with T4 DNA ligase (NEB, New England Biolabs, Beverly, Mass.) to yield construct EGFRvIII/pWBDHFR.

The intracellular domain of EGFR was introduced into the resulting construct as follows: A 1566 bp DraIII/XhoI fragment was isolated from plasmid Wt-EGFR/pWBFNP and ligated into DraIII/XhoI digested EGFRvIII/pWBDHFR to yield EGFRvIII-FL/pWBDHFR.

3. Transfection of B300.19 Cells with EGFRvIII-FL/pWBDHFR:

B300.19 cells (8×10⁶) were used per transfection in 700 μl DMEM/HI medium. 20 μg EGFRvIII-FL/pWBDHFR and 2 μg CMV-Puro plasmid DNA were added. Cells were electroporated at 300 volts/960 uF with Bio-Rad Gene Pulser. Following electroporation, cells were cooled on ice for 10 minutes and, thereafter, 10 ml non-selection medium (DMEM/HI Glucose, 10% FBS, 50 μM BME, 2 mM L-Glutamine, 100 units Penicillin-G/ml, 100 units MCG Streptomycin/ml) was added. Cells were incubated for 48 hrs at 37° C. 7.5% CO₂.

Following incubation, cells were split into selection medium (DMEM/HI Glucose, 10% FBS, 2 mM L-Glutamine, 50 μM BME, 100 units Penicillin-G/ml, 100 units MCG Streptomycin/ml, 2 ug/ml puromycin) at 2×10⁴, 0.4×10⁴′ and 0.08×10⁴ cells/well in 96 well plate and were selected in selection medium for 14 days to generate stable clones. Puro resistant clones were stained with E752 mAb (an anti-EGFR antibody, described in Yang et al., Crit Rev Oncol Hematol., 38(1):17-23 (2001)) and goat anti-human IgG PE then analyzed on FACS Vantage (Becton Dickinson).

### C. Construction of EGFRvIII-RbFc Expression Constructs.

In order to generate the EGFRvIII rabbit Fc fusion, protein, we first constructed a vector containing DNA encoding rabbit Fc. This was ligated with DNA encoding EGFRvIII. This approach is described in more detail below:

1. Construction of RbFc/pcDNA3.1 Hygro:

Primers 1322/867 (below) were used to amplify a 721 bp fragment encoding the Hinge-CH2-CH3 domain of rabbit IgG.

#1322 (sense):

(SEQ ID NO: 68)

5′-GGTGGCGGTACCTGGACAAGACCGTTGCG-3′
#867 (antisense):

(SEQ ID NO: 69)

5′-ATAAGAATGCGGCCGCTCATTTACCCGGAGAGCGGGA-3′

The resulting PCR product was digested with KpnI and NotI, gel purified and ligated into KpnI/NotI digested pcDNA3.1(+)/Hygro (Invitrogen, Burlington, ON) to yield plasmid RbFc/pcDNA3.1 Hygro.

2. Construction of EGFRvIII-RbFc/pCEP4:

Primers 1290/1293 (below) were used to amplify an 1165 bp product from EGFRvIII-FL/pWBDHFR plasmid template with Pfu polymerase

#1290 (sense):

(SEQ ID NO: 70)

5′-CTACTAGCTAGCCACCATGCGACCCTCCGGGA-3′
#1293 (anti-sense):

(SEQ ID NO: 71)

5′-CGGGGTACCCGGCGATGGACGGGATC-3′

The PCR product was digested with NheI and KpnI, gel purified and ligated into NheI/KpnI digested RbFc/pcDNA3.1 Hygro to yield plasmid EGFRvIII-RbFc/pcDNA3.1 Hygro.

A 2170 bp SnaBI/XhoI fragment was isolated from EGFRvIII-RbFc/pcDNA3.1Hygro and subcloned into SnaBI/XhoI digested pCEP4 (Invitrogen, Burlington, ON) to yield plasmid EGFRvIII-RbFc/pCEP4.

3. Generation of 293F EGFRvIII-RbFc Stable Cell Lines:

Plasmid EGFRvIII-RbFc/pCEP4 was introduced into 293F cells (Gibco, Grand Island, N.Y.) by Calcium Phosphate transfection, as follows: one day prior to transfection, 1×10⁶293 F cells were plated on a gelatin coated 100 mm tissue culture petridish and incubated at 5% CO2, 37° C. Cells were fed with 10 ml of fresh non-selective media (DMEM/F12, 10% FBS, 2 mM L-Glutamine, 100 U/ml Penicillin G, 100 U/ml MCG Streptomycin) 2-3 hours before transfection. Transfection reagents were prepared in a microfuge tube, as follow: 10 μg of DNA (EGFRvIII-RbFc/pCEP4) was mixed with 62 μl of 2M Calcium Phosphate and deionized water to make the final volume 500 μl. In another tube pipette 500 μl of 2× HBS is drawn and used to transfer the transfection reagents.

The solution in the tube pipette was added to the cells drop by drop, while maintaining proper pH by leaving cells in a 5% CO2 incubator until transfection was performed. 15-20 hours after transfection, cells were washed with PBS and feed with 10 ml of fresh 293F non-selective media. Expressing cells were harvested with trypsin 48-72 post-transfection and cells were plated at 0.08×10⁴ cells/well in a 96 well plate in 293F selective media (DMEM/F12, 10% FBS, 2 mM L-Glutamine, 100 U/ml Penicillin G, 100 U/ml MCG Streptomycin, 250 ug/ml Hygromycin) for 14 days.

Hygromycin resistant clones were screened by ELISA using anti-EGFR antibody E763 (U.S. Pat. No. 6,235,883) as the capture antibody at 1 ug/ml and detecting with a goat anti-rabbit IgG HRPO (CalTag) at 1:100 dilution.

### D. Conjugation of EGFRvIII PEP3 to OVA Via Maleimide Conjugation

The EGFRvIIIpeptide-OVA used for titration of antibodies (Example 3) was produced as follows:

207 μg of EGFRvIII PEP3 was reduced using pre-weighed DTT from Pierce (#20291). One vial of 7.7 mg of pre-weighed DTT was dissolved using 100 μL of deionized water. The DTT stock was added to the EGFRvIII PEP3. The volume of the reaction was brought to 600 μL using PBS pH 7.4. The reaction was rotated for 30 minutes at room temperature.

A G10 column was prepared by weighing out 5 grams of G10 sephadex beads and adding 40 mL of PBS, mixing and leaving at room temperature for 10 minutes, and then centrifuging the beads at 1000 rpm for 10 minutes. The supernatant was removed and an additional 20 mL of PBS was added. The beads were centrifuged at 1000 rpm for 10 minutes. The supernatant was removed and enough PBS added to make a 50% slurry of G10 sephadex beads. 5 mL of the 50% slurry mixture was added to a 5 mL spin column and the column was placed in a 14 mL polypropylene tube. The column was centrifuged at 1000 rpm for 3 minutes. Another 3 mL of PBS was added and the column was centrifuged again at 1000 rpm for 3 minutes. The polypropylene tube was replaced with a new tube and the columns were now ready to use.

DTT was removed from the reduced peptide. After the 30 minute reaction time for reducing the peptide, 300 μL of the reduced peptide was added per column. The column was centrifuged at 1000 rpm for 3 minutes. An additional 250 μL of PBS was added to each column and centrifuged again at 1000 rpm for 3 minutes. The reduced peptide was collected in the 14 mL polypropylene tube.

The reduced peptide was conjugated to maleimide activated OVA and collected in an eppendorf tube. 2 mg of the maleimide activated OVA was dissolved (Pierce: 77126, Rockford Ill.) with maleimide conjugation buffer to make a 10 mg/mL stock. 414 μg of the maleimide activated OVA was added to the reduced peptide in the eppendorf tube. 500 μL of the maleimide conjugation buffer was added to the reaction. The reaction was allowed to incubate for 2 hours at room temperature and then 2 mg of cysteine was added to quench any active maleimide groups that might have been present. The cysteine was allowed to react for 30 additional minutes at room temperature. The conjugate was then washed with a 10K centrifugal column 3 times using 1× PBS pH 7.4. This removed any free peptide that did not conjugate to the OVA and free cysteine. The conjugate was removed from the centrifugal column using gel loading tips and transferred to an eppendorf tube. Finally, the conjugate was brought to the desired concentration using 1× PBS pH 7.4. The conjugate produced had a molar ratio of 14.5:1 (peptide:OVA)

### Example 2

### Production of Anti-EGFRvIII Antibodies Through Hybridoma Generation

Eight XenoMouse mice that produce antibodies with a gamma-1 constant region (XenoMouse G1 mice) were immunized on day 0 and boosted on days 11, 21, 32, 44 and 54 for this protocol and fusions were performed on day 58. All immunizations were conducted via subcutaneous administration at the base of tail plus intraperitoneal administration for all injections. The day 0 immunization was done with 1.5×10⁷ B300.19/EGFRvIII transfected cells (Example 1A) suspended in pyrogen free DPBS admixed 1:1 v/v with complete Freunds adjuvant (CFA) (Sigma, St. Louis, Mo.). Boosts on days 11, 21, and 32 were done with 1.5×10⁷ B300.19/EGFRvIII transfected cells in DPBS admixed 1:1 v/v with incomplete Freunds adjuvant (IFA) (Sigma, St. Louis, Mo.). The boosts on day 44 was done with 5 μg of the PEP3 (EGFRvIII peptide)—KLH conjugate (Example 1) in DPBS admixed 1:1 v/v with IFA and final boost, on day 54, was done with 5 ug PEP3 (EGFRvIII peptide)—KLH conjugate in DPBS without adjuvant.

On day 58, mice were euthanized, and then inguinal and Lumbar lymph nodes were recovered. Lymphocytes were released by mechanical disruption of the lymph nodes using a tissue grinder then depleted of T cells by CD90 negative selection. The fusion was performed by mixing washed enriched B cells and non-secretory myeloma P3X63Ag8.653 cells purchased from ATCC, cat. # CRL 1580 (Kearney et al, J. Immunol. 123:1548-1550 (1979)) at a ratio of 1:1. The cell mixture was gently pelleted by centrifugation at 800 g. After complete removal of the supernatant, the cells were treated with 2-4 mL of Pronase solution (CalBiochem, cat. #53702; 0.5 mg/ml in PBS) for no more than 2 minutes. Then, 3-5 ml of FBS was added to stop the enzyme activity and the suspension was adjusted to 40 ml total volume using electro cell fusion solution, ECFS (0.3M Sucrose, Sigma, Cat# S7903, 0.1 mM Magnesium Acetate, Sigma, Cat# M2545, 0.1 mM Calcium Acetate, Sigma, Cat# C4705 (St. Louis, Mo.)).

The supernatant was removed after centrifugation and the cells washed by resuspension in 40 ml ECFS. This wash step was repeated and the cells again were resuspended in ECFS to a concentration of 2×10⁶ cells/ml. Electro-cell fusion was performed using a fusion generator, model ECM2001, Genetronic, Inc., San Diego, Calif. The fusion chamber size used was 2.0 ml, and using the following instrument settings: Alignment condition: voltage: 50 v, time: 50 s, Membrane breaking at: voltage: 3000 v, time: 30 μs, Post-fusion holding time: 3 s. After fusion, the cells were re-suspended in DMEM (JRH Biosciences), 15% FCS (Hyclone), containing HAT, and supplemented with L-glutamine, pen/strep, OPI (oxaloacetate, pyruvate, bovine insulin) (all from Sigma, St. Louis, Mo.) and IL-6 (Boehringer Mannheim) for culture at 37° C. and 10% CO² in air.

Cells were plated in flat bottomed 96-well tissue culture plates at 4×10⁴ cells per well. Cultures were maintained in HAT (hypoxanthine, aminopterin and thymidine) supplemented media for 2 weeks before transfer to HT (hypoxanthine and thymidine) supplemented media. Hybridomas were selected for by survival in HAT medium and supernatants were screened for antigen reactivity by ELISA. The ELISA format entailed incubating supernatants on antigen coated plates (EGFRvIII peptide-OVA coated plates and wild type EGFr peptide-OVA coated plates as a counter screen) and detecting EGFRvIII-specific binding using horseradish peroxidase (HRP) labeled mouse anti-human IgG (see Table 2.1).

TABLE 2.1

| | |
|---|---|
| 1^{st} OD | 2nd OD |

| Plate.Well | Hybridoma | fusion plate | muEGFr | EGFr |
|---|---|---|---|---|
| 13.2 D10 | 13.1 | 4.034 | 2.653 | 0.051 |
| 13.3 C12 | 13.2 | 3.829 | 2.443 | 0.049 |
| 13.3 F11 | 13.3 | 3.874 | 1.081 | 0.049 |
| 13.6 B11 | 13.4 | 3.322 | 1.311 | 0.052 |

| | |
|---|---|
| OD #1 | OD #2 |

| Clones | Plate | cloning plate | muEGFr | EGFr |
|---|---|---|---|---|
| 13.1.1 | 0.5c/w D2 | 2.614 | 2.586 | 0.042 |
| 13.1.2 | 0.5c/w F5 | 2.248 | 1.272 | 0.041 |

As will be observed, at least four antigen specific hybridomas were detected: 13.1, 13.2, 13.3, and 13.4. These hybridomas that were positive in the ELISA assay EGFRvIII specificity were confirmed by FACS on stably transfected 300.19 cells expressing EGFRvIII versus 300.19 untransfected parental cells.

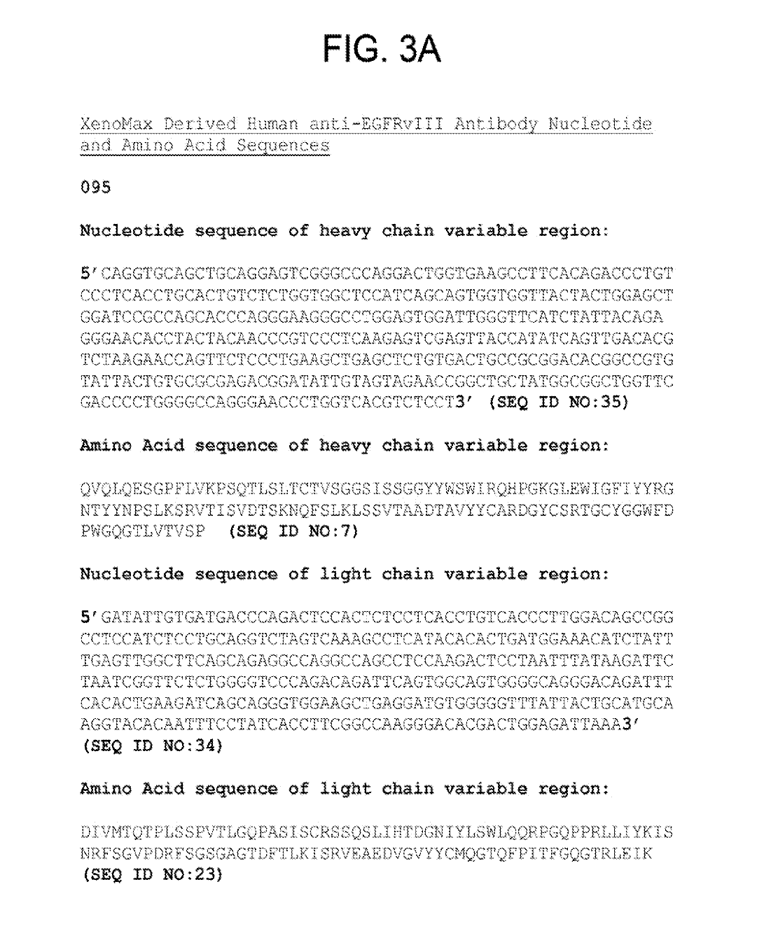

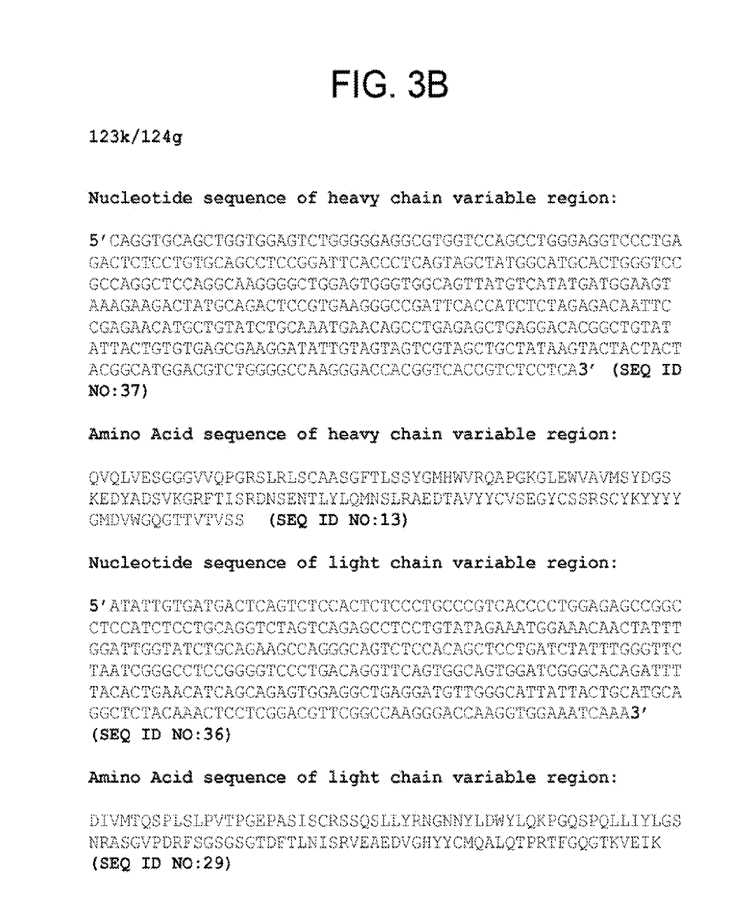

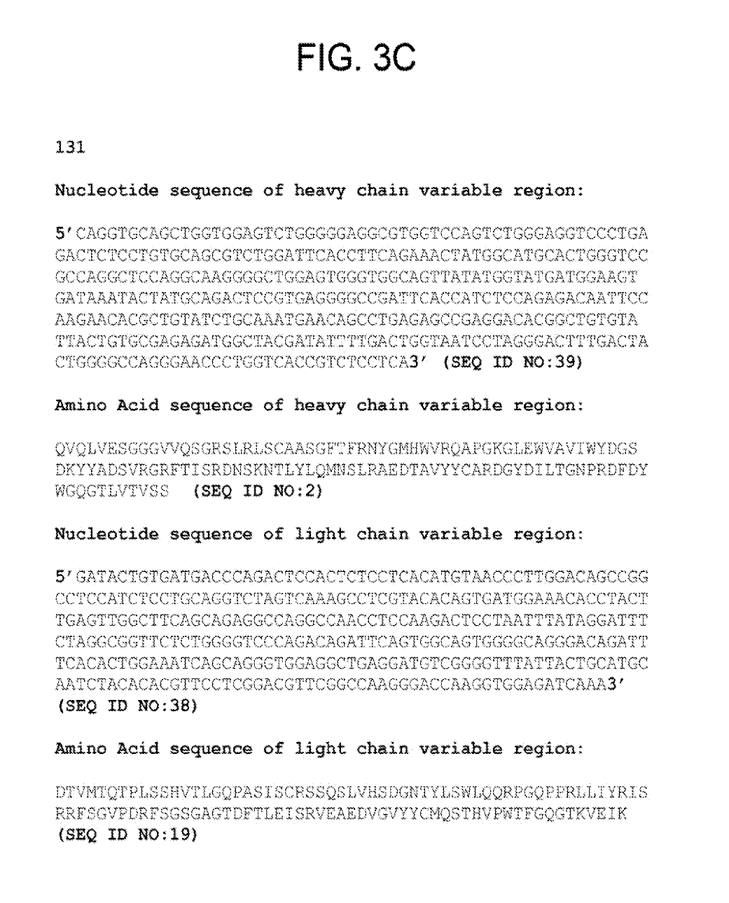

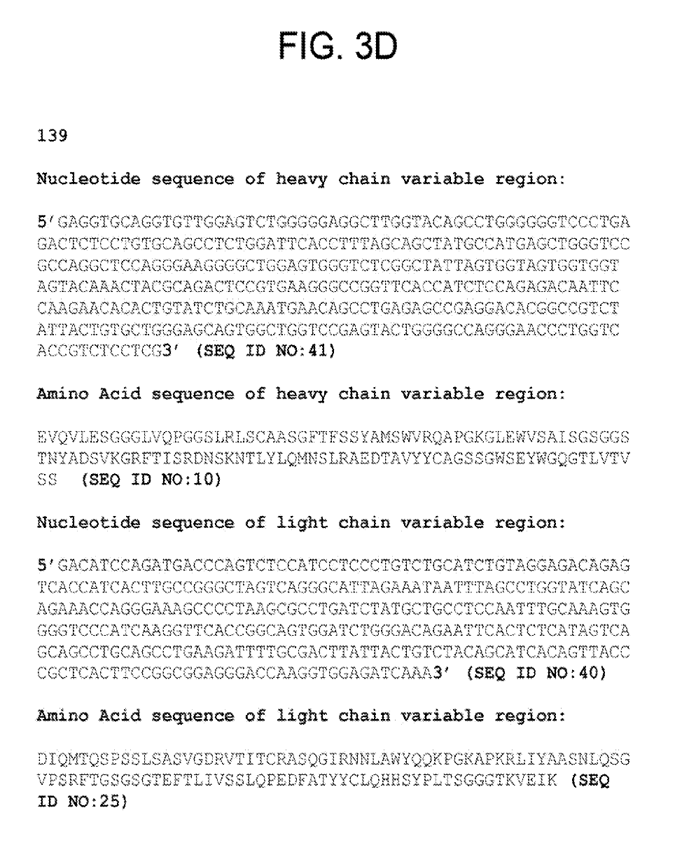

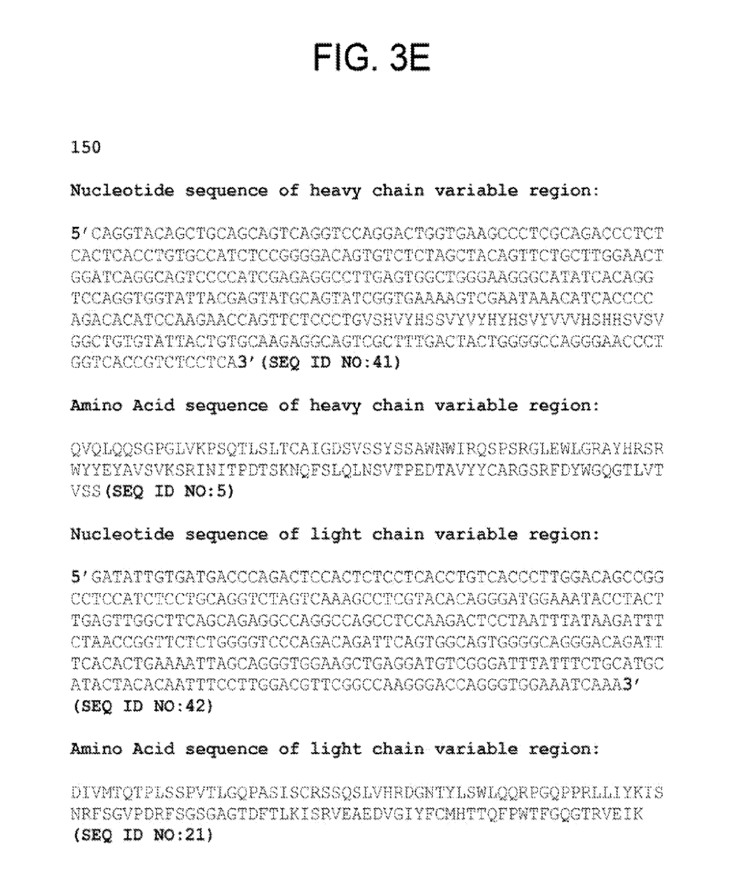

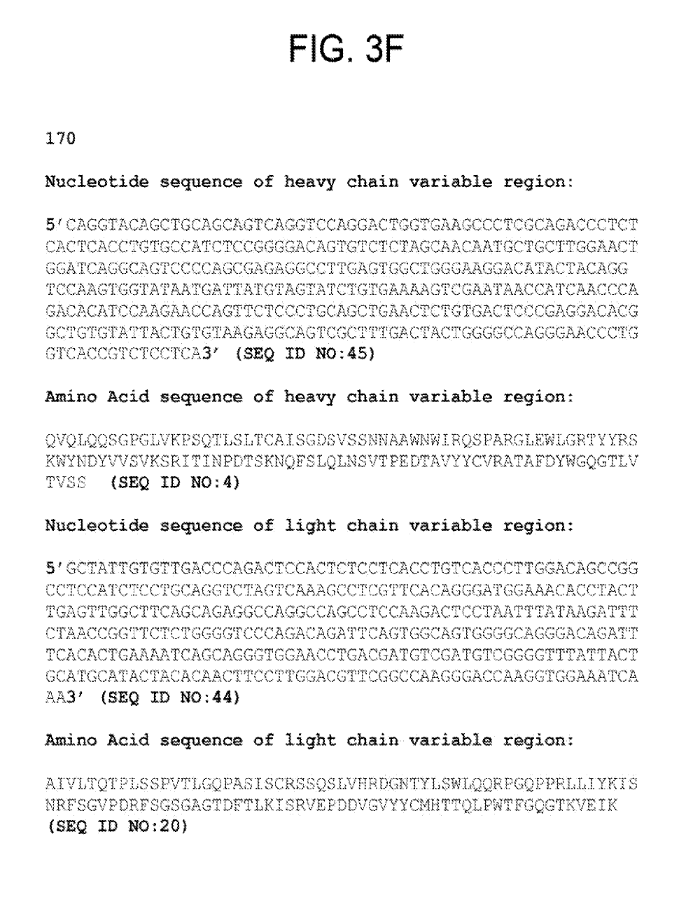

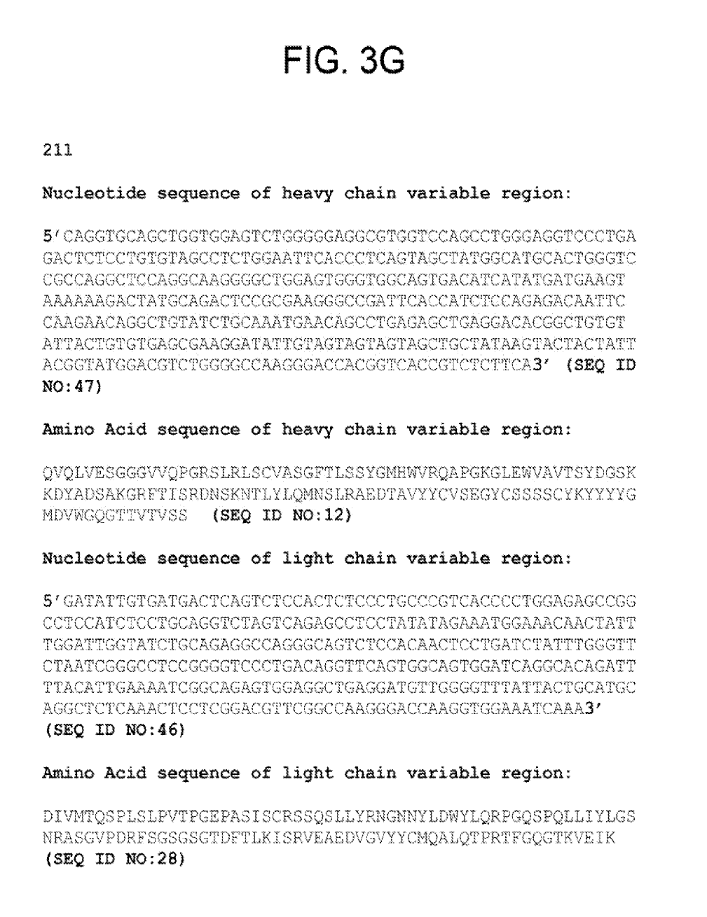

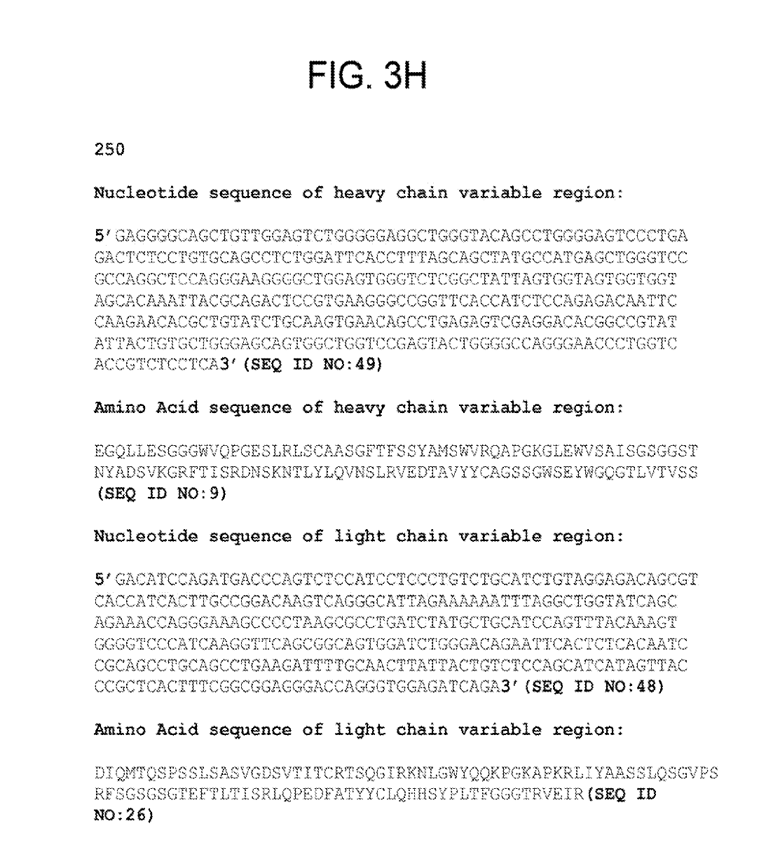

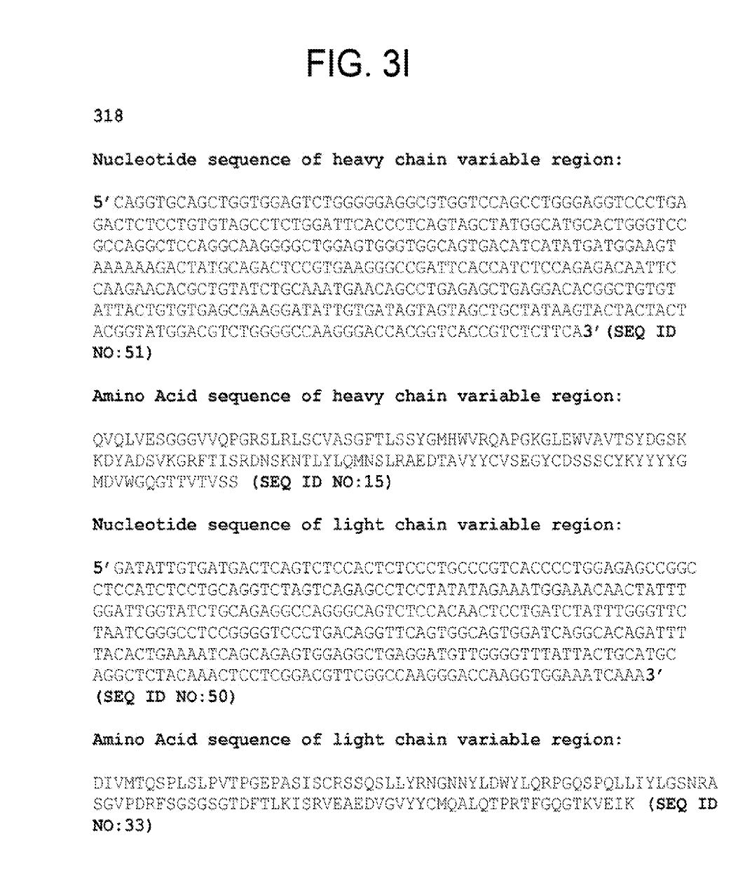

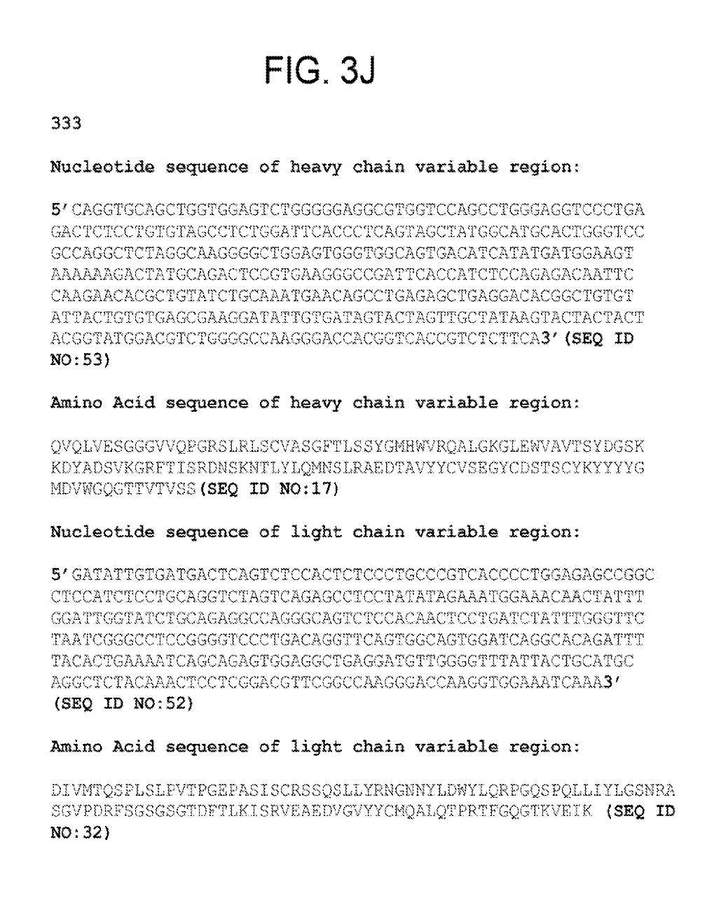

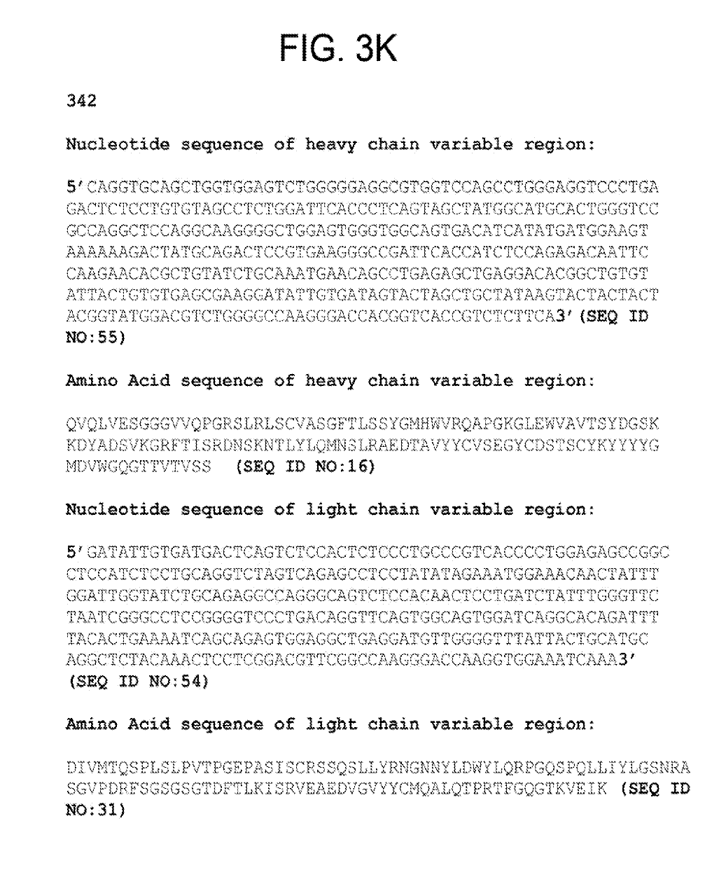

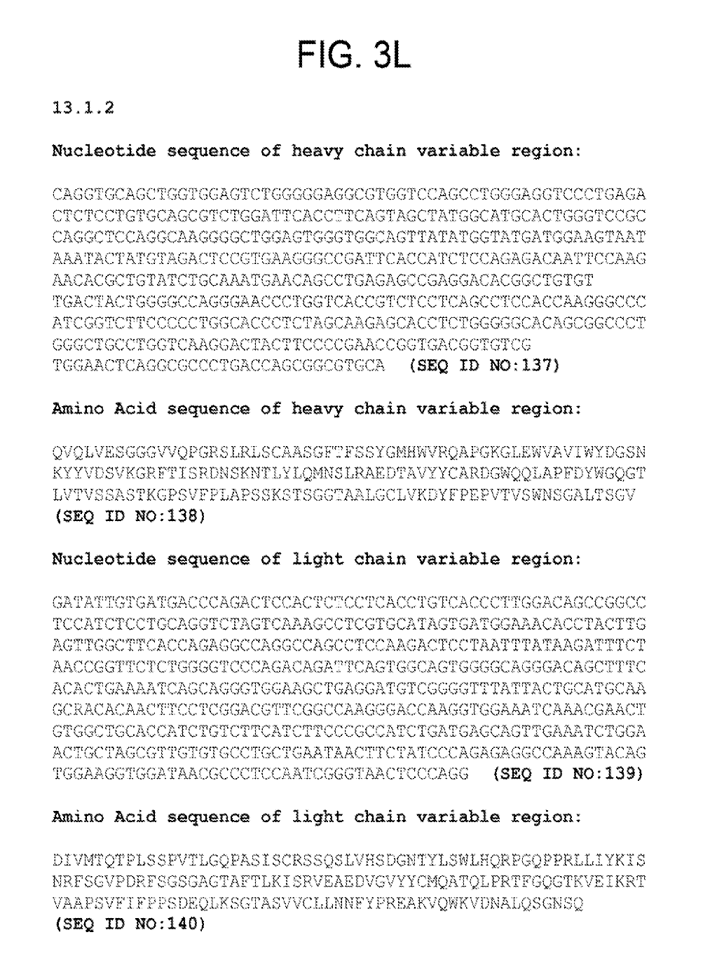

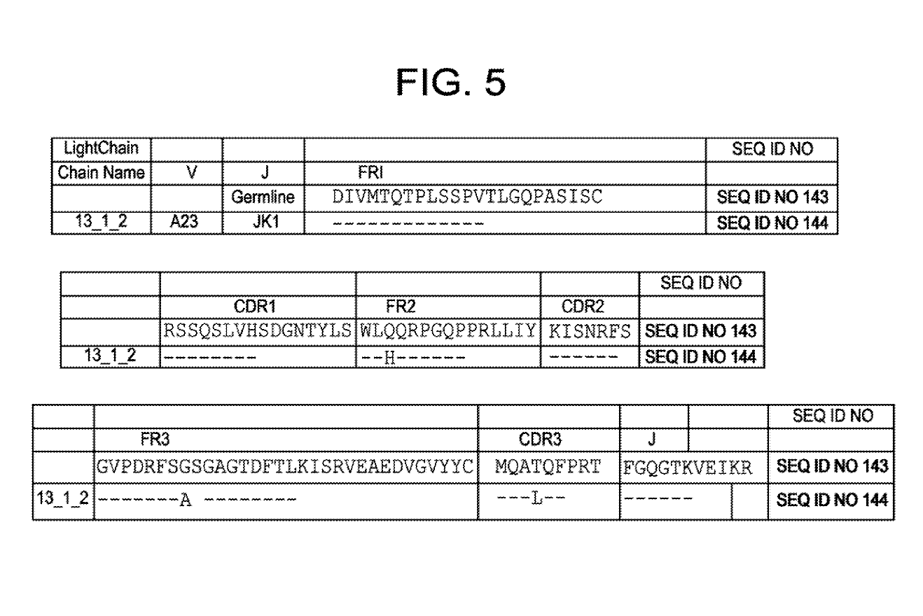

Cloning was performed on selected antigen-positive wells using limited dilution plating. Plates were visually inspected for the presence of single colony growth and supernatants from single colony wells then screened by antigen-specific ELISAs and FACS confirmation as described above. Highly reactive clones were assayed to verify purity of human gamma and kappa chain by multiplex ELISA using a Luminex instrument. Based on EGFRvIII specificity in the ELISA and FACS assay, Clone 13.1.2 was selected as the most promising candidate for further screening and analysis. The nucleotide and amino acid sequences of the heavy and light chains of 13.1.2 antibody are shown in FIG. 3L and SEQ ID NO: 137 and 139 for heavy and light chain nucleic acids and 138 and 140 for heavy and light chain amino acid sequences. In addition, a comparison of the 13.1.2 heavy chain and light chain sequences with the germline sequence from which they were derived as shown in FIGS. 4 and 5.

### Example 3

### Antibody Generation Through Use of XenoMax Technology

### Immunization of XenoMouse Animals

Human monoclonal antibodies against human EGFRvIII were developed by sequentially immunizing XenoMouse mice that produce antibodies with a gamma-1 constant region (XenoMouse G1 mice), XenoMouse mice that produce antibodies with gamma-2 constant regions (XenoMouse XMG2 mice), and XenoMouse mice that produce antibodies with a gamma-4 constant region (XenoMouse G4 mice).

To generate mAbs by through XenoMax technology, cohorts of XenoMouse G1 and XMG2 mice were immunized with EGFRvIII PEP3 (Example 1A) and EGFRvIII-expressing 300.19 cells (Example 1B) or with bacterially expressed extracellular domain of EGFRvIII protein (EGFRvIII-ECD) (Dr. Bigner, Duke University) and EGFRvIII-expressing 300.19 cells or with EGFRvIII-Rabbit Fc fusion protein (EGFRvIII-RbFc) (Example 1C) and EGFRvIII-expressing 300.19 cells or with EGFRvIII-RbFc only via foot pad (FP), or via base of the tail by subcutaneous injection and intraperitoneum (BIP).

For footpad immunizations, the initial immunization was with or without 10×10⁶ EGFRvIII-expressing 300.19 cells and with or without 10 μg of EGFRvIII PEP3 or EGFRvIII-ECD or EGFRvIII-RbFc mixed 1:1 v/v with Titermax gold (Sigma, Oakville, ON) per mouse. The subsequent boosts were performed with half of the amount of immunogen used in the initial immunization. The first four boosts were done by taking the immunogen mixed with alum (Sigma, Oakville, ON), adsorbed overnight, per mouse as shown in the Table 3.1 below. This was followed by one injection with the respective immunogen in Titermax gold, one injection with alum and then a final boost of the immunogen in PBS as shown in Table 3.1. In particular, animals were immunized on days 0, 3, 7, 10, 14, 17, 21 and 24. The animals were bled on day 19 to obtain sera and determine the titer for harvest selection. The animals were harvested on Day 28.

TABLE 3.1
Footpad immunization schedule

| | | | | | | | | |
|---|---|---|---|---|---|---|---|---|
| Group # | 1 | 2 | 3 | 4 | 5 | 6 | 7 | 8 |
| # of animals | 5 | 5 | 5 | 5 | 5 | 5 | 5 | 5 |
| Mouse strain | XMG2 | XM3C-3 | XMG2 | XM3C-3 | XMG2 | XM3C-3 | XMG2 | XM3C-3 |

| Boost # | Adjuvant | Immunogen | Immunogen | Immunogen | Immunogen |
|---|---|---|---|---|---|
| 1^{st} | Titermax | EGFRvIII- | EGFRvIII- | EGFRvIII- | EGFRvIII-RbFc |
| | gold | 300.19 cells + | 300.19 cells + | 300.19 cells + | |
| | | PEP3-KLH | EGFRvIII-ECD | EGFRvIII-RbFc | |
| 2^{nd} | Alum | EGFRvIII- | EGFRvIII- | EGFRvIII- | EGFRvIII-RbFc |
| | | 300.19 cells | 300.19 cells | 300.19 cells | |
| 3^{rd} | Alum | PEP3-KLH | EGFRvIII-ECD | EGFRvIII-ECD | EGFRvIII-RbFc |
| 4^{th} | Alum | EGFRvIII- | EGFRvIII- | EGFRvIII- | EGFRvIII-RbFc |
| | | 300.19 cells | 300.19 cells | 300.19 cells | |
| 5^{th} | Alum | PEP3-KLH | EGFRvIII-ECD | EGFRvIII-ECD | EGFRvIII-RbFc |
| 6^{th} | Titermax | EGFRvIII- | EGFRvIII- | EGFRvIII- | EGFRvIII-RbFc |
| | gold | 300.19 cells | 300.19 cells | 300.19 cells | |
| 7^{th} | Alum | PEP3-KLH | EGFRvIII-ECD | EGFRvIII-ECD | EGFRvIII-RbFc |
| 8^{th} | PBS | EGFRvIII- | EGFRvIII- | EGFRvIII- | EGFRvIII-RbFc |
| | | 300.19 cells + | 300.19 cells + | 300.19 cells + | |
| | | PEP3-KLH | EGFRvIII-ECD | EGFRvIII-RbFc | |

Harvest

The initial BIP immunization with the respective immunogen, as described for the footpad immunization, was mixed 1:1 v/v with Complete Freund's Adjuvant (CFA, Sigma, Oakville, ON) per mouse. Subsequent boosts were made first with the immunogen respectively, mixed 1:1 v/v with Incomplete Freund's Adjuvant (IFA, Sigma, Oakville, ON) per mouse, followed by a final boost in PBS per mouse. The animals were immunized on days 0, 14, 28, 42, 56, and day 75 (final boost) as shown in Table 3.2 below. The animals were bled on day 63 to obtain sera and determine the titer for harvest selection. The animals were harvested on Day 78.

TABLE 3.2
Bip Immunization schedule

| | | | | | | | | |
|---|---|---|---|---|---|---|---|---|
| Group | 9 | 10 | 11 | 12 | 13 | 14 | 15 | 16 |
| # of animals | 5 | 5 | 5 | 5 | 5 | 5 | 5 | 5 |
| Mouse strain | XMG2 | XM3C-3 | XMG2 | XM3C-3 | XMG2 | XM3C-3 | XMG2 | XM3C-3 |

| Boost # | Adjuvant | Immunogen | Immunogen | Immunogen | Immunogen |
|---|---|---|---|---|---|
| 1^{st} | CFA | EGFRvIII- | EGFRvIII- | EGFRvIII- | EGFRvIII-RbFc |
| | | 300.19 cells + | 300.19 cells + | 300.19 cells + | |
| | | PEP3-KLH | EGFRvIII-ECD | EGFRvIII-RbFc | |
| 2^{nd} | IFA | EGFRvIII- | EGFRvIII- | EGFRvIII- | EGFRvIII-RbFc |
| | | 300.19 cells | 300.19 cells | 300.19 cells | |
| 3^{rd} | IFA | PEP3-KLH | EGFRvIII-ECD | EGFRvIII-ECD | EGFRvIII-RbFc |
| 4^{th} | IFA | EGFRvIII- | EGFRvIII- | EGFRvIII- | EGFRvIII-RbFc |
| | | 300.19 cells | 300.19 cells | 300.19 cells | |
| 5^{th} | IFA | PEP3-KLH | EGFRvIII-ECD | EGFRvIII-ECD | EGFRvIII-RbFc |
| 6^{th} | PBS | EGFRvIII- | EGFRvIII- | EGFRvIII- | EGFRvIII-RbFc |
| | | 300.19 cells + | 300.19 cells + | 300.19 cells + | |
| | | PEP3-KLH | EGFRvIII-ECD | EGFRvIII-RbFc | |

Harvest

### Selection of Animals for Harvest by Titer Determination

Anti-hEGFRvIII antibody titers were determined by ELISA. EGFRvIII-RbFc (2.5 μg/ml) or a control RbFc (2 μg/ml) or EGFRvIIIpeptide-OVA (2 μg/ml) (Example 1) or control OVA (4 μg/ml) were coated onto Costar Labcoat Universal Binding Polystyrene 96-well plates (Corning, Acton, Mass.) overnight at four degrees. The solution containing unbound antigen was removed and the plates were treated with UV light (365 nm) for 4 minutes (4000 microjoules). The plates were washed five times with dH₂O. Sera from the EGFRvIII immunized XenoMouse® animals, or naïve XenoMouse® animals, were titrated in 2% milk/PBS at 1:2 dilutions in duplicate from a 1:100 initial dilution. The last well was left blank. The plates were washed five times with dH₂O. A goat anti-human IgG Fc-specific horseradish peroxidase (HRP, Pierce, Rockford, Ill.) conjugated antibody was added at a final concentration of 1 μg/mL for 1 hour at room temperature. The plates were washed five times with dH₂O. The plates were developed with the addition of TMB chromogenic substrate (Gaithersburg, Md.) for 30 minutes and the ELISA was stopped by the addition of 1 M phosphoric acid. The specific titer of individual XenoMouse® animals was determined from the optical density at 450 nm and is shown in Tables 3.3 and 3.4. The titer represents the reciprocal dilution of the serum and therefore the higher the number the greater the humoral immune response to hEGFRvIII.

For the mice immunized via base of the tail by subcutaneous injection and intraperitoneum, the titre was determined exactly as above except the plates were coated with EGFRvIII-RbFc (2.0 μg/ml) or a control RbFc (2.5 μg/ml).

TABLE 3.3

| | | | | | | EGFRvIII | |
|---|---|---|---|---|---|---|---|
| | Immunization | Mouse | | EGFRvIII- | Control | peptide- | OVA |
| Group | (site and | Strain | Mouse | RbFc @ | RbFc @ | OVA coated | coated at |
| # | Immunogen) | and sex | I.Ds | 2.5 ug/ml. | 2.0 ug/ml. | at 2.0 μg/ml | 4.0 μg/ml |
| 1 | FP | XMG2 | 0748-1 | 330 | | 13549 | <100 |
| | EGFRvIII- | | 0748-2 | 237 | | 7635 | <100 |
| | 300.19 cells + | | 0748-3 | 109 | | 9824 | <100 |
| | EGFRvIII | | 0748-4 | 714 | | 8014 | <100 |
| | PEP3-KLH (see | | 0748-5 | 165 | | 9421 | <100 |
| | Imm. Sched.) | | Naïve | <100 | | n/a | n/a |
| 2 | FP | XM3C-3 | 0741-1 | 388 | | 347 | <100 |
| | EGFRvIII- | | 0741-2 | 327 | | 240 | <100 |
| | 300.19 cells + | | 0741-3 | 385 | | 330 | <100 |
| | EGFRvIII | | 0741-4 | 589 | | 227 | <100 |
| | PEP3-KLH (see | | 0741-5 | 273 | | 626 | <100 |
| | Imm. Sched.) | | Naïve | <100 | | n/a | n/a |
| 3 | FP | XMG2 | 0749-1 | 552 | | <100 | <100 |
| | EGFRvIII- | | 0749-2 | 477 | | <100 | <100 |
| | 300.19 cells + | | 0749-3 | 100 | | <100 | <100 |
| | EGFRvIII- | | 0749-4 | 100 | | <100 | <100 |
| | ECD (see | | 0749-5 | 1631 | | <100 | <100 |
| | Imm. Sched.) | | Naïve | 100 | | n/a | n/a |
| 4 | FP | XM3C-3 | 0742-1 | 372 | | <100 | <100 |
| | EGFRvIII- | | 0742-2 | 745 | | <100 | <100 |
| | 300.19 cells + | | 0742-3 | 484 | | <100 | <100 |
| | EGFRvIII- | | 0742-4 | 530 | | <100 | <100 |
| | ECD (see | | 0742-5 | 270 | | <100 | <100 |
| | Imm. Sched.) | | Naïve | 100 | | n/a | n/a |
| 5 | FP | XMG2 | 0750-1 | 5399 | 175 | <100 | <100 |
| | EGFRvIII- | | 0750-2 | 3072 | 151 | <100 | <100 |
| | 300.19 cells + | | 0750-3 | >6400 | 358 | <100 | <100 |
| | EGFRvIII- | | 0750-4 | 5845 | 196 | <100 | <100 |
| | RbFc (see | | 0750-5 | 5770 | 196 | <100 | <100 |
| | Imm. Sched.) | | Naïve | 100 | 100 | n/a | n/a |
| 6 | FP | XM3C-3 | 0743-1 | 1220 | <100 | <100 | <100 |
| | EGFRvIII- | | 0743-2 | 1183 | <100 | <100 | <100 |
| | 300.19 cells + | | 0743-3 | 645 | <100 | <100 | <100 |
| | EGFRvIII- | | 0743-4 | 759 | <100 | <100 | <100 |
| | RbFc (see | | 0743-5 | 1260 | <100 | <100 | <100 |
| | Imm. Sched.) | | Naïve | 100 | <100 | n/a | n/a |
| 7 | FP | XMG2 | 0745-1 | 1897 | <100 | <100 | <100 |
| | EGFRvIII- | | 0745-2 | >6400 | 323 | <100 | <100 |
| | RbFc (see | | 0745-3 | 1225 | <100 | <100 | <100 |
| | Imm. Sched.) | | 0745-4 | 4047 | <100 | <100 | <100 |
| | | | 0745-5 | 852 | <100 | <100 | <100 |
| | | | Naïve | 100 | <100 | n/a | n/a |
| 8 | FP | XM3C-3 | 0744-1 | 362 | <100 | <100 | <100 |
| | EGFRvIII- | | 0744-2 | 807 | <100 | <100 | <100 |
| | RbFc (see | | 0744-3 | 479 | <100 | <100 | <100 |
| | Imm. Sched.) | | 0744-4 | 631 | <100 | <100 | <100 |
| | | | 0744-5 | 1112 | <100 | <100 | <100 |
| | | | Naïve | 100 | <100 | n/a | n/a |

All the XenoMouse animals from group 5 and XenoMouse animals 0743-5 from group 6 from Table 3.3 were selected for XenoMax harvests based on the serology.

TABLE 3.4

| | | | | | | EGFRvIII | |
|---|---|---|---|---|---|---|---|
| | Immunization | Mouse | | EGFRvIII- | Control | peptide- | OVA |
| Group | (site and | Strain | Mouse | RbFc @ | RbFc @ | OVA coated | coated at |
| # | Immunogen) | and sex | I.Ds | 2.0 ug/ml. | 2.5 ug/ml. | at 2.0 μg/ml | 4.0 μg/ml |
| 9 | BIP | XMG2 | O695-1 | 2921 | | >128000 | 472 |
| | EGFRvIII- | | O695-2 | 2219 | | 30504 | 379 |
| | 300.19 cells + | | O695-3 | 4609 | | >128000 | 608 |
| | EGFRvIII | | O695-4 | >6400 | | >128000 | 368 |
| | PEP3-KLH (see | | O695-5 | 1580 | | 19757 | 269 |
| | Imm. Sched.) | | Naïve | <100 | | n/a | 242 |
| 10 | BIP | XM3C-3 | O700-1 | <100 | | | |
| | EGFRvIII- | | O700-2 | <100 | | | |
| | 300.19 cells + | | O700-3 | >6400 | | | |
| | EGFRvIII | | O700-4 | 5342 | | | |
| | PEP3-KLH (see | | O700-5 | >6400 | | | |
| | Imm. Sched.) | | Naïve | <100 | | | |
| 11 | BIP | XMG2 | O696-1 | <100 | | 561 | 240 |
| | EGFRvIII- | | O696-2 | <100 | | 788 | 326 |
| | 300.19 cells + | | O696-3 | <100 | | 604 | 266 |
| | EGFRvIII- | | O696-4 | 143 | | 444 | 263 |
| | ECD (see | | O696-5 | <100 | | 303 | 254 |
| | Imm. Sched.) | | Naïve | <100 | | | 242 |
| 12 | BIP | XM3C-3 | O702-1 | 358 | | | |
| | EGFRvIII- | | O702-2 | 469 | | | |
| | 300.19 cells + | | O702-3 | 401 | | | |
| | EGFRvIII- | | O702-4 | >6400 | | | |
| | ECD (see | | O702-5 | >6400 | | | |
| | Imm. Sched.) | | Naïve | <100 | | | |
| 13 | BIP | XMG2 | O694-1 | >6400 | >6400 | 250 | 243 |
| | EGFRvIII- | | O694-2 | >6400 | >6400 | 296 | 309 |
| | 300.19 cells + | | O694-3 | >6400 | >6400 | 736 | 605 |
| | EGFRvIII- | | O694-4 | >6400 | >6400 | 739 | 1111 |
| | RbFc (see | | O694-5 | 3710 | >6400 | 517 | 465 |
| | Imm. Sched.) | | Naïve | <100 | >6400 | | 242 |
| 14 | BIP | XM3C-3 | O703-1 | 2740 | >6400 | | |
| | EGFRvIII- | | O703-2 | 408 | >6400 | | |
| | 300.19 cells + | | O703-3 | 1406 | >6400 | | |
| | EGFRvIII- | | O703-4 | 1017 | >6400 | | |
| | RbFc (see | | O703-5 | 403 | >6400 | | |
| | Imm. Sched.) | | Naïve | <100 | >6400 | | |
| 15 | BIP | XMG2 | O697-1 | >6400 | >6400 | 340 | 348 |
| | EGFRvIII- | | O697-2 | >6400 | >6400 | 642 | 1793 |
| | RbFc (see | | O697-3 | 6242 | >6400 | 319 | 246 |
| | Imm. Sched.) | | O697-4 | 1766 | >6400 | 133 | <100 |
| | | | O697-5 | >6400 | >6400 | 685 | 448 |
| | | | Naïve | <100 | >6400 | 243 | 242 |
| 16 | BIP | XM3C-3 | O701-1 | 592 | >6400 | | |
| | EGFRvIII- | | O701-2 | 1118 | >6400 | | |
| | RbFc (see | | O701-3 | >6400 | >6400 | | |
| | Imm. Sched.) | | O701-4 | <100 | <100 | | |
| | | | O701-5 | n/a | n/a | | |
| | | | Naïve | <100 | >6400 | | |

XenoMouse animals (0695-1, 0695-3 and 0695-4) were selected for harvests based on the serology data in Table 3.4.

### Selection of B Cells.

B-cells from the above-discussed animals were harvested and cultured. Those secreting EGFRvIII-peptide specific antibodies were isolated as described in Babcook et al., Proc. Natl. Acad. Sci. USA, 93:7843-7848 (1996). ELISA was used to identify primary EGFRvIII-peptide-OVA-specific wells. About 5 million B-cells were cultured from XenoMouse animals in 24596 well plates at 500 or 150 or 50 cells/well, and were screened on EGFRvIII-peptide-OVA to identify the antigen-specific wells. About 515 wells showed ODs significantly over background, a representative sample of which are shown in Table 3.5.

TABLE 3.5

| | Total | | | | | | | | | | | | | | | | |
|---|---|---|---|---|---|---|---|---|---|---|---|---|---|---|---|---|---|
| | # of | Positives above cutoff OD of: | | | | | | | | | | | | | | | |
| | plates | 0.0 | 0.1 | 0.2 | 0.3 | 0.4 | 0.5 | 0.6 | 0.7 | 0.8 | 0.9 | 1.0 | 1.5 | 2.0 | 2.5 | 3.0 | 3.5 |
| Cansera | 12 | 1152 | 634 | 81 | 56 | 49 | 45 | 38 | 32 | 29 | 26 | 25 | 18 | 11 | 4 | 1 | 0 |
| 500 | | | | | | | | | | | | | | | | | |
| cells/well | | | | | | | | | | | | | | | | | |
| Sigma | 13 | 1248 | 773 | 195 | 139 | 117 | 99 | 80 | 73 | 58 | 53 | 49 | 21 | 9 | 5 | 1 | 0 |
| 500 | | | | | | | | | | | | | | | | | |
| cells/well | | | | | | | | | | | | | | | | | |
| Sigma | 20 | 1920 | 1304 | 478 | 178 | 91 | 67 | 55 | 47 | 45 | 36 | 33 | 19 | 9 | 5 | 2 | 0 |
| 150 | | | | | | | | | | | | | | | | | |
| cells/well | | | | | | | | | | | | | | | | | |
| Total | 45 | 4320 | 2711 | 754 | 373 | 257 | 211 | 173 | 152 | 132 | 115 | 107 | 58 | 29 | 14 | 4 | 0 |

244 of EGFRvIII-peptide-OVA-Elisa positive wells of OD >0.5 were screened again on EGFRvIII-peptide-OVA and on OVA to confirm that they were EGFRvIII-peptide specific. A representative example of these results is shown in Table 3.6.

TABLE 3.6

| | | | 1′ EGFRvIII | 2′ EGFRvIII | |
|---|---|---|---|---|---|
| | | | peptide-OVA | peptide-OVA | OVA |
| Plate | Well | | OD | OD | OD |
| 121 | G | 1 | 0.7534 | 1.4065 | 0.1355 |
| 121 | A | 7 | 1.3472 | 2.1491 | 0.1268 |
| 121 | D | 8 | 0.6743 | 0.4179 | 0.1531 |
| 121 | E | 8 | 2.0415 | 2.6965 | 0.1498 |
| 121 | H | 10 | 0.8611 | 0.4288 | 0.1595 |
| 121 | C | 12 | 2.1455 | 2.6443 | 0.1404 |
| 122 | H | 1 | 1.8890 | 2.5987 | 0.1164 |
| 122 | H | 5 | 0.5943 | 0.8321 | 0.1572 |
| 122 | F | 8 | 0.6834 | 0.7715 | 0.1450 |

Limited Antigen Assay and Analysis

The limited antigen analysis is a method that affinity-ranks the antigen-specific antibodies present in B-cell culture supernatants relative to all other antigen-specific antibodies. In the presence of a very low coating of antigen, only the highest affinity antibodies should be able to bind to any detectable level at equilibrium. (See, e.g., International Patent Application No. WO 03/48730)

EGFRvIII peptide-OVA was coated to plates at three concentrations; 7.5 ng/ml, 1.5 ng/ml and 0.03 ng/ml for overnight at 4° C. on 96-well Elisa plates. Each plate was washed 5 times with dH₂O, before 50 ul of 1% milk in PBS with 0.05% sodium azide were added to the plate, followed by 4 μl of B cell supernatant added to each well. After 18 hours at room temperature on a shaker, the plates were again washed 5 times with dH₂O. To each well was added 50 ul of Gt anti-Human (Fc)-HRP at 1 μg/ml. After 1 hour at room temperature, the plates were again washed 5 times with dH₂O and 50 μL of TMB substrate were added to each well. The reaction was stopped by the addition of 50 uL of 1M phosphoric acid to each well and the plates were read at wavelength 450 nm and the results shown in Table 3.7.

TABLE 3.7

| | | | | | | | | | High |
|---|---|---|---|---|---|---|---|---|---|
| Cul- | | | Limited Ag | | | | | | Antigen |
| ture | | | 0.03 ng/ml | | 1.5 ng/ml | | 7.5 ng/ml | | (1.0 |
| Plate | Well | | OD | Rank | OD | Rank | OD | Rank | μg/ml) |
| 133 | B | 2 | 0.7670 | 1 | 1.189 | 54 | 1.871 | 95 | 2.050 |
| 124 | G | 12 | 0.7400 | 2 | 1.895 | 1 | 3.101 | 1 | 3.463 |
| 145 | C | 1 | 0.715 | 3 | 1.552 | 7 | 2.671 | 10 | 3.194 |
| 129 | G | 10 | 0.6720 | 4 | 1.367 | 22 | 2.692 | 8 | 2.977 |
| 186 | B | 6 | 0.657 | 5 | 1.842 | 2 | 2.859 | 3 | 3.411 |
| 143 | F | 12 | 0.653 | 6 | 1.677 | 3 | 2.741 | 6 | 3.156 |
| 136 | E | 3 | 0.6340 | 7 | 1.468 | 15 | 2.683 | 9 | 3.280 |
| 137 | C | 11 | 0.595 | 8 | 1.582 | 5 | 2.94 | 2 | 3.444 |
| 139 | A | 11 | 0.582 | 9 | 1.374 | 19 | 2.282 | 47 | 2.255 |
| 174 | F | 1 | 0.573 | 10 | 1.577 | 6 | 2.775 | 4 | 2.364 |

The results generated from limited antigen analysis were compared to the total OD obtained in high antigen assay. A relative ranking of affinity was done by taking the ratio of the OD obtained in limited antigen assay Vs that obtained in high antigen assay. Antibodies with higher ratio will have the highest affinity. Table 3.7 shows the sample of B-cell culture supernatants that were ranked based on limited antigen assay OD (for the lowest antigen plating concentration of 0.03 ng/ml) Vs the high antigen assay OD.

### Native Cell Binding Assay by FMAT

EGFRvIII peptide-OVA-Elisa positive well supernatants were analyzed for their ability to bind to the native form of EGFRvIII stably expressed on NR6 cells (NR6 M cells) (See, Batra et al. Epidermal growth factor ligand-independent, unregulated, cell-transforming potential of a naturally occurring human mutant EGFRvIII gene. Cell Growth Differ. 6(10):1251-9 (1995)). NR 6 M cells were seeded at 8000 cells per well and incubated over night in 96 well FMAT plates. Media was then removed leaving 15 μl in the well. 15 μl B-cell culture supernatants were added and 15 μl anti-human IgG Fc Cy5 at 1 μg/ml final concentration added to wells. It is then left incubated at 4° C. for 2 hours. The cells were washed with 150 μl PBS, and fixed before reading on FMAT. The results were expressed as total fluorescent intensity (Table 3.8). Human anti-EGFRvIII mAb 13.1.2 was used as a positive control starting at 1 μg/ml final concentration and negative control was PK 16.3.1 at the same concentration. 134 of the 244 samples tested bound to NREM cells of which 62 had a total fluorescence of greater than 8000. 6 of these 134 binders were false positives.

The same type of native binding assay was done on NR6 Wt cells (NR6 cells expressing EGF receptor) (See Batra et al. Epidermal growth factor ligand-independent, unregulated, cell-transforming potential of a naturally occurring human mutant EGFRvIII gene. Cell Growth Differ. 6(10):1251-9 (1995)) to eliminate the binding is due to binding to Wt receptor (Table 3.8). ABX-EGF was used as a positive control and PK 16.3.1 at the same concentration was used as a negative control antibody. 3 out the 134 NR6 M binders were binding strongly to NR6 Wt cells. 190 of the 244 wells bound EGFRvIII peptide in Elisa were also bound to the native form on cells. Examples are given in Table 3.8.

TABLE 3.8

| | | | | | | FMAT | FMAT |
|---|---|---|---|---|---|---|---|
| | | | | | | native | native |
| | | | 1′ VIII- | 2′ VIII- | | binding | binding |
| | | | pep-OVA | pep-OVA | OVA | to NR6 | to NR6 |
| Plate | | | OD | OD | OD | M cells | Wt cells |
| 174 | F | 1 | 2.4945 | 3.0308 | 0.1900 | 138373 | 1668 |
| 187 | A | 4 | 1.5337 | 1.2085 | 0.1920 | 128626 | 202459.8 |
| 132 | D | 8 | 0.8555 | 1.2070 | 0.1649 | 109379 | 0 |
| 142 | C | 11 | 2.2889 | 2.8194 | 0.2239 | 94944 | 0 |
| 129 | A | 7 | 2.1501 | 2.8208 | 0.1515 | 84024 | 0 |
| 127 | E | 1 | 2.6923 | 3.1986 | 0.1219 | 82031 | 0 |
| 124 | G | 12 | 3.2929 | 3.5634 | 0.1455 | 73080 | 0 |
| 141 | C | 6 | 0.7512 | 1.2567 | 0.1547 | 60816 | 814.5 |
| 173 | C | 1 | 2.5728 | 2.5714 | 0.2134 | 58702 | 2523.4 |
| 128 | G | 9 | 0.6293 | 0.7483 | 0.1520 | 49631 | 0 |
| 129 | H | 6 | 2.9370 | 3.0952 | 0.2582 | 0 | 0 |
| 183 | E | 11 | 2.3450 | 2.7717 | 0.1050 | 0 | 0 |

In Table 3.8, supernatant from well 187A4 is identified as a Wt binder and 14106 was a false positive for NR6 M cells binding. Wells 129H6 and 183E11 are strong peptide binders with no native binding.

### Internalization Assay

The top 60 native binding B cell culture supernatants were further assayed for their ability to internalize the receptor. NR6 M cells were seeded at 8000 cells/well into 96 well FMAT plates and incubated overnight. Media was removed and 10-15 μl B-Cell culture supernatant in a total volume of 30 μl media, in duplicate was added. Next, 15 μl of secondary antibody (SS Alexa 647 anti-human IgG Fab at 1.5 μg/ml final concentration) was added and the mixture was incubated on ice for 1 hr. An irrelevant B-Cell Culture supernatant was used to see the effect of the culture media. Human anti-EGFRvIII mAb 13.2.1 was used as a positive control starting at 1 μg/ml (final concentration) and negative control was PK 16.3.1 (human anti-KLH IgG2 antibody) at the same concentration. After incubation, the cells were washed with cold PBS, 50 μl media was added to all of the wells, one of the duplicates were incubated at 37° C. for 30 mins while the other duplicate remained on ice. After the incubations media was removed, 100 ul of cold 50 mM glutathione was added to the set incubated at 37° C. and 100 μl of cold media added to the other set, both sets were then left on ice for 1 hr. The cells were then washed with 100 μl cold PBS and then fixed with 1% paraformaldehyde and read in FMAT. The results were expressed as % internalized, calculated as total fluorescence in the presence of glutathione/total fluorescence in the absence of glutathione×100. Representative information is given in Table 3.9.

TABLE 3.9

| | | | % |
|---|---|---|---|
| | No | With | internalized, |
| | glutathione | glutathione | (glut+/glut−) × |
| Well no. | FL1xcount | FL1xcount | 100 |
| 124 C9 | 1877 | 1394 | 74.3% |
| 124 G12 | 26465 | 9959 | 37.6% |
| 125 H1 | 14608 | 3686 | 25.2% |
| 125 D10 | 2342 | 1236 | 52.8% |
| 127 E1 | 15059 | 1318 | 8.7% |
| 127 B9 | 12444 | 7109 | 57.1% |
| 127 E11 | 6623 | 0 | 0.0% |
| 128 G9 | 10071 | 1851 | 18.4% |
| 129 A7 | 27648 | 8708 | 31.5% |
| 130 B4 | 4558 | 4354 | 95.5% |
| 131 H5 | 9258 | 2656 | 28.7% |
| 132 D8 | 35820 | 13293 | 37.1% |
| 133 F9 | 9773 | 3621 | 37.0% |
| 136 F10 | 2392 | 0 | 0.0% |
| 137 G6 | 5104 | 1021 | 20.0% |
| 137 G10 | 3451 | 0 | 0.0% |

### EGFRvIII-Specific Hemolytic Plaque Assay.

A number of specialized reagents were needed to conduct this assay. These reagents were prepared as follows.

1. Biotinylation of Sheep red blood cells (SRBC). SRBCs were stored in RPMI media as a 25% stock. A 250 μl SRBC packed-cell pellet was obtained by aliquoting 1.0 ml of SRBC to a fresh eppendorf tube. The SRBC were pelleted with a pulse spin at 8000 rpm (6800 rcf) in microfuge, the supernatant drawn off, the pellet re-suspended in 1.0 ml PBS at pH 8.6, and the centrifugation repeated. The wash cycle was repeated 2 times, then the SRBC pellet was transferred to a 15-ml falcon tube and made to 5 ml with PBS pH 8.6. In a separate 50 ml falcon tube, 2.5 mg of Sulfo-NHS biotin was added to 45 ml of PBS pH 8.6. Once the biotin had completely dissolved, the 5 ml of SRBCs were added and the tube rotated at RT for 1 hour. The SRBCs were centrifuged at 3000 rpm for 5 min and the supernatant drawn off. The biotinylated SRBCs were transferred to an eppendorf tube and washed 3 times as above but with PBS pH 7.4 and then made up to 5 ml with immune cell media (RPMI 1640) in a 15 ml falcon tube (5% B-SRBC stock). Stock was stored at 4° C. until needed.

2. Streptavidin (SA) coating of B-SRBC. 1 ml of the 5% B-SRBC stock was transferred into a fresh eppendorf tube. The B-SRBC cells were washed 3 times as above and resuspended in 1.0 ml of PBS at pH 7.4 to give a final concentration of 5% (v/v). 10 μl of a 10 mg/ml streptavidin (CalBiochem, San Diego, Calif.) stock solution was added and the tube mixed and rotated at RT for 20 min. The washing steps were repeated and the SA-SRBC were re-suspended in 1 ml PBS pH 7.4 (5% (v/v)).

3. EGFRvIII coating of SA-SRBC. The SA-SRBCs were coated with biotinylated-EGFRvIIIpetide-OVA at 10 μg/ml, mixed and rotated at RT for 20 min. The SRBC were washed twice with 1.0 ml of PBS at pH 7.4 as above. The EGFRvIII-coated SRBC were re-suspended in RPMI (+10% FCS) to a final concentration of 5% (v/v).

4. Determination of the quality of EGFRvIIIpeptide-SRBC by immunofluorescence (IF). 10 μl of 5% SA-SRBC and 10 μl of 5% EGFRvIII peptide-coated SRBC were each added to a separate fresh 1.5 ml eppendorf tube containing 40 ul of PBS. A control human anti-EGFRvIII antibody was added to each sample of SRBCs at 45 μg/ml. The tubes were rotated at RT for 25 min, and the cells were then washed three times with 100 μl of PBS. The cells were re-suspended in 50 μl of PBS and incubated with 40 mcg/mL Gt-anti Human IgG Fc antibody conjugated to Alexa488 (Molecular Probes, Eugene, Oreg.). The tubes were rotated at RT for 25 min, and then washed with 100 μl PBS and the cells re-suspended in 10 μl PBS. 10 μl of the stained cells were spotted onto a clean glass microscope slide, covered with a glass coverslip, observed under fluorescent light, and scored on an arbitrary scale of 0-4.

5. Preparation of plasma cells. The contents of a single microculture well previously identified by various assays as containing a B cell clone secreting the immunoglobulin of interest were harvested. Using a 100-1000 μl pipetman, the contents of the well were recovered by adding 37C RPMI (10% FCS). The cells were re-suspended by pipetting and then transferred to a fresh 1.5 ml eppendorf tube (final vol. approx 500-700 μl). The cells were centrifuged in a microfuge at 2500 rpm (660 rcf) for 1 minute at room temperature, and then the tube was rotated 180 degrees and spun again for 1 minute at 2500 rpm. The freeze media was drawn off and the immune cells resuspended in 100 μl RPMI (10% FCS), then centrifuged. This washing with RPMI (10% FCS) was repeated and the cells re-suspended in 60 μl RPMI (10% FCS) and stored on ice until ready to use.

6. Micromanipulation of plasma cells. Glass slides (2×3 inch) were prepared in advance with silicone edges and allowed to cure overnight at RT. Before use, the slides were treated with approx. 5 ul of SigmaCoat (Sigma, Oakville, ON) wiped evenly over glass surface, allowed to dry and then wiped vigorously. To a 60 μl sample of cells was added 60 μl each of EGFRvIIIpeptide-coated SRBC (5% v/v stock), 4× guinea pig complement (Sigma, Oakville, ON) stock prepared in RPMI (10% FCS), and 4× enhancing sera stock (1:150 in RPMI with 10% FCS). The mixture was spotted (10-15 μl) onto the prepared slides and the spots covered with undiluted paraffin oil. The slides were incubated at 37° C. for a minimum of 45 minutes. The EGFRvIII-specific plasma cells were identified from plaques and rescued by micromanipulation (see Table 3.10).

TABLE 3.10

| | | | | Total number of |
|---|---|---|---|---|
| Well ID | | | Single Cell Number | Single cells picked |
| 124 | G | 12 | EGFRvIII-SCX-105-116 (LL) | 12 |
| 129 | A | 7 | EGFRvIII-SCX-117-128 (DM) | 12 |
| 174 | F | 1 | EGFRvIII-SCX-129-137 (DM) | 9 |
| 182 | A | 5 | EGFRvIII-SCX-138-149 (LL); | 20 |
| | | | 162-169 (OP) | |
| 125 | D | 10 | EGFRvIII-SCX-170-181 (DM); | 20 |
| | | | 194-201 (LL) | |
| 127 | B | 9 | EGFRvIII-SCX-182-193 (LL); | 20 |
| | | | 202-209 (OP) | |
| 190 | D | 7 | EGFRvIII-SCX-210-229 (LL) | 20 |
| 130 | B | 4 | EGFRvIII-SCX-230-249 (LL) | 20 |
| 138 | D | 2 | EGFRvIII-SCX-250-269 (LL) | 20 |
| 145 | C | 1 | EGFRvIII-SCX-80-92 (DM) | 13 |
| 172 | B | 12 | EGFRvIII-SCX-93-104 (LL) | 12 |
| 187 | A | 4 | EGFRvIII-SCX-270-281 (LL) | 12 |
| 173 | C | 1 | EGFRvIII-SCX-282-293 (BC) | 12 |
| 127 | E | 1 | EGFRvIII-SCX-294-305 (LL) | 12 |
| 142 | C | 11 | EGFRvIII-SCX-306-317 (LL) | 12 |
| 141 | A | 10 | EGFRvIII-SCX-318-329 (BC) | 12 |
| 132 | D | 8 | EGFRvIII-SCX-330-341 (LL) | 12 |
| 124 | D | 4 | EGFRvIII-SCX-342-349 (BC) | 8 |

### Single Cell PCR, Cloning, Expression, Purification and Characterization of Recombinant Anti-EGFRvIII Antibodies.

The genes encoding the variable regions were rescued by RT-PCR on the single micromanipulated plasma cells. mRNA was extracted and reverse transcriptase PCR was conducted to generate cDNA. The cDNA encoding the variable heavy and light chains was specifically amplified using polymerase chain reaction. The human variable heavy chain region was cloned into an IgG 1 expression vector. This vector was generated by cloning the constant domain of human IgG1 into the multiple cloning site of pcDNA3.1+/Hygro (Invitrogen, Burlington, ON). The human variable light chain region was cloned into an IgK expression vector. These vectors were generated by cloning the constant domain of human IgK into the multiple cloning site of pcDNA3.1+/Neo (Invitrogen, Burlington, ON). The heavy chain and the light chain expression vectors were then co-lipofected into a 60 mm dish of 70% confluent human embryonal kidney 293 cells and the transfected cells were allowed to secrete a recombinant antibody with the identical specificity as the original plasma cell for 24-72 hours. The supernatant (3 mL) was harvested from the HEK 293 cells and the secretion of an intact antibody was demonstrated with a sandwich ELISA to specifically detect human IgG (Table 3.11). Specificity was assessed through binding of the recombinant antibody to EGFRvIII using ELISA (Table 3.11).

TABLE 3.11

| | | Titer | |
|---|---|---|---|
| | | Total | Antigen |
| mAb ID | Cell # | antibody | binding |
| 129A7 | SC-EGFRvIII-XG1-123/124 | >1:64 | >1:64 |
| 138D2 | SC-EGFRvIII-XG1-250 | >1:64 | >1:64 |
| 174F1 | SC-EGFRvIII-XG1-131 | >1:64 | >1:64 |
| 182A5 | SC-EGFRvIII-XG1-139 | >1:64 | >1:64 |
| 190D7 | SC-EGFRvIII-XG1-211 | >1:64 | >1:64 |
| 125D10 | SC-EGFRvIII-XG2-170 | >1:64 | >1:64 |
| 182D5 | SC-EGFRvIII-XG2-150 | >1:64 | >1:64 |
| 141A10 | SC-EGFRvIII-XG1-318 | 1:64 | 1:64 |
| 132D8 | SC-EGFRvIII-XG1-333 | >1:64 | >1:64 |
| 124D4 | SC-EGFRvIII-XG1-342 | >1:64 | >1:64 |

The secretion ELISA tests were performed as follows. For Ab secretion, 2 μg/mL of Goat anti-human IgG H+L and for antigen binding, 1.5 μg/ml of EGFRvIII-Rab Ig Fc fusion protein was coated onto Costar Labcoat Universal Binding Polystyrene 96 well plates and held overnight at four degrees. The plates were washed five times with dH₂O. Recombinant antibodies were titrated 1:2 for 7 wells from the undiluted minilipofection supernatant. The plates were washed five times with dH₂O. A goat anti-human IgG Fc-specific HRP-conjugated antibody was added at a final concentration of 1 μg/mL for 1 hour at RT for the secretion plates and binding plates detected with 1 μg/ml Rb anti Hu Fc for 1 hour at room temperature. The plates were washed five times with dH₂O. The plates were developed with the addition of TMB for 30 minutes and the ELISA was stopped by the addition of 1 M phosphoric acid. Each ELISA plate was analyzed to determine the optical density of each well at 450 nm.

### Sequencing and Sequence Analysis

The cloned heavy and light chain cDNAs were sequenced in both directions and analyzed to determine the germline sequence derivation of the antibodies and identify changes from germline sequence. Such sequences are provided in FIGS. 3A-3K and (SEQ ID NO: 34-55). A comparison of each of the heavy and light chain sequences and the germline sequences from which they are derived is provided in FIGS. 4-7. In addition, the sequence of the hybridoma derived 13.1.2 antibody is compared to its germline sequence in FIGS. 4 and 5.

As will be appreciated from the discussion herein, each of the 131 antibody and the 13.1.2 antibody possess very high affinities for EGFRvIII, are internalized well by cells, and appear highly effective in cell killing when conjugated to toxins. Intriguingly, each of the antibodies, despite having been generated in different immunizations of XenoMouse mice, and utilizing different technologies, each are derived from very similar germline genes. Based upon epitope mapping work (described herein), each of the antibodies, however, appear to bind to slightly different epitopes on the EGFRvIII molecule and have slightly different residues on EGFRvIII that are essential for binding. These results indicate that the germline gene utilization is of importance to generation of antibody therapeutics targeting EGFRvIII and that small changes can modify the binding and effects of the antibody in ways that allow further design of antibody and other therapeutics based upon these structural findings.

Binding of Anti-EGFRvIII mAbs to Native EGFRvIII Expressed on Cells

In this example, binding of anti-EGFRvIII antibodies to NR6 M cells was measured. Specifically, unquantitated supernatants of XenoMax derived IgG1 recombinant antibodies were assayed for their ability to bind to NR6 M and NR6 WT cells. Cells were seeded at 10000/well and incubated overnight at 37 C in FMAT 96 well plates. Media was removed and 40 μl mini lipo supernatant (titrated down) was added, the cells were incubated on ice for 1 hr. The human 13.1.2 EGFRvIII antibodies and ABX EGF (E7.6.3, U.S. Pat. No. 6,235,883) antibodies were added as positive controls. The PK 16.3.1 antibody was used as a negative control. The cells were washed with Cold PBS, secondary antibody was added (SS Alexa antihuman IgG Fc) at 1 μg/ml, 40 μl/well and incubated on ice for 1 hr. The cells were then washed with Cold PBS and fixed and read by FMAT. All antibodies were tested for specificity for binding by counter screening against NR6 WT cells.

### Purification of Recombinant Anti-EGFRvIII Antibodies.

For larger scale production, heavy and light chain expression vectors (2.5 μg of each chain/dish) were lipofected into ten 100 mm dishes that were 70% confluent with HEK 293 cells. The transfected cells were incubated at 37° C. for 4 days, the supernatant (6 mL) was harvested and replaced with 6 mL of fresh media. At day 7, the supernatant was removed and pooled with the initial harvest (120 mL total from 10 plates). Each antibody was purified from the supernatant using a Protein-A Sepharose (Amersham Biosciences, Piscataway, N.J.) affinity chromatography (1 mL). The antibody was eluted from the Protein-A column with 500 mcL of 0.1 M Glycine pH 2.5. The eluate was dialyzed in PBS, pH 7.4 and filter-sterilized. The antibody was analyzed by non-reducing SDS-PAGE to assess purity and yield. Concentration was also measured by UV analysis at OD 250.

Internalization of EGFRvIII Receptor by Recombinant Anti-EGFRvIII mAbs

XenoMax derived IgG1 recombinant antibodies were expressed, purified and quantitated as described previously. Antibodies were further assayed for their ability to internalize the EGFRvIII receptor in NR6 M cells. 250,000 NR6 M cells were incubated with primary antibody (SC95, SC131, SC133, SC139, SC150, SC170, SC211, SC230, SC250 and human 13.1.2 as a control) at 0.25 μg/ml, 7 mins on ice in 96 well v-bottomed plate in triplicate. The cells were washed with cold 10% FCS in PBS and secondary antibody (SS Alexa antihuman IgG Fab) at 3 μg/ml Fab was added and incubated for 7 mins on ice. The cells were washed with cold 10% FCS in PBS once and then resuspended in cold media. Next, two sets of the triplicate were incubated at 37° C. and the remaining set was incubated at 4° C. for 1 hr. After that the cells incubated at 4° C. and one set of the cells incubated at 37° C. were treated with glutathione (as previously mentioned) for 1 hr on ice. Then the cells were washed and resuspended in 100 μl of cold 1% FCS in PBS and analyzed by FACS. The % internalization was calculated from the geometric mean obtained from the FACS analysis [(mean at 37° C. with glutathione−mean at 4° C. with glutathione)/(mean at 37° C. without glutathione−mean at 4 C with glutathione)]. NA means that a FACS analysis was performed but the data was not provided in Table 3.12.

TABLE 3.12

| | FACS Geometric mean | | | |
|---|---|---|---|---|
| | Without | With | | |
| | glutathione | glutathione | With glutathione | % |
| mAb | 37° C. | 37° C. | 4° C. | internalization |
| 13.1.2 | 22.12 | 19.19 | 5.38 | 82.5% |
| sc95 | 22.56 | 17.75 | 5.13 | 72.4% |
| sc131 | NA | NA | NA | 72% |
| sc133 | 23.39 | 18.63 | 6.24 | 72.2% |
| sc139 | 22.64 | 19.23 | 4.88 | 80.8% |
| sc150 | 20.29 | 7.78 | 4.66 | 20.0% |
| sc170 | 19.97 | 7.75 | 4.67 | 20.1% |
| sc211 | 20.76 | 8.23 | 4.78 | 21.6% |
| sc230 | 20.68 | 7.97 | 5.02 | 18.8% |
| sc250 | 24.13 | 8.07 | 4.84 | 16.7% |

13.1.2 is an antibody that was generated through hybridoma generation (Example 2) that was directed against the EGFRvIII epitope previously and was used as a positive control in this experiment. These results in Table 3.12 demonstrate the presence of two subsets of antibodies, those that are efficiently internalized (70-80%) and those that are not (22% or less).

### Example 4

### Antibody 131-DM1 Conjugation

### Purification of 131 Antibody

131 antibody (comprising the heavy chain of FIG. 8B and the light chain of FIG. 8D), transiently expressed in mammalian cell culture 2936-E cells, was loaded onto a MabSelect SuRe column (GE Healthcare) that had been equilibrated in 25 mM Tris, 150 mM Sodium Chloride, pH 7.4. The column with bound 131 antibody was then washed with 3 wash steps: first an equilibration buffer wash, followed by a 25 mM Tris, 500 mM L-Arginine, pH 7.5 wash and a final wash with equilibration buffer. 131 antibody was eluted with 100 mM Sodium Acetate, pH 3.5. Fractions containing the antibody were pooled and adjusted to a final pH of 5.0 with 1M Tris, pH 8.0. The antibody was subsequently dialyzed into Conjugation Buffer (2 mM EDTA, 50 mM Sodium Chloride, 50 mM Potassium Phosphate, pH 6.5).

### Modification of 131 Antibody with SMCC

The purified 131 antibody was modified with the amine reactive linker Succinimidyl-4-(N-maleimidomethyl)cyclohexane-1-carboxylate (SMCC) (Thermo Scientific) to introduce thiol reactive maleimide groups. The antibody was treated with 12 molar equivalents of SMCC and incubated for 90 minutes at room temperature, the reaction mixture was desalted with a HiPrep 26/10 Desalting Column containing Sephadex G-25 fine resin (GE Healthcare).

### Conjugation of SMCC Linked 131 Antibody with DM1

The SMCC modified 131 antibody was treated with 1.7 molar equivalents of DM1 (Immunogen) per maleimide group buffered with 2 mM EDTA, 150 mM NaCl, 35 mM Sodium Citrate, pH 5.0 adjusted to 3% DMA (v/v) in the final reaction mixture. Reaction mixtures were incubated at room temperature overnight for up to 20 hours. The reaction mixture was loaded on a Superdex 200 gel filtration column (GE Healthcare) equilibrated with 20 mM Sodium Phosphate, 150 mM Sodium Chloride, pH 6.5. Fractions were collected and and monomeric antibody containing fractions pooled and assayed. The molar ratio of DM1 molecules linked per antibody was determined by measuring the absorbance at 252 nm and 280 nm, and for different conjugation lots was found to be between 3.0-3.5 DM1 molecules per antibody.

### Example 5

### Antibody 131-DM1 Binding Specificity

EGFR positive, EGFRvIII negative A431 cells and EGFRvIII positive U87vIII cells were isolated and counted. 1×10⁶ cells was added to each tube and washed. Cells were washed by adding FACS wash buffer (PBS, 2% fetal bovine serum, 0.02% sodium azide) to cells followed by centrifugation at 1800 rpm for 5 minutes, supernatant was discarded. Cells were incubated with primary antibody either anti-EGFRvIII antibody (131 antibody), Antibody 131-DM1 conjugate, anti-wild type EGFR antibody that also binds EGFRvIII or control IgG1 antibody for 1 hour at 4° C. Following incubation cells were washed and then incubated with secondary antibody anti-human IgG antibody Alexa Fluor 488 (Invitrogen, Carlsbad, Calif.) for 1 hour at 4° C. Cells were washed and resuspended in FACS wash buffer and analyzed using a FACS Calibur flow cytometer.

As illustrated by FIG. 9, Anti-EGFRvIII antibody (black dashed line), Ab 131-DM1 conjugate (solid black line), anti-EGFR antibody (gray dot dashed) all bind to EGFRvIII expressing cells. Neither anti-EGFRvIII antibody nor Ab 131-DM1 bind to EGFR (wild-type) overexpressing cells, in contrast to anti-EGFR antibody.

### Example 6

### Antibody 131-DM1 Internalization

U251vIII cells were plated on a 96-well plate at 10,000 cells per well with 200 μL of growth medium (DMEM containing 10% FBS) and incubated at 37° C., 5% CO₂ for 3 days to reach to 90% cell confluency on assay day. Ab 131-DM1 conjugate was added to cells at 5 μg/mL for 20 minutes at 4 C. Anti-human IgG Fab′ Alexa 488 (Nanoprobes, Yaphank, N.Y.) and Hoechst 33342 (Invitrogen, Carlsbad, Calif.) were added to cells in assay medium at 4° C. for 20 minutes. Cells were washed twice with assay medium and then incubated at 37° C. for 1.5 hours. Cells were fixed and permeabilized using BD cytofix/cytoperm kit (BD biosciences, San Diego, Calif.), briefly cells were washed once with BD wash buffer followed by addition of Fix/perm solution. Cells were incubated with anti-EEA1 antibody (BD Biosciences, San Diego, Calif.) at 0.5 μg/mL at RT for 20 minutes. Cells were washed with BD wash buffer and anti-mouse Alexa 568 (Invitrogen, Carlsbad, Calif.) was added to cells. Cells were incubated at RT for 20 minutes followed by two wash steps of BD buffer solution. Cell images for co-localization were taken with a Leica florescent microscope connected to Hamamutsu digital camera with Openlab Image Analysis software (Improvision Inc, Lexington, Mass.).

At 0 hour Ab 131-DM1 conjugate bound EGFRvIII receptors are visible on the surface of U251vIII cells. After a 1.5 hour incubation, Ab 131-DM1 conjugate was internalized from the cell surface into the cytoplasm and colocalized to endosomes.

### Example 7

### Ab 131-DM1 Conjugate Inhibits U251vIII Cell Growth

U251 and U251vIII cells were seeded to a 96-well tissue culture plate at 500 cells per well with 100 μL of growth medium (DMEM containing 10% FBS) and incubated at 37° C., 5% CO₂ for 4 hours. After a 4 hour incubation, dose titrations of either Ab, control conjugate, or media alone was added to cells. Cells were continuously incubated at 37° C., 5% CO₂ for 4 days prior to measurement of cellular ATP levels. CellTiter-Glo (Promega Corp., Madison, Wis.) buffer and substrate were equilibrated to room temperature (RT) for approximately 60 minutes. Plates were removed from the 37° C. incubator and incubated at RT for 30 minutes. CellTiter-Glo buffer was mixed with CellTiter-Glo substrate to generate CellTiter-Glo reagent. CellTiter-Glo reagent was added to each well of both plates at a 1:1 ratio of CellTiter-Glo reagent to media. Plates were placed on a plate shaker and shaken slowly for 2 minutes. Plates were incubated for 10 minutes prior to measuring luminescence. Luminescence was measured with Wallac EnVision 2103 multilabel reader (Perkin Elmer, Waltham, Mass.) with a reading time of 0.1 second per well. Statistical analysis was performed using Prism 4.01 (GraphPad, San Diego, Calif.). Luminescence results were plotted on the y-axis and the log of concentration in nM DM1 equivalents was plotted on the x-axis. The IC₅₀ value was determined from the dose response curve by using nonlinear regression analysis (sigmoidal curve fit) of log transformed concentration data.

As illustrated in FIG. 10, in EGFRvIII expressing U251vIII cells, Ab 131-DM1 led to significant cell growth inhibition with an IC₅₀ of 0.068 nM DM1 equivalents, or 3.0 ng/mL Ab 131-DM1. No cell growth inhibition was observed with the control the control conjugate.

### Example 8

### Ab 131-DM1 Conjugate Increased Phospho-Histone H3 Levels in D317 Xenografts

Animals bearing D317 human glioblastoma xenografts (passage 213) were euthanized and tumors removed under sterile conditions. Tumors were cut into similar sized fragments that fit into 13 gauge implant trocars. Tumors were implanted into the flanks of naïve CB-17/SCID mice for passage number 214. Tumor measurement: the length and width were measured with an electronic digital caliper. Tumor Volume (mm³)=[(W²×L)/2] where width (W) is defined as the smaller of the 2 measurements and length (L) is defined as the larger of the 2 measurements. Tumor volume was measured and on day fourteen post implantation the tumor volume ranged from 258 to 875 mm³ in 42 animals. Animals were subsequently randomized into groups of six animals each for treatment initiation. Treatment was administered intravenously via the tail vein on day fourteen. Animals were administered with either vehicle, Ab131-DM1 at 5.3 and 16.7 mg/kg (80 and 250 ug DM1/kg, respectively), or control conjugate at 17.8 mg/kg (250 ug DM1/kg). Animals were dosed at a volume of 10 mL/kg using group body weights. Tumors and animal weights were measured prior to euthanasia which occurred 40 hours post treatment administration.

Forty hours after administration, animals were euthanized to collect tumors from each animal, tumors were fixed in 10% neutral buffered formalin, processed routinely and embedded in paraffin blocks. For phosphohistoneH3 immunohistochemistry, 4-6 micron sections of each tumor were placed on charged glass slides, deparaffinized, and rehydrated prior to antigen retrieval using Diva solution (Biocare # DV2004G1) in the Decloaker Pressure Instrument (Biocare). Sections were incubated with anti-phosphohistoneH3 antibody (Millipore/Upstate #06-570) at 1 ug/ml for 1 hour at room temperature and binding was visualized with anti-rabbit Envision (Dako K4003) and DAB (Dako 3468) followed by counter staining with hematoxylin. The slides were digitally scanned using an Aperio ScanScope XT and the viable tumor within each section was manually outlined on the resulting image by a board certified veterinary pathologist blinded to treatment group using ImageScope software. The number of phosphohistone H3 positive cells within the viable tumor area was quantified using the IHC Nuclear algorithm and expressed as the number of positive cells per mm² of viable tumor area.

The average number of phospho-histone H3 positive cells per mm² in the treatment groups were compared to the number observed in the control groups using the Mann-Whitney test using GraphPad Prism version 5.04 for Windows (GraphPad Software, SanDiego, Calif.).

As illustrated in FIG. 11, treatment with Ab 131-DM1 led to a significant increase in the number of positive phospho-histone H3 cells compared to Vehicle or control conjugate treated animals.

### Example 9

### Ab 131-DM1 Collimate in Xenograft Models

In Examples 46A, 46B and 46C below, the length and width of tumors were measured with an electronic digital caliper. Tumor Volume (mm³) was calculated as [(W²×L)/2] where width (W) is defined as the smaller of the 2 measurements and length (L) is defined as the larger of the 2 measurements. Animals were dosed at a volume of 10 mL/kg using group body weights. Tumor volumes and animal weights were measured two or three times a week. Animals were euthanized as tumor burden approached 10% of body weight. Tumor volumes were expressed as means plus or minus standard errors and plotted as a function of time. Statistical significance of observed differences between growth curves was evaluated by repeated measures analysis of covariance of the log transformed tumor volume data with Dunnett adjusted multiple comparisons post hoc. The analysis was done using SAS proc mixed with model effects of baseline log tumor volume, day, treatment and day-by-treatment interaction; a repeated statement where day was a repeated value, animal the subject and a Toeplitz covariance structure; and an lsmeans statement to do a Dunnett analysis comparing the control group to the other treatment groups. The data was log transformed because larger volumes tended to have larger variances, and baseline log tumor volume was included as a covariate in the model to account for possible pre-treatment tumor volume differences. All statistical calculations were made through the use of JMP software v7.0 interfaced with SAS v9.1 (SAS Institute, Inc., Cary, N.C.).

### Example 9A

### Ab 131-DM1 Conjugate in U251vIII Xenograft Model

To determine the efficacy of Ab 131 in U251vIII subcutaneous xenografts, U251vIII cells were expanded in vitro until enough viable cells for implant were obtained. Cells were resuspended in DMEM without fetal bovine serum and mixed 1:1 with BD growth factor reduced matrigel (BD Biosciences, San Diego, Calif.) to reach a final concentration of 100×10⁶ cells/mL. CD-1 nu/nu mice were implanted with 100 μL of the cell/matrigel solution, or 10×10⁶ cells in the flank. Tumor volume was measured and on day eighteen the tumor volume ranged from 172 to 476 mm³ in 60 animals. These sixty animals were subsequently randomized into groups of ten animals each prior to treatment initiation. Treatment with the control conjugate, unconjugated 131 antibody, and Ab 131-DM1 was administered intravenously via the tail vein on day 18 post implantation.

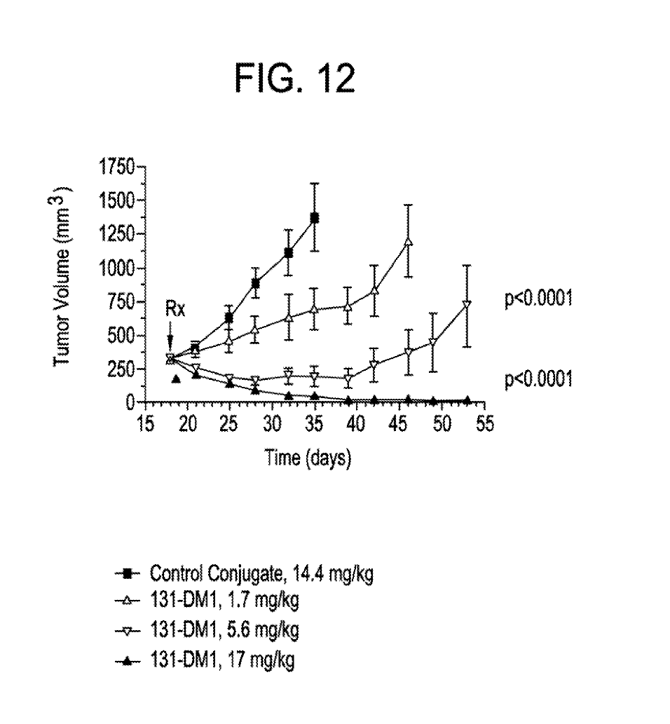

Animals received single IV doses of 14.4 mg/kg control conjugate in one cohort, 1.7 mg/kg Ab 131-DM1 in another cohort, 5.6 mg/kg Ab 131-DM1 in another cohort and 17 mg/kg Ab 131-DM1 in another cohort. Tumor volume was measured as described above twice weekly. As illustrated in FIG. 12, treatment with 5.6 or 17 mg/kg of Ab 131-DM1 led to a significant delay in tumor growth compared to Vehicle or control conjugate treated animals. Tumor regressions were observed at 5.6 and 17 mg/kg of Ab 131-DM1 with complete regressions at the 17 mg/kg dose. No effect on weight loss was observed in any animals

### Example 9B

### Ab 131-DM1 Conjugate in D317 Xenograft Model

D317 tumor fragments were serially passaged in CB-17/SCID mice. Animals bearing D317 human glioblastoma xenografts were euthanized and tumors removed under sterile conditions. Tumors were cut into similar sized fragments that fit into 13 gauge implant trocars. Tumors were implanted into the flanks of naïve CB-17/SCID mice. Tumor volume was measured post implantation and on day eleven the tumor volume ranged from 106 to 373 mm³ in 42 animals. Forty two animals were subsequently randomized into six groups of seven animals each for treatment initiation. Treatment was administered intravenously via the tail vein on days 11 and 18 post implantation. Animals received vehicle, anti-EGFRvIII antibody 131 at 20.5 mg/kg, Ab 131-DM1 conjugate at 4.9, 9.8 or 20.5 mg/kg (60, 120, and 250 μg DM1/kg, respectively) or control conjugate at 17.8 mg/kg (250 μg DM1/kg). On day 15 one animal in the vehicle group, two animals in the 131 antibody group, three animals in the control conjugate group, one animal in the 9.8 mg/kg dose group of Ab 131-DM1, and two animals in the 4.9 mg/kg dose group of Ab 131-DM1 were euthanized due to large tumor volume. On day 18 all remaining mice administered with vehicle, control conjugate, 131 antibody, and 4.9 mg/kg dose group of Ab 131-DM1 were euthanized due to large tumor volume. Two animals in the 9.8 mg/kg dose group of Ab 131-DM1 were euthanized due to large tumor size. On day 18 the four remaining mice in the 9.8 mg/kg dose group of AB 131-DM1 and all seven mice in the 20.5 mg/kg AB 131-DM1 group were dosed intravenously with a second dose. Mouse body weights and tumor volumes were monitored throughout the study which ended on day 29.

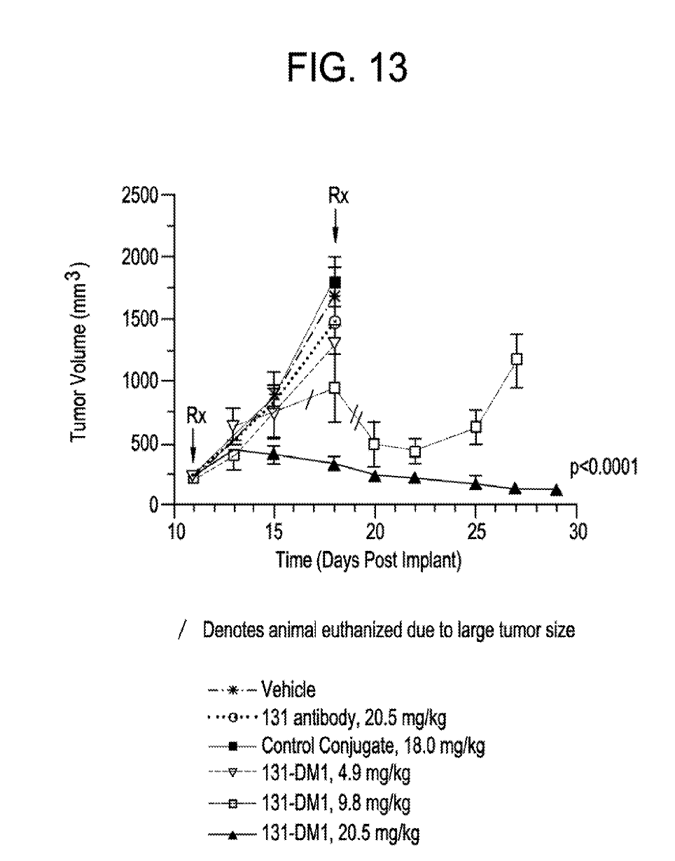

As illustrated in FIG. 13, treatment with 20.5 mg/kg of Ab 131-DM1 conjugate led to a significant delay in tumor growth compared to vehicle treated animals, whereas the 131 antibody and control conjugate had no effect.

### Example 9C

### Dose Response Experiment with Ab 131-DM1 Conjugate in a D317 Xenograft Model

D317 cells were resuspended in DMEM without fetal bovine serum and mixed 1:1 with BD growth factor reduced matrigel (BD Biosciences, San Diego, Calif.) to reach a final concentration of 1×10⁶ cells/mL. CB-17/SCID mice were implanted with 200 μL of the cell/matrigel solution, or 0.2×10⁶ cells in the flank. Day nine following implant tumor volume was measured, fifty animals with a range of 137-313 mm³ were randomized and treated. Mice received a single intravenous injection of either vehicle, control conjugate at 26.8 mg/kg (375 ug DM1/kg), or Ab 131-DM1 conjugate at 7.3, 14.6, or 22 mg/kg (125, 250, or 375 ug DM1/kg, respectively) on day nine post implantation.

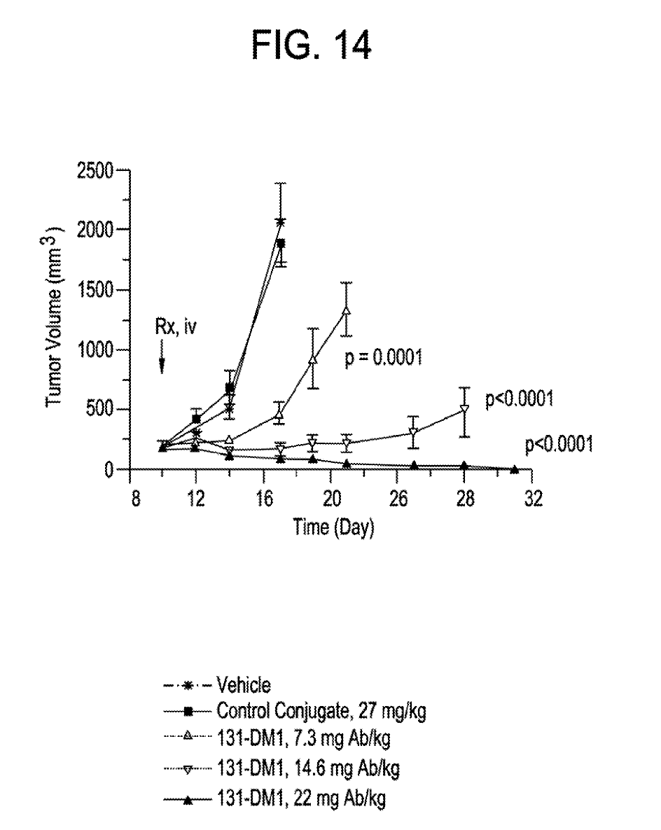

As illustrated in FIG. 14, treatment with Ab 131-DM1 at all doses led to a significant delay in tumor growth, with regression observed at 22 mg/kg. The control conjugate had no effect.

### Example 9D

### MRI Apparent Diffuission Coefficient in Ab 131-DM1 Conjugate Treated D317 Xenograft Model

In this study, the magnetic resonance imaging (MRI) apparent diffusion coefficient (ADC) was tested as an early readout of structural changes in tumor tissue as a result of Ab 131-DM1 treatment in an orthotopic model of glioblastoma.

CB17 SCID mice were implanted with D317 human glioblastoma cells (100,000 cells/mouse) via stereotactic injection into the right hemisphere of the brain at Day 0. At Day 7, mice were imaged with MRI and randomized into five treatment groups based on tumor volume. Mice were treated with vehicle, 6.5, 11, or 22 mg/kg Ab 131-DM1 i.v. twice per week, or temozolomide 10 mg/kg p.o. daily five days per week (N=8/group). Mice were subsequently imaged with MRI at Day 14 and Day 21. Tumor volumes in all mice were assessed by manually tracing hyperintense regions in multi-slice T2-weighted RARE (rapid acquisition with relaxation enhancement) images covering the entire tumor volume. The mean MRI apparent diffusion coefficient for each tumor in the vehicle and 22 mg/kg Ab 131-DM1-treated groups was calculated from diffusion-weighted spin echo images (b=100, 300, 700, 1000, and 1200 s/mm2), using manually traced regions over the entire tumor volume.

A dose-dependent effect of Ab 131-DM1 on tumor volume was observed at Day 21, though there was no significant difference in tumor volumes between the groups at Day 14. At Day 21, growth was inhibited in both the temozolomide and Ab 131-DM1 treated groups (22 and 11 mg/kg) relative to vehicle (p<0.0001). Mean MRI ADC values were significantly higher after treatment with Ab 131-DM1 (22 mg/kg) at both Day 14 (23%, p<0.01 vs vehicle) and Day 21 (32%, p<0.0001 vs vehicle), while no significant change occurred in the MRI ADC of the vehicle group at any timepoint.

Ab 131-DM1 shows dose-dependent growth inhibition of D317 cells orthotopically injected into the mouse brain. Increases in tumor apparent diffusion coefficient after Ab 131-DM1 treatment precede measurable inhibition of tumor growth, supporting MRI ADC as a early biomarker for therapeutic efficacy. The data also supports MRI ADC as an earlier biomarker for therapeutic efficacy than reduction in tumor volume.

### Example 10

### A Phase 1 First-in-Human Study Evaluating Safety, Tolerability, Pharmacokinetics and Pharmacodynamics of AB 131-DM1 in Subjects with Recurrent Malignant Glioma Expressing Mutant Epidermal Growth Factor Receptor Variant III (EGFRvIII

In an open-label, sequential dose exploration study of single agent Ab 131-DM1 is administered intravenously (IV) once every three weeks (Q3W) in subjects with recurrent GBM and/or AA. Eligible subjects enrolled in the study will receive Ab 131-DM1 IV Q3W as a 60 min infusion beginning at study day 1. Following the first two doses of Ab 131-DM1 and upon successful completion of a 28 day window for assessing dose limiting toxicities, subjects will undergo radiological assessment of their tumors with MRI during week 5. Dosing with Ab 131-DM1 (Q3W) may resume at week 7 unless there is for example, radiographic evidence of progressive disease (PD) per Macdonald criteria, the subject becomes intolerant to the study medication, or signs and symptoms of clinical progression are evident as determined by the principal investigatort. Subsequent tumor evaluations by MRI will occur at week 9 and every 8 weeks thereafter.

Enrollment will be restricted to patients showing evidence of EGFRvIII expression in tumor tissue. In addition, radiological assessment by MRI confirming measurable disease progression by the Macdonald criteria is also required for entry into the study.

An adaptive dose exploration will be used in the study (using a practical continual reassessment method [CRM] [Zhou, 2002]) and is aimed at determining the maximum tolerated dose (MTD), if feasible, and evaluating the safety, tolerability, PK and PD of AB 131-DM1. The MTD is defined as the maximum dose at which the probability of a doselimiting toxicity (DLT) is less than or equal to 25%.

To ensure a safe dose escalation, the maximum dose increase at any point will be ≦2× current dose. The pre-specified nominal doses for use in the dose exploration are 0.5, 1.0, 2.0, 3.0, 4.0 and 5.0 mg/kg of AB 131-DM1 (IV; Q3W). Intermediate doses (multiples of 0.5 mg/kg) and alternative dose frequencies may also be used if required.

Efficacy of the dosing regiman may be radiologically assessed using the Macdonald Criteria (See, Macdonald D R, Cascino T L, Schold S C Jr, Cairncross J G. Response criteria for phase II studies of supratentorial malignant glioma. J Clin Oncol. 1990; 8:1277-1280). or the Response Assessment in Neuro-Oncology (RANO) Criteria (See, Wen P Y, Macdonald D R, Reardon D A, et al. Updated Response Assessment Criteria for High-Grade Gliomas: Response Assessment in Neuro-Oncology Working Group. J ClinOncol. 2010; 28: 1963-1972).

### INCORPORATION BY REFERENCE

All references cited herein, including patents, patent applications, papers, text books, and the like, and the references cited therein, to the extent that they are not already, are hereby incorporated herein by reference in their entirety.

### EQUIVALENTS

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A method of treating a mammal having a tumor comprising administering an anti-EGFRvIII antibody-drug conjugate to the mammal in need thereof at a dose of about 0.1 mg/kg to about 10 mg/kg body weight.

2. The method of claim 1 wherein the anti-EGFRvIII antibody-drug conjugate is administered to the mammal in need thereof at a dose of at least 0.1, 0.5, 1.0, 1.5, 2.0, 2.5, 3.0, 3.5. 4.0, 4.5, 5.0, 5.5, 6.0, 6.5, 7.0, 7.5, 8.0 8.5, 9.0, or 9.5 mg/kg to no more than 11.0 mg/kg.

3. The method of claim 1 wherein the anti-EGFRvIII antibody-drug conjugate is administered to the mammal in need thereof at a dose of at least 0.5 to 1 mg/kg, 0.1 to 2 mg/kg, 2 to 3 mg/kg, 3 to 4 mg/kg, 4 to 5 mg/kg, 5 to 6 mg/kg, 6 to 7 mg/kg, 7 to 8 mg/kg, 8 to 9 mg/kg 9-10 mg/kg, 1.5 to 2.5 mg/kg, 2.5 to 3.5 mg/kg, 3.5 to 4.5 mg/kg, 4.5 to 5.5 mg/kg or 5.5 to 6.5 mg/kg.

4. The method of claim 1 wherein the administering step of the invention comprises administering the anti-EGFRvIII antibody-drug conjugate to the mammal intravenously, by bolus injection, intracerebrally or by sustained release.

5. The method claim 1 wherein the anti-EGFRvIII antibody-drug conjugate is administered at least twice every week, at least once every week, at least once every two weeks, at least once every three weeks or at least once every four weeks.

6. The method of claim 1 wherein the tumor in the mammal expresses EGFRvIII.

7. The method of claim 1 wherein the tumor in the mammal is a lung carcinoma, breast carcinoma, colon carcinoma, gastric carcinoma, renal carcinoma, head & neck carcinoma, prostate carcinoma, ovarian carcinoma, glioblastoma, an anaplastic astrocytoma, astrocytoma or a tumor comprising a glial component, particularly glioblastoma, anaplastic astrocytoma, astrocytoma or a tumor comprising a glial component.

8. The method of claim 7 wherein the tumor in the mammal is a glioblastoma or an anaplastic astrocytoma.

9. The method of claim 8 wherein the tumor in the mammal is recurrent glioblastoma or recurrent anaplastic astrocytoma.

10. The method of claim 1, wherein the tumor in the mammal is oligodenroglioma, oligoastrocytoma, gliosarcoma, mixed glioma, pilocytic astrocytoma, pleomorphic xanthoastrocytoma, subependymal giant cell astrocytoma, astroblastoma, spongioblastoma, gliomatosis cerebri, or neuronal-glial tumors including gangliglioma, or anaplastic ganglioglioma

11. The method of claim 1 wherein the mammal is alive more than 3, more than 4, more than 5 or more than 6 months after the first administration the anti-EGFRvIII antibody-drug conjugate

12. The method of claim 1, wherein the tumor in the mammal has not progressed after 3, after 4, after 5 or after 6 months from the first administration of the anti-EGFRvIII antibody-drug conjugate

13. The method of claim 1 wherein the mammal comprises a level of circulating tumor cells that is reduced as compared to the level of circulating tumor cells in the mammal before first administration of the anti-EGFRvIII antibody-drug conjugate.

14. The method of claim 1 wherein the mammal comprises a level of exosomes characteristic of a tumor which level is reduced as compared to the level of exosomes characteristic of a tumor in the mammal before the first administration of the anti-EGFRvIII antibody-drug conjugate.

15. The method of claim 1 wherein the tumor size in the mammal is reduced after administration of anti-EGFRvIII antibody-drug conjugate as compared to the tumor size prior to the first administration of anti-EGFRvIII antibody-drug conjugate.

16. The method claim 1 wherein the tumor size in the mammal is decreased at least 1%, at least 5%, at least 10%, at least 15%, at least 20%, at least 25%, at least 30%, at least 40%, at least 50%, at least 60%, at least 70%, at least 80%, at least 90% or 100% as compared to the tumor size in the mammal prior to first administration of the anti-EGFRvIII antibody-drug conjugate.

17. The method of claim 1 wherein, the mammal exhibits a complete or partial response.

18. The method of claim 1 wherein the mammal exhibits progression free survival of 6 months from the first administration of the anti-EGFRvIII antibody-drug conjugate.

19. The method of claim 15 as assessed by the Macdonald Criteria or RANO Criteria.

20. The method of claim 1 wherein the antibody of the anti-EGFRvIII antibody-drug conjugate is antibody 131.

21. The method of claim 1 wherein the, antibody of the anti-EGFRvIII antibody drug conjugate comprises a heavy chain variable region comprising the amino acid sequence of SEQ ID NO 2 and a light chain variable region comprising an amino acid sequence of SEQ ID NO 19.

22. The method of claim 1 wherein the drug conjugated to the anti-EGFRvIII antibody is a radioactive isotope, a chemotherapeutic agent, a toxin or fragment or variants thereof.

23. The method of claim 1 wherein the anti-EGFRvIII antibody is conjugated to at least one to 10, at least one to 5, at least 3-4 or at least 3-5 maytansinoid DM1 molecules.

24. The method of claim 1 wherein the drug is conjugated to the anti-EGFRvIII antibody via a non-cleavable linker group.

25. The method of claim 1 wherein the drug is conjugated to the anti-EGFRvIII antibody via succinimidyl-4-(N-maleimidomethyl)cyclohexane-1-carboxylate (SMCC).

26. The method of claim 1 wherein anti-EGFRvIII antibody-drug conjugate is Ab 131-DM1 depicted in FIGS. 8A, 8B and 8D and comprises the full length heavy chain depicted in FIG. 8B and the full length light chain depicted in FIG. 8D.

27. The method of claim 1 wherein the anti-EGFRvIII antibody comprising heavy chain variable region comprising the amino acid sequence of SEQ ID NO 2 and a light chain variable region comprising an amino acid sequence of SEQ ID NO 19 and the anti-EGFRvIII antibody is conjugated to 3-5 maytansinoids by a MCC linker.

28. The method of claim 1 wherein the administration step is carried out prior to, in combination with or after treating the mammal by applying surgery, applying radiationtherapy, applying whole brain radiation therapy in the primary setting, applying focal radiation therapy in the recurrent setting, administering temozolomide in the primary and recurrent setting, administering bevacizumab, administering irinotecan, administering PCV, procabazine, lomustine [CCNU], vincristine, implanting a Gliadel wafer (polifeprosan impregnated with BCNU), administering a tyrosine kinase inhibitor, administering a radio-sensitizing agent, administering a vaccine based therapy, administering an antibody drug conjugate or administering a Bi-specific T-cell enhancer in the primary or recurrent settings. or administering a targeted drug to the mammal.

29. The method claim 1 wherein the mammal is human.


SEQUENCE LISTING (144 sequences)

SEQ ID NO: 1 - protein, 109 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARWGQGTLVTVSS

SEQ ID NO: 2 - protein, 124 aa, Homo sapiens
QVQLVESGGGVVQSGRSLRLSCAASGFTFRNYGMHWVRQAPGKGLEWVAVIWYDGSDKYYADSVRGRFTISRDNSKNTLYLQMNSLRAEDTAVYYCARDGYDILTGNPRDFDYWGQGTLVTVSS

SEQ ID NO: 3 - protein, 112 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARWGQGTLVTVSS

SEQ ID NO: 4 - protein, 118 aa, Homo sapiens
QVQLQQSGPGLVKPSQTLSLTCAISGDSVSSNNAAWNWIRQSPARGLEWLGRTYYRSKWYNDYVVSVKSRITINPDTSKNQFSLQLNSVTPEDTAVYYCVRATAFDYWGQGTLVTVSS

SEQ ID NO: 5 - protein, 118 aa, Homo sapiens
QVQLQQSGPGLVKPSQTLSLTCAISGDSVSSYSSAWNWIRQSPSRGLEWLGRAYHRSRWYYEYAVSVKSRINITPDTSKNQFSLQLNSVTPEDTAVYYCARGSRFDYWGQGTLVTVSS

SEQ ID NO: 6 - protein, 110 aa, Homo sapiens
QVQLQESGPFLVKPSQTLSLTCTVSGGSISSGGYYWSWIRQHPGKGLEWIGYIYYSGSTYYNPSLKSRVTISVDTSKNQFSLKLSSVTAADTAVYYCARWGQGTLVTVSS

SEQ ID NO: 7 - protein, 126 aa, Homo sapiens
QVQLQESGPFLVKPSQTLSLTCTVSGGSISSGGYYWSWIRQHPGKGLEWIGFIYYRGNTYYNPSLKSRVTISVDTSKNQFSLKLSSVTAADTAVYYCARDGYCSRTGCYGGWFDPWGQGTLVTVSP

SEQ ID NO: 8 - protein, 109 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGQGTLVTVSS

SEQ ID NO: 9 - protein, 116 aa, Homo sapiens
EGQLLESGGGWVQPGESLRLSCAASGFTFSSYAMSWVRQAPGKGLEWVSAISGSGGSTNYADSVKGRFTISRDNSKNTLYLQVNSLRVEDTAVYYCAGSSGWSEYWGQGTLVTVSS

SEQ ID NO: 10 - protein, 116 aa, Homo sapiens
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSS

SEQ ID NO: 11 - protein, 109 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKWGQGTTVTVSS

SEQ ID NO: 12 - protein, 128 aa, Homo sapiens
QVQLVESGGGVVQPGRSLRLSCVASGFTLSSYGMHWVRQAPGKGLEWVAVTSYDGSKKDYADSAKGRFTISRDNSKNTLYLQMNSLRAEDTAVYYCVSEGYCSSSSCYKYYYYGMDVWGQGTTVTVSS

SEQ ID NO: 13 - protein, 128 aa, Homo sapiens
QVQLVESGGGVVQPGRSLRLSCAASGFTLSSYGMHWVRQAPGKGLEWVAVMSYDGSKEDYADSVKGRFTISRDNSENTLYLQMNSLRAEDTAVYYCVSEGYCSSRSCYKYYYYGMDVWGQGTTVTVSS

SEQ ID NO: 14 - protein, 109 aa, Homo sapiens; identical to SEQ ID NO: 11

SEQ ID NO: 15 - protein, 128 aa, Homo sapiens
QVQLVESGGGVVQPGRSLRLSCVASGFTLSSYGMHWVRQAPGKGLEWVAVTSYDGSKKDYADSVKGRFTISRDNSKNTLYLQMNSLRAEDTAVYYCVSEGYCDSSSCYKYYYYGMDVWGQGTTVTVSS

SEQ ID NO: 16 - protein, 128 aa, Homo sapiens
QVQLVESGGGVVQPGRSLRLSCVASGFTLSSYGMHWVRQAPGKGLEWVAVTSYDGSKKDYADSVKGRFTISRDNSKNTLYLQMNSLRAEDTAVYYCVSEGYCDSTSCYKYYYYGMDVWGQGTTVTVSS

SEQ ID NO: 17 - protein, 128 aa, Homo sapiens
QVQLVESGGGVVQPGRSLRLSCVASGFTLSSYGMHWVRQALGKGLEWVAVTSYDGSKKDYADSVKGRFTISRDNSKNTLYLQMNSLRAEDTAVYYCVSEGYCDSTSCYKYYYYGMDVWGQGTTVTVSS

SEQ ID NO: 18 - protein, 112 aa, Homo sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPWTFGQGTKVEIK

SEQ ID NO: 19 - protein, 112 aa, Homo sapiens
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGQGTKVEIK

SEQ ID NO: 20 - protein, 112 aa, Homo sapiens
AIVLTQTPLSSPVTLGQPASISCRSSQSLVHRDGNTYLSWLQQRPGQPPRLLIYKISNRFSGVPDRFSGSGAGTDFTLKISRVEPDDVGVYYCMHTTQLPWTFGQGTKVEIK

SEQ ID NO: 21 - protein, 112 aa, Homo sapiens
DIVMTQTPLSSPVTLGQPASISCRSSQSLVHRDGNTYLSWLQQRPGQPPRLLIYKISNRFSGVPDRFSGSGAGTDFTLKISRVEAEDVGIYFCMHTTQFPWTFGQGTRVEIK

SEQ ID NO: 22 - protein, 112 aa, Homo sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPITFGQGTRLEIK

SEQ ID NO: 23 - protein, 112 aa, Homo sapiens
DIVMTQTPLSSPVTLGQPASISCRSSQSLIHTDGNIYLSWLQQRPGQPPRLLIYKISNRFSGVPDRFSGSGAGTDFTLKISRVEAEDVGVYYCMQGTQFPITFGQGTRLEIK

SEQ ID NO: 24 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPLTFGGGTKVEIK

SEQ ID NO: 25 - protein, 107 aa, Homo sapiens
DIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIK

SEQ ID NO: 26 - protein, 107 aa, Homo sapiens
DIQMTQSPSSLSASVGDSVTITCRTSQGIRKNLGWYQQKPGKAPKRLIYAASSLQSGVPSRFSGSGSGTEFTLTISRLQPEDFATYYCLQHHSYPLTFGGGTRVEIR

SEQ ID NO: 27 - protein, 111 aa, Homo sapiens
DIVMTQSPLLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKVEIK

SEQ ID NO: 28 - protein, 112 aa, Homo sapiens
DIVMTQSPLSLPVTPGEPASISCRSSQSLLYRNGNNYLDWYLQRPGQSPQLLIYLGSNRASGVPDRFSGSGSGTDFTLKISRVEAEDVGVYYCMQALQTPRTFGQGTKVEIK

SEQ ID NO: 29 - protein, 112 aa, Homo sapiens
DIVMTQSPLSLPVTPGEPASISCRSSQSLLYRNGNNYLDWYLQKPGQSPQLLIYLGSNRASGVPDRFSGSGSGTDFTLNISRVEAEDVGHYYCMQALQTPRTFGQGTKVEIK

SEQ ID NO: 30 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKVEIK

SEQ ID NO: 31 - protein, 112 aa, Homo sapiens; identical to SEQ ID NO: 28

SEQ ID NO: 32 - protein, 112 aa, Homo sapiens; identical to SEQ ID NO: 28

SEQ ID NO: 33 - protein, 112 aa, Homo sapiens; identical to SEQ ID NO: 28

SEQ ID NO: 34 - DNA, 336 bp, Homo sapiens
gatattgtgatgacccagactccactctcctcacctgtcacccttggacagccggcctccatctcctgcaggtctagtcaaagcctcatacacactgatggaaacatctatttgagttggcttcagcagaggccaggccagcctccaagactcctaatttataagatttctaatcggttctctggggtcccagacagattcagtggcagtggggcagggacagatttcacactgaagatcagcagggtggaagctgaggatgtgggggtttattactgcatgcaaggtacacaatttcctatcaccttcggccaagggacacgactggagattaaa

SEQ ID NO: 35 - DNA, 375 bp, Homo sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggttcatctattacagagggaacacctactacaacccgtccctcaagagtcgagttaccatatcagttgacacgtctaagaaccagttctccctgaagctgagctctgtgactgccgcggacacggccgtgtattactgtgcgcgagacggatattgtagtagaaccggctgctatggcggctggttcgacccctggggccagggaaccctggtcacgtctcct

SEQ ID NO: 36 - DNA, 335 bp, Homo sapiens
atattgtgatgactcagtctccactctccctgcccgtcacccctggagagccggcctccatctcctgcaggtctagtcagagcctcctgtatagaaatggaaacaactatttggattggtatctgcagaagccagggcagtctccacagctcctgatctatttgggttctaatcgggcctccggggtccctgacaggttcagtggcagtggatcgggcacagattttacactgaacatcagcagagtggaggctgaggatgttgggcattattactgcatgcaggctctacaaactcctcggacgttcggccaagggaccaaggtggaaatcaaa

SEQ ID NO: 37 - DNA, 384 bp, Homo sapiens
caggtgcagctggtggagtctgggggaggcgtggtccagcctgggaggtccctgagactctcctgtgcagcctccggattcaccctcagtagctatggcatgcactgggtccgccaggctccaggcaaggggctggagtgggtggcagttatgtcatatgatggaagtaaagaagactatgcagactccgtgaagggccgattcaccatctctagagacaattccgagaacatgctgtatctgcaaatgaacagcctgagagctgaggacacggctgtatattactgtgtgagcgaaggatattgtagtagtcgtagctgctataagtactactactacggcatggacgtctggggccaagggaccacggtcaccgtctcctca

SEQ ID NO: 38 - DNA, 336 bp, Homo sapiens
gatactgtgatgacccagactccactctcctcacatgtaacccttggacagccggcctccatctcctgcaggtctagtcaaagcctcgtacacagtgatggaaacacctacttgagttggcttcagcagaggccaggccaacctccaagactcctaatttataggatttctaggcggttctctggggtcccagacagattcagtggcagtggggcagggacagatttcacactggaaatcagcagggtggaggctgaggatgtcggggtttattactgcatgcaatctacacacgttcctcggacgttcggccaagggaccaaggtggagatcaaa

SEQ ID NO: 39 - DNA, 372 bp, Homo sapiens
caggtgcagctggtggagtctgggggaggcgtggtccagtctgggaggtccctgagactctcctgtgcagcgtctggattcaccttcagaaactatggcatgcactgggtccgccaggctccaggcaaggggctggagtgggtggcagttatatggtatgatggaagtgataaatactatgcagactccgtgaggggccgattcaccatctccagagacaattccaagaacacgctgtatctgcaaatgaacagcctgagagccgaggacacggctgtgtattactgtgcgagagatggctacgatattttgactggtaatcctagggactttgactactggggccagggaaccctggtcaccgtctcctca

SEQ ID NO: 40 - DNA, 348 bp, Homo sapiens
gaggtgcaggtgttggagtctgggggaggcttggtacagcctggggggtccctgagactctcctgtgcagcctctggattcacctttagcagctatgccatgagctgggtccgccaggctccagggaaggggctggagtgggtctcggctattagtggtagtggtggtagtacaaactacgcagactccgtgaagggccggttcaccatctccagagacaattccaagaacacactgtatctgcaaatgaacagcctgagagccgaggacacggccgtctattactgtgctgggagcagtggctggtccgagtactggggccagggaaccctggtcaccgtctcctcg

SEQ ID NO: 41 - DNA, 321 bp, Homo sapiens
gacatccagatgacccagtctccatcctccctgtctgcatctgtaggagacagagtcaccatcacttgccgggctagtcagggcattagaaataatttagcctggtatcagcagaaaccagggaaagcccctaagcgcctgatctatgctgcctccaatttgcaaagtggggtcccatcaaggttcaccggcagtggatctgggacagaattcactctcatagtcagcagcctgcagcctgaagattttgcgacttattactgtctacagcatcacagttacccgctcacttccggcggagggaccaaggtggagatcaaa

SEQ ID NO: 42 - DNA, 336 bp, Homo sapiens
gatattgtgatgacccagactccactctcctcacctgtcacccttggacagccggcctccatctcctgcaggtctagtcaaagcctcgtacacagggatggaaatacctacttgagttggcttcagcagaggccaggccagcctccaagactcctaatttataagatttctaaccggttctctggggtcccagacagattcagtggcagtggggcagggacagatttcacactgaaaattagcagggtggaagctgaggatgtcgggatttatttctgcatgcatactacacaatttccttggacgttcggccaagggaccagggtggaaatcaaa

SEQ ID NO: 43 - DNA, 354 bp, Homo sapiens
caggtacagctgcagcagtcaggtccaggactggtgaagccctcgcagaccctctcactcacctgtgccatctccggggacagtgtctctagctacagttctgcttggaactggatcaggcagtccccatcgagaggccttgagtggctgggaagggcatatcacaggtccaggtggtattacgagtatgcagtatcggtgaaaagtcgaataaacatcaccccagacacatccaagaaccagttctccctgcagctgaactctgtgactcccgaggacacggctgtgtattactgtgcaagaggcagtcgctttgactactggggccagggaaccctggtcaccgtctcctca

SEQ ID NO: 44 - DNA, 354 bp, Homo sapiens
caggtacagctgcagcagtcaggtccaggactggtgaagccctcgcagaccctctcactcacctgtgccatctccggggacagtgtctctagcaacaatgctgcttggaactggatcaggcagtccccagcgagaggccttgagtggctgggaaggacatactacaggtccaagtggtataatgattatgtagtatctgtgaaaagtcgaataaccatcaacccagacacatccaagaaccagttctccctgcagctgaactctgtgactcccgaggacacggctgtgtattactgtgtaagaggcagtcgctttgactactggggccagggaaccctggtcaccgtctcctca

SEQ ID NO: 45 - DNA, 336 bp, Homo sapiens
gctattgtgttgacccagactccactctcctcacctgtcacccttggacagccggcctccatctcctgcaggtctagtcaaagcctcgttcacagggatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaacctgacgatgtcggggtttattactgcatgcatactacacaacttccttggacgttcggccaagggaccaaggtggaaatcaaa

SEQ ID NO: 46 - DNA, 336 bp, Homo sapiens
gatattgtgatgactcagtctccactctccctgcccgtcacccctggagagccggcctccatctcctgcaggtctagtcagagcctcctatatagaaatggaaacaactatttggattggtatctgcagaggccagggcagtctccacaactcctgatctatttgggttctaatcgggcctccggggtccctgacaggttcagtggcagtggatcaggcacagattttacattgaaaatcggcagagtggaggctgaggatgttggggtttattactgcatgcaggctctacaaactcctcggacgttcggccaagggaccaaggtggaaatcaaa

SEQ ID NO: 47 - DNA, 384 bp, Homo sapiens
caggtgcagctggtggagtctgggggaggcgtggtccagcctgggaggtccctgagactctcctgtgtagcctctggattcaccctcagtagctatggcatgcactgggtccgccaggctccaggcaaggggctggagtgggtggcagtgacatcatatgatggaagtaaaaaagactatgcagactccgcgaagggccgattcaccatctccagagacaattccaagaacacgctgtatctgcaaatgaacagcctgagagctgaggacacggctgtgtattactgtgtgagcgaaggatattgtagtagtagtagctgctataagtactactattacggtatggacgtctggggccaagggaccacggtcaccgtctcttca

SEQ ID NO: 48 - DNA, 348 bp, Homo sapiens
gaggggcagctgttggagtctgggggaggctgggtacagcctggggagtccctgagactctcctgtgcagcctctggattcacctttagcagctatgccatgagctgggtccgccaggctccagggaaggggctggagtgggtctcggctattagtggtagtggtggtagcacaaattacgcagactccgtgaagggccggttcaccatctccagagacaattccaagaacacgctgtatctgcaagtgaacagcctgagagtcgaggacacggccgtatattactgtgctgggagcagtggctggtccgagtactggggccagggaaccctggtcaccgtctcctca

SEQ ID NO: 49 - DNA, 321 bp, Homo sapiens
gacatccagatgacccagtctccatcctccctgtctgcatctgtaggagacagcgtcaccatcacttgccggacaagtcagggcattagaaaaaatttaggctggtatcagcagaaaccagggaaagcccctaagcgcctgatctatgctgcatccagtttacaaagtggggtcccatcaaggttcagcggcagtggatctgggacagaattcactctcacaatccgcagcctgcagcctgaagattttgcaacttattactgtctccagcatcatagttacccgctcactttcggcggagggaccagggtggagatcaga

SEQ ID NO: 50 - DNA, 336 bp, Homo sapiens
gatattgtgatgactcagtctccactctccctgcccgtcacccctggagagccggcctccatctcctgcaggtctagtcagagcctcctatatagaaatggaaacaactatttggattggtatctgcagaggccagggcagtctccacaactcctgatctatttgggttctaatcgggcctccggggtccctgacaggttcagtggcagtggatcaggcacagattttacactgaaaatcagcagagtggaggctgaggatgttggggtttattactgcatgcaggctctacaaactcctcggacgttcggccaagggaccaaggtggaaatcaaa

SEQ ID NO: 51 - DNA, 384 bp, Homo sapiens
caggtgcagctggtggagtctgggggaggcgtggtccagcctgggaggtccctgagactctcctgtgtagcctctggattcaccctcagtagctatggcatgcactgggtccgccaggctccaggcaaggggctggagtgggtggcagtgacatcatatgatggaagtaaaaaagactatgcagactccgtgaagggccgattcaccatctccagagacaattccaagaacacgctgtatctgcaaatgaacagcctgagagctgaggacacggctgtgtattactgtgtgagcgaaggatattgtgatagtagtagctgctataagtactactactacggtatggacgtctggggccaagggaccacggtcaccgtctcttca

SEQ ID NO: 52 - DNA, 336 bp, Homo sapiens; identical to SEQ ID NO: 50

SEQ ID NO: 53 - DNA, 384 bp, Homo sapiens
caggtgcagctggtggagtctgggggaggcgtggtccagcctgggaggtccctgagactctcctgtgtagcctctggattcaccctcagtagctatggcatgcactgggtccgccaggctctaggcaaggggctggagtgggtggcagtgacatcatatgatggaagtaaaaaagactatgcagactccgtgaagggccgattcaccatctccagagacaattccaagaacacgctgtatctgcaaatgaacagcctgagagctgaggacacggctgtgtattactgtgtgagcgaaggatattgtgatagtactagttgctataagtactactactacggtatggacgtctggggccaagggaccacggtcaccgtctcttca

SEQ ID NO: 54 - DNA, 336 bp, Homo sapiens; identical to SEQ ID NO: 50

SEQ ID NO: 55 - DNA, 384 bp, Homo sapiens
caggtgcagctggtggagtctgggggaggcgtggtccagcctgggaggtccctgagactctcctgtgtagcctctggattcaccctcagtagctatggcatgcactgggtccgccaggctccaggcaaggggctggagtgggtggcagtgacatcatatgatggaagtaaaaaagactatgcagactccgtgaagggccgattcaccatctccagagacaattccaagaacacgctgtatctgcaaatgaacagcctgagagctgaggacacggctgtgtattactgtgtgagcgaaggatattgtgatagtactagctgctataagtactactactacggtatggacgtctggggccaagggaccacggtcaccgtctcttca

SEQ ID NO: 56 - protein, 14 aa, Artificial Sequence
  Synthetic peptide sequence
LEEKKGNYVVTDHC

SEQ ID NO: 57 - protein, 10 aa, Artificial Sequence
  Synthetic peptide sequence
EEKKGNYVVT

SEQ ID NO: 58 - protein, 5 aa, Artificial Sequence
  Synthetic peptide sequence
LEEKK

SEQ ID NO: 59 - protein, 12 aa, Artificial Sequence
  Synthetic peptide sequence
LEEKKGNYVVTD

SEQ ID NO: 60 - protein, 4 aa, Artificial Sequence
  Synthetic peptide sequence
EKNY

SEQ ID NO: 61 - protein, 5 aa, Artificial Sequence
  Synthetic peptide sequence
EEKGN

SEQ ID NO: 62 - DNA, 35 bp, Artificial Sequence
  Synthetic oligonucleotide primer sequence
ggatctcgagccagaccggaacgacaggccacctc

SEQ ID NO: 63 - DNA, 34 bp, Artificial Sequence
  Synthetic oligonucleotide primer sequence
cggatctcgagccggagcccagcactttgatctt

SEQ ID NO: 64 - DNA, 35 bp, Artificial Sequence
  Synthetic oligonucleotide primer sequence
cggatgaattcccagaccggacgacaggccacctc

SEQ ID NO: 65 - DNA, 20 bp, Artificial Sequence
  Synthetic oligonucleotide primer sequence
ctttcttttcctccagagcc

SEQ ID NO: 66 - DNA, 21 bp, Artificial Sequence
  Synthetic oligonucleotide primer sequence
gtaattatgtggtgacagatc

SEQ ID NO: 67 - DNA, 35 bp, Artificial Sequence
  Synthetic oligonucleotide primer sequence
cggatctcgagctcaagagagcttggttgggagct

SEQ ID NO: 68 - DNA, 29 bp, Artificial Sequence
  Synthetic oligonucleotide primer sequence
ggtggcggtacctggacaagaccgttgcg

SEQ ID NO: 69 - DNA, 37 bp, Artificial Sequence
  Synthetic oligonucleotide primer sequence
ataagaatgcggccgctcatttacccggagagcggga

SEQ ID NO: 70 - DNA, 32 bp, Artificial Sequence
  Synthetic oligonucleotide primer sequence
ctactagctagccaccatgcgaccctccggga

SEQ ID NO: 71 - DNA, 26 bp, Artificial Sequence
  Synthetic oligonucleotide primer sequence
cggggtacccggcgatggacgggatc

SEQ ID NO: 72 - protein, 12 aa, Artificial Sequence
  Synthetic peptide sequence
ALEEKKGNYVVT

SEQ ID NO: 73 - protein, 12 aa, Artificial Sequence
  Synthetic peptide sequence
EEKKGNYVVTDH

SEQ ID NO: 74 - protein, 12 aa, Artificial Sequence
  Synthetic peptide sequence
EKKGNYVVTDHG

SEQ ID NO: 75 - protein, 12 aa, Artificial Sequence
  Synthetic peptide sequence
KKGNYVVTDHGS

SEQ ID NO: 76 - protein, 12 aa, Artificial Sequence
  Synthetic peptide sequence
KGNYVVTDHGSC

SEQ ID NO: 77 - protein, 12 aa, Artificial Sequence
  Synthetic peptide sequence
GNYVVTDHGSCV

SEQ ID NO: 78 - protein, 12 aa, Artificial Sequence
  Synthetic peptide sequence
NYVVTDHGSCVR

SEQ ID NO: 79 - protein, 12 aa, Artificial Sequence
  Synthetic peptide sequence
YVVTDHGSCVRA

SEQ ID NO: 80 - protein, 12 aa, Artificial Sequence
  Synthetic peptide sequence
AEEKKGNYVVTD

SEQ ID NO: 81 - protein, 12 aa, Artificial Sequence
  Synthetic peptide sequence
LAEKKGNYVVTD

SEQ ID NO: 82 - protein, 12 aa, Artificial Sequence
  Synthetic peptide sequence
LEAKKGNYVVTD

SEQ ID NO: 83 - protein, 12 aa, Artificial Sequence
  Synthetic peptide sequence
LEEAKGNYVVTD

SEQ ID NO: 84 - protein, 12 aa, Artificial Sequence
  Synthetic peptide sequence
LEEKAGNYVVTD

SEQ ID NO: 85 - protein, 12 aa, Artificial Sequence
  Synthetic peptide sequence
LEEKKANYVVTD

SEQ ID NO: 86 - protein, 12 aa, Artificial Sequence
  Synthetic peptide sequence
LEEKKGAYVVTD

SEQ ID NO: 87 - protein, 12 aa, Artificial Sequence
  Synthetic peptide sequence
LEEKKGNAVVTD

SEQ ID NO: 88 - protein, 12 aa, Artificial Sequence
  Synthetic peptide sequence
LEEKKGNYAVTD

SEQ ID NO: 89 - protein, 12 aa, Artificial Sequence
  Synthetic peptide sequence
LEEKKGNYVATD

SEQ ID NO: 90 - protein, 12 aa, Artificial Sequence
  Synthetic peptide sequence
LEEKKGNYVVAD

SEQ ID NO: 91 - protein, 12 aa, Artificial Sequence
  Synthetic peptide sequence
LEEKKGNYVVTA

SEQ ID NO: 92 - protein, 22 aa, Artificial Sequence
  Synthetic peptide sequence
ATCVKKCPRNYVVTDHGSCVRA

SEQ ID NO: 93 - protein, 19 aa, Artificial Sequence
  Synthetic peptide sequence
LEEKKGNYVVTDHGSCVRA

SEQ ID NO: 94 - protein, 10 aa, Artificial Sequence
  Synthetic peptide sequence
EEKKGNYVVT

SEQ ID NO: 95 - protein, 12 aa, Artificial Sequence
  Synthetic peptide sequence
LEEKKGNYVVTD

SEQ ID NO: 96 - protein, 6 aa, Artificial Sequence
  Synthetic peptide sequence
YVVTDH

SEQ ID NO: 97 - protein, 5 aa, Artificial Sequence
  Synthetic peptide sequence
YVVTD

SEQ ID NO: 98 - protein, 10 aa, Artificial Sequence
  Synthetic peptide sequence
EEKKGNYVVT

SEQ ID NO: 99 - protein, 6 aa, Artificial Sequence
  Synthetic peptide sequence
GNYVVT

SEQ ID NO: 100 - protein, 23 aa, Artificial Sequence
  Synthetic peptide sequence
DTVMTQTPLSSHVTLGQPASISC

SEQ ID NO: 101 - protein, 16 aa, Artificial Sequence
  Synthetic peptide sequence
RSSQSLVHSDGNTYLS

SEQ ID NO: 102 - protein, 14 aa, Artificial Sequence
  Synthetic peptide sequence
WLQQRPGPPRLLIY

SEQ ID NO: 103 - protein, 7 aa, Artificial Sequence
  Synthetic peptide sequence
RISRRFS

SEQ ID NO: 104 - protein, 32 aa, Artificial Sequence
  Synthetic peptide sequence
GVPDRFSGSGAGTDFTLEISRVEAEDVGVYYC

SEQ ID NO: 105 - protein, 9 aa, Artificial Sequence
  Synthetic peptide sequence
MQSTHVPWT

SEQ ID NO: 106 - protein, 9 aa, Artificial Sequence
  Synthetic peptide sequence
FGQTKVEIK

SEQ ID NO: 107 - protein, 30 aa, Artificial Sequence
  Synthetic peptide sequence
QVQLVESGGGVVQSGRSLRLSCAASGFTFR

SEQ ID NO: 108 - protein, 5 aa, Artificial Sequence
  Synthetic peptide sequence
NYGMH

SEQ ID NO: 109 - protein, 14 aa, Artificial Sequence
  Synthetic peptide sequence
WVRQAPGKGLEWVA

SEQ ID NO: 110 - protein, 17 aa, Artificial Sequence
  Synthetic peptide sequence
VIWYDGSDKYYADSVRG

SEQ ID NO: 111 - protein, 32 aa, Artificial Sequence
  Synthetic peptide sequence
RFTISRDNSKNTLYLQMNSLRAEDTAVYYCAR

SEQ ID NO: 112 - protein, 15 aa, Artificial Sequence
  Synthetic peptide sequence
DGYDILTGNPRDFDY

SEQ ID NO: 113 - protein, 11 aa, Artificial Sequence
  Synthetic peptide sequence
WGQGTLVTVSS

SEQ ID NO: 114 - protein, 23 aa, Artificial Sequence
  Synthetic peptide sequence
DIVMTQTPLSSPVTLGQPASISC

SEQ ID NO: 115 - protein, 15 aa, Artificial Sequence
  Synthetic peptide sequence
WLHQRPGQPPRLLIY

SEQ ID NO: 116 - protein, 7 aa, Artificial Sequence
  Synthetic peptide sequence
KISNRFS

SEQ ID NO: 117 - protein, 32 aa, Artificial Sequence
  Synthetic peptide sequence
GVPDRFSGSGAGTAFTLKISRVEAEDVGVYYC

SEQ ID NO: 118 - protein, 9 aa, Artificial Sequence
  Synthetic peptide sequence
MQATQLPRT

SEQ ID NO: 119 - protein, 11 aa, Artificial Sequence
  Synthetic peptide sequence
FGQGTKVEIKR

SEQ ID NO: 120 - protein, 30 aa, Artificial Sequence
  Synthetic peptide sequence
QVQLVESGGGVVQPGRSLRLSCAASGFTFS

SEQ ID NO: 121 - protein, 5 aa, Artificial Sequence
  Synthetic peptide sequence
SYGMH

SEQ ID NO: 122 - protein, 14 aa, Artificial Sequence
  Synthetic peptide sequence
WVRQAPGKGLEWVA

SEQ ID NO: 123 - protein, 17 aa, Artificial Sequence
  Synthetic peptide sequence
VIWYDGSNKYYVDSVKG

SEQ ID NO: 124 - protein, 32 aa, Artificial Sequence; identical to SEQ ID NO: 111
  Synthetic peptide sequence

SEQ ID NO: 125 - protein, 11 aa, Artificial Sequence
  Synthetic peptide sequence
DGWQQLAPFDY

SEQ ID NO: 126 - protein, 12 aa, Artificial Sequence
  Synthetic peptide sequence
WGQGTLVTVSSA

SEQ ID NO: 127 - protein, 6 aa, Artificial Sequence
  Synthetic peptide sequence
EEKKGN

SEQ ID NO: 128 - DNA, 33 bp, Artificial Sequence
  Synthetic peptide sequence
ataaaagcttctggaggaaaagaaaggtaatta

SEQ ID NO: 129 - protein, 33 aa, Artificial Sequence
  Synthetic peptide sequence
TTATTGGTACCTCAGGCGATGGACGGGATCTTA

SEQ ID NO: 130 - protein, 14 aa, Artificial Sequence
  Synthetic peptide sequence
LEEKKGNYVVTDHG

SEQ ID NO: 131 - protein, 7 aa, Artificial Sequence
  Synthetic peptide sequence
EEKKGNY

SEQ ID NO: 132 - protein, 11 aa, Artificial Sequence
  Synthetic peptide sequence
LEEKKGNYVVT

SEQ ID NO: 133 - protein, 8 aa, Artificial Sequence
  Synthetic peptide sequence
LEEKKGNY

SEQ ID NO: 134 - protein, 1186 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 135 - protein, 919 aa, Homo sapiens
LEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 136 - protein, 268 aa, Homo sapiens
VCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPR

SEQ ID NO: 137 - DNA, 512 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgtagactccgtgaagggccgattcaccatctccagagacaattccaagaacacgctgtatctgcaaatgaacagcctgagagccgaggacacggctgtgtattactgtgcgagagatggatggcagcagctggccccctttgactactggggccagggaaccctggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgca

SEQ ID NO: 138 - protein, 170 aa, Homo sapiens
QVQLVESGGGVVQPGRSLRLSCAASGFTFSSYGMHWVRQAPGKGLEWVAVIWYDGSNKYYVDSVKGRFTISRDNSKNTLYLQMNSLRAEDTAVYYCARDGWQQLAPFDYWGQGTLVTVSSASTKGPSVFPLAPSSKSTSGGTAALGCLVKDYFPEPVTVSWNSGALTSGV

SEQ ID NO: 139 - DNA, 496 bp, Homo sapiens
gatattgtgatgacccagactccactctcctcacctgtcacccttggacagccggcctccatctcctgcaggtctagtcaaagcctcgtgcatagtgatggaaacacctacttgagttggcttcaccagaggccaggccagcctccaagactcctaatttataagatttctaaccggttctctggggtcccagacagattcagtggcagtggggcagggacagctttcacactgaaaatcagcagggtggaagctgaggatgtcggggtttattactgcatgcaagctacacaacttcctcggacgttcggccaagggaccaaggtggaaatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccagg

SEQ ID NO: 140 - protein, 165 aa, Homo sapiens
DIVMTQTPLSSPVTLGQPASISCRSSQSLVHSDGNTYLSWLHQRPGQPPRLLIYKISNRFSGVPDRFSGSGAGTAFTLKISRVEAEDVGVYYCMQATQLPRTFGQGTKVEIKRTVAAPSVFIFPPSDEQLKSGTASVVCLLNNFYPREAKVQWKVDNALQSGNSQ

SEQ ID NO: 141 - protein, 110 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARWGQGTLVTVSSA

SEQ ID NO: 142 - protein, 121 aa, Homo sapiens
QVQLVESGGGVVQPGRSLRLSCAASGFTFSSYGMHWVRQAPGKGLEWVAVIWYDGSNKYYVDSVKGRFTISRDNSKNTLYLQMNSLRAEDTAVYYCARDGWQQLAPFDYWGQGTLVTVSSA

SEQ ID NO: 143 - protein, 113 aa, Homo sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPRTFGQGTKVEIKR

SEQ ID NO: 144 - protein, 113 aa, Homo sapiens
DIVMTQTPLSSPVTLGQPASISCRSSQSLVHSDGNTYLSWLHQRPGQPPRLLIYKISNRFSGVPDRFSGSGAGTAFTLKISRVEAEDVGVYYCMQATQLPRTFGQGTKVEIKR